(19) United States
(12) Patent Application Publication
(10) Pub. No.: US 2026/0176338 A1
(43) Pub. Date: Jun. 25, 2026

(54) PHARMACEUTICAL COMPOSITIONS FOR TREATING OR PREVENTING TRANSTHYRETIN-MEDIATED AMYLOIDOSIS

(71) Applicant: Neurimmune AG, Schlieren (CH)

(72) Inventors: Aubin MICHALON, Baden (CH); Michael SALZMANN, Zürich (CH)

(21) Appl. No.: 19/129,825

(22) PCT Filed: Nov. 15, 2023
(86) PCT No.: PCT/EP2023/081874

§ 371 Date: May 14, 2025

Related U.S. Application Data
(60) Provisional application No. 63/383,803, filed on Nov. 15, 2022.
(60) Provisional application No. 63/503,286, filed on May 19, 2023.

(30) Foreign Application Priority Data
Nov. 15, 2022 (EP) 22207645.7

(51) Int. Cl. C07K 16/18; A61K 39/395; A61K 47/22; A61K 47/26

(52) U.S. Cl. CPC C07K 16/18; A61K 39/39591; A61K 47/22; A61K 47/26; C07K 2317/92; C07K 2317/94

(57) ABSTRACT

Transthyretin (TTR) is a soluble protein involved in thyroxin and retinol transport in the body. Under specific conditions the TTR protein adopts misfolded, misassembled and/or aggregated TTR conformations and becomes toxic, which can lead to transthyretin-mediated amyloidosis (ATTR). Provided herein are, inter alia, compositions (e.g., pharmaceutical compositions) and related articles of manufacture that contain an anti-TTR antibody or antigen fragment thereof. Additionally, provided herein are, inter alia, methods of treating or preventing ATTR using the pharmaceutical compositions described herein.

This application claims the benefit of priory to European application no. EP 22 207 645.7 and U.S. provisional application No. 63/383,803, both filed on Nov. 15, 2022, and of U.S. provisional application No. 63/503,286, filed on May 19, 2023.

The entire contents of the above-referenced patent applications are incorporated herein by reference.

### TECHNICAL FIELD

This disclosure relates to pharmaceutical compositions for treating or preventing transthyretin-mediated amyloidosis (ATTR).

### BACKGROUND

Transthyretin (TTR) is a soluble protein involved in thyroxin and retinol transport in the body.

TTR is secreted in the blood by the liver and in the cerebrospinal fluid by the choroid plexus and is also expressed in specific tissues like the pancreatic alpha cells or retinal epithelium.

Under specific conditions which have been poorly elucidated and may include acidic pH, oxidative stress and local factors, the TTR protein adopts misfolded, misassembled and/or aggregated TTR conformations and becomes toxic, which can lead to transthyretin-mediated amyloidosis (ATTR).

There exists a need for pharmaceutical formulations for preparing and administering antibodies (e.g., human anti-TTR antibodies) that target misfolded, misassembled and/or aggregated TTR.

### SUMMARY OF THE INVENTION

Provided herein are, inter alia, compositions (e.g., pharmaceutical compositions) and related articles of manufacture that contain an anti-transthyretin (TTR) antibody or antigen fragment thereof as a drug. Additionally, provided herein are, inter alia, methods of treating or preventing transthyretin-mediated amyloidosis (ATTR) in a subject in need thereof using the pharmaceutical compositions described herein.

In accordance with the present invention, pharmaceutical formulations, essentially characterized by a pH 5.3 to 6.3, preferably pH 5.8±0.1, typically comprising a histidine buffer and as excipients sucrose, polysorbate, preferably polysorbate 80, and water for infusion/injection have been developed for a recombinant human monoclonal antibody specific for the disease associated amyloidogenic form of TTR, featuring ALXN2220, also known as NI006, at a concentration of about 25 mg/ml to about 150 mg/ml, typically at about 50 mg/ml. The formulations of the present invention allow for long-term storage of the antibody in a liquid without the need of lyophilization, and its intravenous administration for a subject in need thereof. Indeed, antibody formulations of the present invention have been successfully used in a First-in-Human Study of NI006 in Patients With Amyloid Transthyretin Cardiomyopathy (ATTR-CM), wherein the formulations has been applied from 0.3 to 60 mg per kilogram of body weight without any drug-related serious adverse events and no adverse events due to the formulations; see ClinicalTrials.gov ID NCT04360434 and Garcia-Pavia et al., Phase 1 Trial of Antibody NI006 for Depletion of Cardiac Transthyretin Amyloid. N. Engl. J. Med. 389 (2023), 239-250, each of which is incorporated herein by reference.

International application WO 2019/108689 A1 discloses the preparation of lyophilized formulations of two mouse monoclonal anti-TTR antibodies, designated 9D5 and 14G8 and humanized versions thereof, wherein the most preferred formulation (Formulation F25) comprises about 50 mg/ml humanized antibody 14G8, 20 mM histidine, 240 mM sucrose and 0.04% poloxamer 188 (PX188), at a pH of 6.0; see WO 2019/108689 A1 at page 57, paragraph [00269]. Notably, WO 2019/108689 A1 also describes the investigation of other buffers and excipients, including polysorbate 20 (PS20) and polysorbate (PS80), and specifically excludes, for example formulations comprising sucrose and PS80 in combination. In addition, WO 2019/108689 A1 provides only two short-term experiments of the tested formulations in a liquid state (one week at 50° C. and two weeks at 25° C.), while the experiments for “long-term storage” (one and three months) at different temperatures have been performed with the lyophilized formulation.

In contrast, the present invention generally pertains to a liquid formulation of anti-TTR antibodies with a long shelf life, which has the advantage of ready to use without the need for reconstitution, reduced risk of errors since no steps for reconstitution are required, and faster administration, which is especially beneficial in acute settings.

The solution of this goal as characterized in the claims is illustrated in the appended Examples by way of a preferred embodiment, wherein the formulation consists essentially of anti-TTR antibody ALXN2220, also known and referred to herein as NI006, a histidine buffer, and sucrose and polysorbate 80 (PS80) as excipients with a pH at about 5.8 and a concentration of the antibody of about 50 mg/ml or about 100 mg/ml.

In particular, a formulation of the antibody at 50 mg/ml in 20 mM histidine, 6.5% or 8% (w/v) sucrose, and 0.03% (w/v) PS80 at pH 5.8, was identified in the Examples as the lead candidate for further development and clinical use, and thus represents the most preferred embodiment of the present invention as well as equivalent formulations maintaining the physicochemical properties that are critical for the stability and function of the antibody as tested and validated in the appended Examples. To make changes in the concentration of components and/or substitute excipients in the preferred formulation, one should consider the following factors to ensure the stability and efficacy of the antibody:

Histidine: acts as a buffer agent. The buffer capacity is related to the ability of histidine to maintain the pH near its pKa value. Since the pKa of histidine is approximately 6.0, at pH 5.8, it is slightly less efficient but still functional as a buffer. The buffer capacity is also influenced by the concentration of the buffering agent and the proximity of the pH to the pKa.

Sucrose: stabilizes proteins during freeze/thaw cycles and adds osmolality. Sucrose contributes significantly to the osmolality of the solution, which is important for maintaining the structural IgG antibody.

The protein of interest at a high concentration: antibodies, like all proteins, have buffering capacity due to the presence of ionizable groups in their amino acid side chains. These groups include the carboxyl groups of aspartic acid and glutamic acid, the amino groups of lysine and arginine, the imidazole group of histidine, the hydroxyl group of tyrosine, the thiol group of cysteine, and the terminal amino and carboxyl groups.

pH 5.8: The pH is slightly below the pKa of histidine, which will still provide a buffering effect. For example, if the original buffer system has a concentration of 20 mM histidine and the pH is 5.8, one could calculate the ratio of the conjugate base to the acid using the Henderson-Hasselbalch equation. To maintain the pH, one could double both the concentration of the protonated form (HA) and the deprotonated form (A-) to 40 mM. Since changing the total concentration of the buffer will affect the ionic strength and osmolality of the solution, which can have effects on the stability and solubility of proteins, the concentration and/or nature of the osmolality agent, here sucrose, may be adjusted, and the corresponding formulation tested in accordance with the Examples.

The parent antibody of NI006 has been first described in WO 2015/092077 A1 (designated as antibody NI-301.37F1) and in Michalon et al., Nat. Commun. 12 (2021), 3142 (designated as antibody NI301A). As disclosed in WO 2015/092077 A1, NI006 (NI-301.37F1) is inter alia characterized by binding to aggregated human wild-type transthyretin (wtATTR), which is shown in FIGS. 2 to 4 and 7 and described in Examples 3 to 6, and further described at page 46, last paragraph. In addition, WO 2015/092077 A1 discloses that NI006 (NI-301.37F1) does not bind to monomers and dimers of the human native transthyretin (TTR) as shown in Example 5 and FIG. 4. This binding profile is advantageous since the antibody binds selectively to aggregated wtTTR and thus allows prima facie to consider not only the treatment of hereditary transthyretin amyloidosis (hATTR) with polyneuropathy (formerly known as Familial Amyloid Polyneuropathy, FAP), which is due to mutations in the gene encoding TTR, but also the treatment of wild-type transthyretin amyloidosis (wtATTR), known as senile systemic amyloidosis (SSA). Moreover, the antibody is not at risk of interfering with native monomer assembly into physiological tetramers. Thus, in a preferred embodiment of the pharmaceutical antibody formulation of the present invention, the antibody is ALXN2220/NI006 or an equivalent antibody that substantially has the TTR binding profile of NI006 and preferably is of human origin. For example, WO 2015/092077 A1 discloses two further human antibodies which show the mentioned binding profile, i.e., antibodies NI-301.59F1 and NI-301.35G11, and two human antibodies, NI-301.28B3 and NI301.12D3 which have the substantially same epitope as NI006 (NI-301.37F7).

Though human derived antibodies are particularly preferred, for example because by nature they are less prone to an anti-drug antibody (ADA) response, humanized antibodies and human sequence monoclonal antibodies from animals such as mice are included in the formulation of the present invention. In addition, as mentioned before. the subject formulation has been proven safe and effective; see ClinicalTrials.gov ID NCT04360434 and Garcia-Pavia et al., (2023), supra.

As illustrated in the appended Examples, several stability tests that have been performed with the formulation and pharmaceutical compositions of the present invention demonstrate that the antibody remains stable under various conditions, e.g., at 40±2° C. and 75±5% relative humidity (RH) for at least 1 month (stress stability studies); at 25±2° C./60±5% RH for at least 6 months (accelerated stability studies); and at 5±3° C. for at least 12 months to 18 months (long-term stability studies).

Accordingly, the present invention relates to a pharmaceutical composition (also referred to as pharmaceutical formulation herein) including a human anti-transthyretin (TTR) antibody or antigen-binding fragment thereof capable of binding mutated, misfolded, misassembled and/or aggregated TTR species, in particular aggregated human TTR species, and/or fragments thereof, and does not substantially recognize physiological TTR species, in which the pharmaceutical composition includes one or more of sucrose, polysorbate, preferably polysorbate 80, and a polar excipient (e.g., histidine, such as L-histidine and/or L-histidine monohydrochloride or a pharmaceutically acceptable salt thereof), preferably wherein the antibody remains stable at 40±2° C. and 75±5% RH for at least 1 week, preferably for at least up to 1 month; at 25±2° C./60±5% RH for at least 1 month, and preferably for at least up to 6 months; and/or at 5±3° C. for at least 1 month, and preferably for at least up to 12 months, more preferably for at least up to 18 months. In a preferred embodiment, the pharmaceutical composition is an aqueous composition, also referred to as liquid formulation and thus, also comprises water for injection.

The polar excipient included in the pharmaceutical composition of the present invention is also referred to as buffer system, preferably a histidine buffer (e.g., composed of L-histidine and L-histidine monohydrochloride or pharmaceutically acceptable salts thereof).

In a preferred embodiment, the polar excipient (e.g., histidine, such as L-histidine and/or L-histidine monohydrochloride or a pharmaceutically acceptable salt thereof) is present in the pharmaceutical composition of the present invention in an amount of from about 1 mM to about 100 mM (e.g., from about 1 mM to about 50 mM, e.g., about 1 mM, about 5 mM, about 10 mM, about 15 mM, about 20 mM, about 25 mM, about 30 mM, about 35 mM, about 40 mM, about 45 mM, or about 50 mM, e.g., from about 10 mM to about 30 mM, e.g., about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, e.g., from about 50 mM to about 100 mM, e.g., about 50 mM, about 55 mM, about 60 mM, about 65 mM, about 70 mM, about 75 mM, about 80 mM, about 85 mM, about 90 mM, about 95 mM, or about 100 mM), and most preferably, the polar excipient includes about 20 mM histidine (e.g., L-histidine and/or L-histidine monohydrochloride or a pharmaceutically acceptable salt thereof, preferably provided by 1.06 mg/mL L-histidine and 2.78 mg/mL L-histidine monochloride).

In addition, or alternatively, the pharmaceutical composition of the present invention includes from about 6% to about 9% (e.g., about 6%, 6.5%, 7%, 7.5%, 8%, 8.5%, or 9%) weight per unit volume (w/v) sucrose.

Preferably, the pharmaceutical composition of the present invention includes from about 6% to about 7% (e.g., about 6%, about 6.1% about 6.2%, about 6.3%, about 6.4%, about 6.5% about 6.6%, about 6.7%, about 6.8%, about 6.9%, or about 7%) w/v sucrose, and more preferably, the pharmaceutical composition of the present invention includes about 6.5% w/v sucrose.

Alternatively, the pharmaceutical composition of the present invention includes from about 7.5% to about 8.5% (e.g., about 7.5%, about 7.6%, about 7.7%, about 7.8%, about 7.9%, about 8%, about 8.1%, about 8.2%, about 8.3%, about 8.4%, or about 8.5%) w/v sucrose, and most preferably, the pharmaceutical composition of the present invention includes about 8% w/v sucrose.

Furthermore, in any of the preceding embodiments, the pharmaceutical composition of the present invention includes from about 0.001% to about 0.1% w/v (e.g., from about 0.001% to about 0.01%, e.g., about 0.001%, 0.002%, 0.003%, 0.004%, 0.005%, 0.006%, 0.007%, 0.008%, 0.009%, or 0.01%, e.g., from about 0.01% to about 0.1%, e.g., about 0.02%, 0.03%, 0.04%, 0.05%, 0.06%, 0.07%, 0.08%, 0.09%, or 0.1%) polysorbate, preferably polysorbate 80.

More preferably, the pharmaceutical composition of the present invention includes from about 0.01% to about 0.05% w/v (e.g., about 0.01%, about 0.02%, about 0.03%, about 0.04%, or about 0.05%) polysorbate 80, and most preferably, the pharmaceutical composition of the present invention includes about 0.03% w/v polysorbate 80.

The pharmaceutical composition of the present invention has in a preferred embodiment a pH of about 5.3 to about 6.3 (e.g., from about 5.3 to about 5.8, e.g., about 5.3, 5.4, 5.5, 5.6, 5.7, or 5.8, e.g., from about 5.5 to about 6.0, e.g., about 5.5, 5.6, 5.7, 5.8, 5.9, or 6.0, e.g., from about 5.8 to about 6.3, e.g., about 5.8, 5.9, 6.0, 6.1, 6.2, or 6.3).

Most preferably, the pharmaceutical composition of the present invention has a pH of about 5.8.

In principle, the antibody contained in the pharmaceutical composition can be any anti-TTR antibody, which recognizes the amyloidogenic form of TTR, i.e., aggregated TTR species, and preferably human aggregated TTR, but does not bind to physiological TTR species. In a particular preferred embodiment, the anti-TTR antibody to be used in the pharmaceutical composition of the present invention is NI006/ALXN2220 or an equivalent antibody derived from human antibody NI-301.37F1 as characterized in WO 2015/092077 A1 and in Michalon et al., Nat Commun. 12 (2021), 3142; see also supra.

Accordingly, in preferred embodiments, the anti-TTR antibody or antigen-binding fragment thereof contained in the pharmaceutical composition of the present invention includes a heavy chain variable region (VH) having the three complementary determining regions (CDRs) set forth in SEQ ID NOs: 1-3 and a light chain variable region (VL) having the three CDRs set forth in SEQ ID NOs: 4-6.

In some embodiments, the VH region includes an amino acid sequence having at least 80% sequence identity (e.g., at least 85%, 90%, 95%, 97%, 99%, or 100% sequence identity) to SEQ ID NO: 7 and the VL includes an amino acid sequence having at least 80% sequence identity (e.g., at least 85%, 90%, 95%, 97%, 99%, or 100% sequence identity) to SEQ ID NO: 8.

In some embodiments, the VH region includes an amino acid sequence having at least 80% sequence identity (e.g., at least 85%, 90%, 95%, 97%, 99%, or 100% sequence identity) to SEQ ID NO: 11 and the VL includes an amino acid sequence having at least 80% sequence identity (e.g., at least 85%, 90%, 95%, 97%, 99%, or 100% sequence identity) to SEQ ID NO: 12.

Preferably, the VH region includes the amino acid sequence of SEQ ID NO: 7 and the VL includes the amino acid of SEQ ID NO: 8, or the VH region includes the amino acid sequence of SEQ ID NO: 11 and the VL includes the amino acid of SEQ ID NO: 12.

In some embodiments, the VH region of the anti-TTR antibody or antigen binding fragment thereof includes one or more CDR sequences including an amino acid sequence having about 80%, about 85%, about 86%, about 87%, about 88%, about 89%, about 90%, about 91%, about 92%, about 93%, about 94%, about 95%, about 96%, about 97%, about 98%, about 99% or about 100% sequence identity to the amino acid sequence of SEQ ID NO: 1, SEQ ID NO: 2, and/or SEQ ID NO: 3, and the VL region of the anti-TTR antibody or antigen binding fragment thereof includes one or more CDR sequences including an amino acid sequence having about 85%, about 86%, about 87%, about 88%, about 89%, about 90%, about 91%, about 92%, about 93%, about 94%, about 95%, about 96%, about 97%, about 98%, about 99% or about 100% sequence identity to the amino acid sequence of SEQ ID NO: 4, SEQ ID NO: 5, and/or SEQ ID NO: 6. In some aspects, the VH region of the anti-TTR antibody or antigen binding fragment thereof includes one or more CDR sequences having an amino acid sequence with 1, 2, or 3 mismatches relative to the amino acid sequence of SEQ ID NO: 1, SEQ ID NO: 2, and/or SEQ ID NO: 3, and the VL region of the anti-TTR antibody or antigen binding fragment thereof includes one or more CDR sequences having an amino acid sequence with 1, 2, or 3 mismatches relative to the amino acid sequence of SEQ ID NO: 4, SEQ ID NO: 5, and/or SEQ ID NO: 6.

To avoid generation of “anti-drug antibodies” (ADA) by a subject administered an antibody or antigen-binding fragment thereof described herein, the antibody is preferably a human or humanized antibody, typically human IgG and most preferably a human IgG1. In a preferred embodiment, the antibody is a human IgG1m3 allotype. Hence, preferably the anti-TTR antibody to be used in the pharmaceutical composition of the present invention is NI006/ALXN2220 or an equivalent antibody derived from human antibody NI-301.37F1 as characterized in WO 2015/092077 A1 and in Michalon et al., Nat Commun. 12 (2021), 3142. Antibody NI006/ALXN2220 is a fully human IgG1m3 allotype antibody and thus comprises the human constant heavy chain (HC) amino acid sequence present in SEQ ID NO: 9 and a corresponding human constant light chain (LC), here kappa light chain as exemplified in SEQ ID NO: 10. As explained further below, IgG antibodies are made up as tetramers consisting of HC and two light LC chains linked by disulfide bridges.

Antibody NI006/ALXN2220 can be produced in Chinese hamster ovary (CHO) cells. CHO cells are the most widely used mammalian cells for the production of recombinant monoclonal antibodies due to their ability to perform post-translational modifications (PTMs) on the antibody molecules, which typically take place in the human body as well. Through genetic manipulation by mutagenesis, different CHO daughter cells with improved qualities have been established. Among those variants are CHO-K1, CHO-S, CHO-DXB11 and CHO-DG44. Thus, in one embodiment, the antibody used in the pharmaceutical formulation of the present invention is produced in CHO cells, preferably in CHO-K1 cells and is purified from the cell culture medium for further use.

As shown in Example 3, the major PTMs that have been identified in antibody NI006/ALXN2220 are the modification in the HC of glutamine at the N-terminus to pyro-glutamic acid, the loss of C-terminal lysine, and N-glycosylation. In this context, the N-glycosylation site was identified at position Asn300 (HC N300, SEQ ID NO: 9).

Thus, in one embodiment, the heavy chain of the anti-TTR antibody present in the pharmaceutical composition of the present invention has lost the C-terminal lysine, i.e., the antibody has undergone C-terminal lysine clipping. In particular, the C-terminal lysine as shown in SEQ ID NO: 9 is chopped off the heavy chain of the antibody, preferably from each heavy chain of the antibody. Said sequence, i.e., the sequence of the heavy chain with a clipped off C-terminal lysine is set forth in SEQ ID NO: 13. Alternatively, the glutamine at the N-terminal is modified as pyro-glutamic acid, i.e., the heavy chain of the antibody as shown in SEQ ID NO: 9 has undergone N-terminal glutaminyl cyclization. Said sequence, i.e., the sequence of the heavy chain which comprises cyclic pyroglutamic acid and no N-terminal glutamine is set forth in SEQ ID NO: 14.

Alternatively, the heavy chain of the anti-TTR antibody present in the pharmaceutical composition of the present invention has lost the C-terminal lysine and the glutamine at the N-terminal is modified as pyro-glutamic acid. Said sequence, i.e., the sequence of the heavy chain with a clipped off C-terminal lysine and which comprises cyclic pyroglutamic acid and no N-terminal glutamine is set forth in SEQ ID NO: 15.

In addition, or alternatively, the antibody is glycosylated, in particular N-glycosylated. More particularly, the heavy chain of the antibody is glycosylated and even more particularly at N300 of the heavy chain.

In a preferred embodiment, the anti-TTR antibody present in the pharmaceutical composition of the present invention lacks the C-terminal lysine, has a modified glutamine at the N-terminus as pyro-glutamic acid and comprises at least one N-glycosylation site. Thus, in one preferred embodiment, the pharmaceutical formulation of the present invention comprises an anti-TTR antibody as defined hereinbefore, wherein the antibody is composed of two heavy chains having SEQ ID NO: 9, and two light chains having SEQ ID: 10, and wherein in the heavy chain the glutamine at the N-terminus is modified as pyro-glutamic acid, the C-terminal lysine is lost, and the heavy chain is N-glycosylated. In other words, the pharmaceutical formulation of the present invention comprises in a preferred embodiment an anti-TTR antibody as defined hereinbefore, wherein the antibody is composed of two heavy chains having SEQ ID NO: 15, and two light chains having SEQ ID NO: 10, and wherein the heavy chain is N-glycosylated.

As shown in Example 3, about 99% to 100% of the antibodies present in a sample of a typical antibody formulation have a N-terminal pyro-glutamic acid in the heavy chain and about 96% of the antibodies have a loss of the C-terminal lysine. Thus, in one embodiment, about 99% of the antibodies in the formulation of the present invention have a heavy chain wherein the N-terminal pyro-glutamic acid is modified from N-terminal glutamine and/or about 96% of the antibodies have a loss of the C-terminal lysine.

In some embodiments, the anti-TTR antibody or antigen-binding fragment thereof is present in the pharmaceutical composition of the present invention at a concentration of about 1 mg/mL to about 500 mg/mL (e.g., from about 1 mg/mL to about 100 mg/mL, e.g., about 1 mg/mL, about 5 mg/mL, about 10 mg/mL, about 25 mg/mL, about 50 mg/mL, about 75 mg/mL, or about 100 mg/mL, e.g., from about 100 mg/mL to about 200 mg/mL, e.g., about 100 mg/mL, about 110 mg/mL, about 125 mg/mL, about 150 mg/mL, about 175 mg/mL, or about 200 mg/mL, e.g., from about 200 mg/mL to about 300 mg/mL, e.g., about 200 mg/mL, about 210 mg/mL, about 225 mg/mL, about 250 mg/mL, about 275 mg/mL, or about 300 mg/mL, e.g., from about 300 mg/mL to about 400 mg/mL, e.g., about 300 mg/mL, about 310 mg/mL, about 325 mg/mL, about 350 mg/mL, about 375 mg/mL, or about 400 mg/mL, e.g., from about 400 mg/mL to about 500 mg/mL, e.g., about 400 mg/mL, about 410 mg/mL, about 425 mg/mL, about 450 mg/mL, about 475 mg/mL, or about 500 mg/mL).

In a preferred embodiment, the anti-TTR antibody or antigen-binding fragment thereof is present in the pharmaceutical composition at a concentration of about 1 mg/mL to about 150 mg/mL (e.g., about 1 mg/mL to about 20 mg/mL, e.g., about 1 mg/mL, about 5 mg/mL, about 10 mg/mL, about 15 mg/mL, or about 20 mg/mL, e.g., about 20 mg/mL to about 40 mg/mL, e.g., about 20 mg/mL, about 25 mg/mL, about 30 mg/mL, about 35 mg/mL, or about 40 mg/mL, about 40 mg/mL to about 60 mg/mL, e.g., about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, or about 60 mg/mL, e.g., about 60 mg/mL to about 80 mg/mL, e.g., about 60 mg/mL, about 65 mg/mL, about 70 mg/mL, about 75 mg/mL, or about 80 mg/mL, e.g., about 80 mg/mL to about 100 mg/mL, e.g., about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, or about 150 mg/mL). In some embodiments, the anti-TTR antibody or antigen-binding fragment thereof is present in the pharmaceutical composition at a concentration of about 1 mg/mL to about 150 mg/m and in a volume of about 0.1 mL to about 100 mL (e.g., the anti-TTR antibody or antigen-binding fragment thereof is present in the pharmaceutical composition at a concentration of about 50 mg/mL, about 100 mg/mL, about 125 mg/mL, or about 150 mg/mL and in a volume of about 2 mL or 20 mL)

Even more preferably, the anti-TTR antibody or antigen-binding fragment thereof is present in the pharmaceutical composition at a concentration of about 50 mg/mL, about 100 mg/mL, about 125 mg/mL, or about 150 mg/mL, most preferably of about 50 mg/mL.

As mentioned above, the anti-TTR antibody or antigen-binding fragment thereof comprising in the pharmaceutical composition of the present invention is preferably a human IgG1 antibody. For example, the human IgG1 antibody has a heavy chain variable region with the amino acid sequence of SEQ ID NO: 7 or 11 and a light chain variable region with the amino acid of SEQ ID NO: 8. In some embodiments, the anti-TTR antibody or antigen-binding fragment thereof does not elicit an anti-drug antibody (ADA) response.

In some embodiments, the pharmaceutical composition of the present invention is present in a volume of about 0.1 mL to about 100 mL (e.g., about 0.1 mL to about 20 mL, e.g., about 1 mL, about 2 mL, about 2.25 mL, about 3 mL, about 5 ml, about 7 mL, about 10 mL, about 12 mL, about 15 mL, about 17 mL, or about 20 mL, e.g., about 20 mL to about 40 mL, e.g., about 20 mL, about 22.5 mL, about 25 mL, about 30 mL, about 35 mL, or about 40 mL, e.g., about 40 mL to about 60 mL, e.g., about 40 mL, about 45 mL, about 50 mL, about 55 mL, or about 60 mL, e.g., about 60 mL to about 80 mL, e.g., about 60 mL, about 65 mL, about 70 mL, about 75 mL, or about 80 mL, e.g., about 80 mL to about 100 mL, e.g., about 80 mL, about 85 mL, about 90 mL, about 95 mL, or about 100 mL).

Preferably, the pharmaceutical composition of the present invention is present in a volume of about 1 mL to about 10 mL (e.g., about 1 mL to about 2 mL, e.g., about 1.1 mL, about 1.2 mL, about 1.3 mL, about 1.4 mL, about 1.5 mL, about 1.6 mL, about 1.7 mL, about 1.8 mL, about 1.9 mL, or about 2 mL, e.g., about 1.5 mL to about 2.5 mL, e.g., about 1.5 mL, about 1.6 mL, about 1.7 mL, about 1.8 mL, about 1.9 mL, about 2 mL, about 2.1 mL, about 2.2 mL, about 2.3 mL, about 2.4 mL, or about 2.5 mL, e.g., about 2 to about 2.25 mL, e.g., about 2.1 mL, about 2.15 mL, about 2.2 mL, or about 2.25 mL, e.g., about 2 mL to about 5 mL, e.g., about 2 mL, about 2.5 mL, about 3 mL, about 3.5 mL, about 4 mL, about 4.5 mL, or about 5 mL, e.g., about 5 mL to about 10 mL, e.g., about 5 mL, about 5.5 mL, about 6 mL, about 6.5 mL, about 7 mL, about 7.5 mL, about 8 mL, about 8.5 mL, about 9 mL, about 9.5 mL, or about 10 mL).

More preferably, the pharmaceutical composition of the present invention is present in a volume of about 2 mL to about 20 mL (e.g., about 2 mL, about 2.25 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, or about 20 mL).

Alternatively, the pharmaceutical composition of the present invention is present in a volume of about 20 mL to about 25 mL (e.g., about 20 mL, about 20.5 mL, about 22 mL, about 22.5 mL, about 23 mL, about 23.5 mL, about 24 mL, about 24.5 mL, or about 25 mL).

Most preferably, the pharmaceutical composition of the present invention is present in a volume of about 2 mL or about 20 mL.

Furthermore, the compatibility of the antibody formulation with clinical in-use materials was evaluated. In particular, the compatibility with polyvinyl chloride IV bag, IV line, and filter as well as with polyvinyl chloride syringe and corresponding non-polyvinyl chloride materials were assessed when using glucose or saline as diluent. Three concentrations 1 mg/mL, 20 mg/mL, and 50 mg/mL were assessed, and the data provided in Tables 52 and 53 in Example 2 indicate that the formulation is in general compatible with the clinical in-use materials evaluated. In particular, in the saline group, visible particles were observed, indicating that NI006/ALXN2220 is less stable in saline, wherein the data show that when diluted in a glucose solution, no substantial changes in the appearance, protein concentration, sub-visible particle, SEC-HPLC, and ELISA binding assay were observed. Thus, NI006/ALXN2220 at concentrations of 1.0 mg/mL, 20.0 mg/mL, and 50.0 mg/mL was shown to be stable at 2-8° C. for 24 hours followed by 25° C. for 6 hours (30 hours in total) and being compatible with the clinical in-use materials evaluated.

The most common route for the administration of monoclonal antibodies in therapy is intravenous (IV) infusion. This method is preferred because it allows the antibody to be delivered directly into the bloodstream, ensuring immediate distribution throughout the body and enabling precise dosing.

Intravenous administration is particularly important for monoclonal antibodies due to their large molecular size, which generally prevents them from being effectively absorbed through the gut or skin. This means that oral or transdermal delivery methods are not suitable for these types of drugs. Additionally, IV administration bypasses the first-pass metabolism in the liver, which can significantly alter the drug's efficacy and safety profile.

Therefore, the formulation of the present invention has been particularly developed for IV administration. Thus, in a preferred embodiment, the formulation of the present invention is suitable for intravenous administration.

In some embodiments, the pharmaceutical composition has not been reconstituted from a lyophilized anti-TTR antibody or antigen-binding fragment thereof and/or is not further lyophilized.

Since in the formulation of the present invention sucrose is used as tonicity modifier and additional stabilizer of the antibody, there is no need for NaCl, especially if the primary purpose of NaCl is to stabilize the protein, which sucrose can accomplish without increasing the ionic strength of the solution, Therefore, the pharmaceutical composition of the present invention is preferably essentially free of sodium chloride.

In addition, or alternatively, the pharmaceutical composition of the present invention is essentially free of (or, e.g., completely lacks) a poloxamer.

In some embodiments, the pharmaceutical composition of the present invention is characterized by one, two, three or all four stability criteria (a) to (d) in any combination: (a) the main peak decline under heat stress conditions for 4 weeks at about 40° C. and/or 12 weeks at about 25° C. and is less than 1% of antibody by weight as measured by Size Exclusion Chromatography (SEC)-HPLC analysis; (b) the pharmaceutical composition shows a content of acidic species of the anti-TTR antibody under heat stress conditions for 2 weeks at about 40° C. of less than 42.5% as measured by Capillary Isoelectric Focusing (cIEF); (c) the pharmaceutical composition shows no substantial change in the content of acidic species of the anti-TTR antibody after 3 cycles freeze thaw (about −70° C. to RT) as measured by Capillary Isoelectric Focusing (cIEF); and/or (d) the anti-TTR antibody retains binding potency to a TTR protein of at least 80% after storage for 4 weeks at about 40° C. and/or of at least 70% to a TTR protein after storage for 12 weeks at about 25° C., e.g., as measured by ELISA and relative to a control (e.g., without prolonged storage).

In some embodiments, the pharmaceutical composition shows a main peak≥50.0%, an acidic peak≤40.0%, and a basic peak≤15.0% as measured, e.g., by cIEF, a main peak (monomer)≥95.0% and High Molecular Weight Species (HMWS)≤5.0% as measured by, e.g., Size Exclusion Chromatography (SEC)-HPLC analysis, a pH 5.8±0.5, an osmolality 240 mOsm/Kg, and an antibody concentration of 50±5.0 mg/mL.

In addition, the pharmaceutical composition of the present invention may be characterized by one, two, three or all four stability criteria (i) to (iv) in any combination: (i) the main peak decline (representing the monomer content) under heat stress conditions for 1 month at about 40° C., or for 6 months at about 25° C., or during long-term storage for 18 months at about 5° C. is less than 5%, preferably less than 4%, more preferably less than 3%, more preferably less than 2% as measured by SEC-HPLC; (ii) the pharmaceutical composition shows a content of acidic species of the anti-TTR antibody under heat stress conditions for 2 weeks at about 40° C., or for 3 months at about 25° C. of less than or about equal to about 40% as measured by cIEF; (iii) the pharmaceutical composition shows a content of acidic species of the anti-TTR antibody under long-term storage conditions for 12 months or 18 months at about 5° C. of less than 40%, preferably of less than 35% as measured by cIEF; and/or (iv) the anti-TTR antibody retains binding potency to a TTR protein of at least 80%, preferably of at least 90% after storage for 6 months at about 25° C., or after storage for 12 months or 18 months at about 5° C. as measured by ELISA and relative to a control (e.g., without prolonged storage). Accordingly, the pharmaceutical composition can be referred to as stable formulation. As explained in Example 2, the shelf life for the drug product is currently set at 24 months stored at 5±3° C., protected from light. Thus, in one embodiment, the pharmaceutical composition, i.e., the formulation has a shelf-life of 24 months at 2-8° C. when protected from light.

In addition, or alternatively, the pharmaceutical composition of the present invention has an osmolality of ≥240 mOsm/Kg and includes sucrose, and optionally a surfactant.

In some embodiments, the pharmaceutical composition of the present invention is sterile, especially for pharmaceutical use.

In some embodiments, the pharmaceutical composition of the present invention is stable upon freezing and thawing.

In some embodiments, the pharmaceutical composition of the present invention is present in a vial, such as a 2 mL or 20 mL Type I clear glass vial.

In a preferred embodiment, the vial contains an approximate 12.5% volume overfill, or a total volume of approximately 2.25 mL or 22.5 mL of the anti-TTR antibody or antigen-binding fragment thereof.

In preferred embodiment, the pharmaceutical composition of the present invention is a stable formulation and comprises an anti-TTR antibody as defined hereinbefore, preferably which comprises an immunoglobulin heavy chain (HC) comprising the amino acid sequence of SEQ ID NO: 9, 13, 14 or 15, preferably of SEQ ID NO: 9 or 15, most preferably of SEQ ID NO: 15 (i.e., SEQ ID NO: 9 with the above-indicated PTMs) and an immunoglobulin light chain (LC) comprising the amino acid sequence of SEQ ID NO: 10 at a concentration of about 25 mg/ml to about 150 mg/ml; histidine at a concentration of about 20 mM; sucrose at a concentration of about 50 mg/ml to about 80 mg/ml; polysorbate 80 (PS) at a concentration of about 0.01% (w/v) to about 0.1% (w/v); water for injection, wherein the pharmaceutical composition has a pH of about 5.3 to about 6.3.

In an overall preferred embodiment, the pharmaceutical composition of the present invention includes 8% w/v sucrose, 0.03% w/v polysorbate 80, 20 mM histidine (e.g., L-histidine), a pH of 5.8, and a volume of about or exactly 2.0 mL; and the anti-TTR antibody or an antigen-binding fragment thereof includes a heavy chain variable (VH) region with the amino acid sequence of SEQ ID NO: 7 or 11, preferably of SEQ ID NO: 11, and light chain variable (VL) region with the amino acid sequence of SEQ ID NO: 8, preferably wherein the antibody is a fully human IgG1m3 allotype antibody and thus comprises the human heavy chain (HC) amino acid sequence present in SEQ ID NO: 9 and the human light chain (LC), here kappa light chain as exemplified in SEQ ID NO: 10, preferably with the above-indicated PTMs, i.e., preferably a HC amino acid sequence present in SEQ ID NO: 15. Preferably, the pharmaceutical composition comprises that antibody at a concentration of 50 mg/mL or 100 mg/mL, most preferably of 50 mg/mL.

Alternatively, the pharmaceutical composition includes 6.5% w/v sucrose, 0.03% w/v polysorbate 80, 20 mM histidine (e.g., L-histidine), a pH of 5.8, and a volume of about or exactly 2.0 mL; and the anti-TTR antibody or an antigen-binding fragment thereof includes a VH region with the amino acid sequence of SEQ ID NO: 7 or 11, preferably of SEQ ID NO: 11, and VL region with the amino acid sequence of SEQ ID NO: 8, preferably wherein the antibody a fully human IgG1m3 allotype antibody and thus comprises the human heavy chain (HC) amino acid sequence present in SEQ ID NO: 9 and the human light chain (LC), here kappa light chain as exemplified in SEQ ID NO: 10, preferably with the above-indicated PTMs, i.e., preferably a HC amino acid sequence present in SEQ ID NO: 15. Preferably, the pharmaceutical composition comprises that antibody at a concentration of 50 mg/mL or 100 mg/mL, most preferably of 50 mg/mL.

The present invention further relates to a kit including the pharmaceutical composition of the present invention and instructions for use thereof.

In some embodiments, the kit is for use in treating or preventing transthyretin-mediated amyloidosis (ATTR) in a human subject.

The present invention further relates to a method of treating ATTR in a human subject including administering the pharmaceutical composition of the present invention to the human subject.

The present invention further relates to a method of treating ATTR amyloidosis with cardiomyopathy (ATTR-CM) or sporadic wild-type-ATTR-CM (e.g., a wild type ATTR gene that codes for TTR proteins that form deposits in the heart) in a human subject including administering the pharmaceutical composition of the present invention to the human subject.

The present invention further relates to a pharmaceutical composition including a human anti-transthyretin (TTR) antibody or antigen-binding fragment thereof at a concentration of about 50 mg/mL to about 150 mg/mL (e.g., 50 mg/mL, about 60 mg/mL, 70 mg/mL, about 80 mg/mL, 90 mg/mL, about 100 mg/mL, about 110 mg/mL, about 120 mg/mL, about 130 mg/mL, about 140 mg/mL, or about 150 mg/mL), a histidine (e.g., L-histidine) buffer with a pH of about 5.8, 6.5%, or 8.0% w/v of sucrose, and 0.03% w/v of polysorbate 80; wherein the anti-TTR antibody or antigen-binding fragment thereof is capable of binding mutated, misfolded, misassembled and/or aggregated TTR species and/or fragments thereof and does not substantially recognize physiological TTR species.

The present invention further provides a medicament including the pharmaceutical composition of the present invention, e.g., in which the medicament is for use in a method of treating ATTR in a subject in need thereof, such as for use in a treatment of a subject with ATTR amyloidosis with cardiomyopathy (ATTR-CM), such as sporadic WT-ATTR-CM.

The present invention further relates to a pharmaceutical container, preferably a sterile container, including the pharmaceutical composition of the present invention or the medicament of the present invention, in which, e.g., the container is a single-use glass vial that includes about 100 mg of the antibody in a concentration of about 50±5 mg/mL to about 150 mg/mL (e.g., about 50 mg/mL, about 60 mg/mL, 70 mg/mL, about 80 mg/mL, 90 mg/mL, about 100 mg/mL, about 110 mg/mL, about 120 mg/mL, about 130 mg/mL, about 140 mg/mL, or about 150 mg/mL, such as about 100 mg/mL), optionally in which the vial contains an approximate 10% or 12.5% volume overfill.

In a preferred embodiment, the pharmaceutical composition of the present invention is ready-to use for administration to a subject in need thereof, such as via intravenous infusion, with or without dilution. Preferably, the pharmaceutical composition is a sterile, colorless to slightly yellow, clear to slightly opalescent liquid, essentially free of visible particles for intravenous use by infusion after dilution.

Preferably, the pharmaceutical composition is diluted prior to infusion in a solution containing a glucose or a polymer thereof (e.g., dextran). Thus, preferably, glucose or a polymer thereof (e.g., dextran) is used as diluent and prior to infusion, the pharmaceutical composition is added to the diluent. In some embodiments, the concentration of the glucose or the polymer thereof is 5% w/v. In some embodiments, the antibody in the pharmaceutical composition is not diluted below 1 mg/mL.

In some embodiments, the anti-TTR antibody or antigen binding fragment thereof is administered to a subject in an aqueous formulation at a diluted concentration of about 1 mg/mL to about 50 mg/mL (e.g., about 1 mg/mL to about 42 mg/mL, about 1 mg/mL to about 30 mg/mL, about 1 mg/mL to about 20 mg/mL, or about 1 mg/mL to about 10 mg/mL, e.g., about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL).

The present invention further relates a method of treating ATTR (e.g., ATTR-CM or WT-ATTR-CM) in a human subject by administering the pharmaceutical composition of the present invention to the subject. In an embodiment, the method includes administering a dose of the pharmaceutical composition providing about 1-60 mg/kg (e.g., about 10-60 mg/kg, about 30-60 mg/kg, about 30 mg/kg, or about 60 mg/kg) of the human anti-TTR antibody or about 3000-7500 mg (e.g., about 4000-7000 mg, about 3000-6000 mg, or about 4000-5000 mg, e.g., about 3000 mg, about 3500 mg, about 4000 mg, about 4500 mg, about 5000 mg, about 5500 mg, about 6000 mg, about 6500 mg, about 7000 mg, or about 7500 mg) of the human anti-TTR antibody to the subject. In an embodiment, about 3000 mg of the human anti-TTR antibody or antigen-binding fragment thereof is administered to the human subject. In an embodiment, about 7500 mg of the human anti-TTR antibody or antigen-binding fragment thereof is administered to the human subject.

The present invention further relates to a method of treating ATTR (e.g., ATTR-CM or WT-ATTR-CM) in a human subject by administering the pharmaceutical composition of the present invention to the subject. In an embodiment, the method includes administering a dose of the pharmaceutical composition providing about 30-60 mg/kg (e.g., about 1-60 mg/kg (e.g., about 10-60 mg/kg, about 30-60 mg/kg, about 30 mg/kg, or about 60 mg/kg) of the human anti-TTR antibody or about 3000-7500 mg (e.g., about 4000-7000 mg, about 3000-6000 mg, or about 4000-5000 mg, e.g., about 3000 mg, about 3500 mg, about 4000 mg, about 4500 mg, about 5000 mg, about 5500 mg, about 6000 mg, about 6500 mg, about 7000 mg, or about 7500 mg) of the human anti-TTR antibody to the subject. In an embodiment, about 3000 mg of the human anti-TTR antibody or antigen-binding fragment thereof is administered to the human subject. In an embodiment, about 7500 mg of the human anti-TTR antibody or antigen-binding fragment thereof is administered to the human subject.

The present invention further relates to a pharmaceutical composition of the present invention for treating ATTR (e.g., ATTR-CM or WT-ATTR-CM) in a subject in need thereof. In an embodiment, the pharmaceutical composition is for administration at a dose providing about 1-60 mg/kg (e.g., about 10-60 mg/kg, about 30-60 mg/kg, about 30 mg/kg, or about 60 mg/kg) of the human anti-TTR antibody or about 3000-7500 mg (e.g., about 4000-7000 mg, about 3000-6000 mg, or about 4000-5000 mg, e.g., about 3000 mg, about 3500 mg, about 4000 mg, about 4500 mg, about 5000 mg, about 5500 mg, about 6000 mg, about 6500 mg, about 7000 mg, or about 7500 mg) of the human anti-TTR antibody to the subject. In an embodiment, a dose of about 3000 mg of the human anti-TTR antibody or antigen-binding fragment thereof is used for treating the human subject. In an embodiment, a dose of about 7500 mg of the human anti-TTR antibody or antigen-binding fragment thereof is used for treating the human subject.

The present invention further relates to a pharmaceutical composition of the present invention for treating ATTR (e.g., ATTR-CM or WT-ATTR-CM) in accordance with the present invention in a subject in need thereof. In an embodiment, the pharmaceutical composition is for administration at a dose providing about 1-60 mg/kg (e.g., about 10-60 mg/kg, about 30-60 mg/kg, about 30 mg/kg, or about 60 mg/kg) of the human anti-TTR antibody or about 3000-7500 mg (e.g., about 4000-7000 mg, about 3000-6000 mg, or about 4000-5000 mg, e.g., about 3000 mg, about 3500 mg, about 4000 mg, about 4500 mg, about 5000 mg, about 5500 mg, about 6000 mg, about 6500 mg, about 7000 mg, or about 7500 mg) of the human anti-TTR antibody to the subject. In an embodiment, a dose of about 3000 mg of the human anti-TTR antibody or antigen-binding fragment thereof is used for treating the human subject. In an embodiment, a dose of about 7500 mg of the human anti-TTR antibody or antigen-binding fragment thereof is used for treating the human subject.

The present invention further relates to the use of a pharmaceutical composition in the manufacture of a medicament for treating or preventing ATTR in a subject. The pharmaceutical composition includes a human anti-TTR antibody or antigen-binding fragment thereof capable of binding mutated, misfolded, misassembled and/or aggregated TTR species and/or fragments thereof and does not substantially recognize physiological TTR species. The pharmaceutical composition further includes one or more (e.g., 1, 2, or 3) of sucrose, polysorbate 80, and a polar excipient. Preferably, the pharmaceutical composition is the pharmaceutical composition of the present invention.

In some embodiments, the pharmaceutical composition includes a human anti-TTR antibody or antigen-binding fragment thereof at a concentration of about 50 mg/mL to about 150 mg/mL (e.g., about 50 mg/mL, about 60 mg/mL, 70 mg/mL, about 80 mg/mL, 90 mg/mL, about 100 mg/mL, about 110 mg/mL, about 120 mg/mL, about 130 mg/mL, about 140 mg/mL, or about 150 mg/mL), a histidine buffer with a pH of about 5.8, 6.5% or 8.0% w/v of sucrose, and 0.03% w/v of polysorbate 80 in the manufacture of a medicament for treating or preventing ATTR in a subject. The anti-TTR antibody or antigen-binding fragment thereof is capable of binding mutated, misfolded, misassembled and/or aggregated TTR species and/or fragments thereof and does not substantially recognize physiological TTR species.

The present invention further relates to a method of obtaining the pharmaceutical composition of the present invention, which comprises formulating the antibody in a buffer as defined hereinbefore.

### BRIEF DESCRIPTION OF THE DRAWINGS

The accompanying drawings are included to illustrate embodiments of the disclosure and further an understanding of its implementations.

FIG. 1 is a graph depicting the size exclusion chromatography (SEC) % Main peak trend of the indicated buffers in the pH buffer study described in Example 1.

FIG. 2 is a graph depicting the capillary isoelectric focusing (cIEF) % Main peak trend of the indicated buffers in the pH screening study described in Example 1.

FIG. 3 is a graph depicting the SEC % Main peak decrease of six formulations (F) after a 40° C. incubation, as described in Example 1.

FIG. 4 is a graph depicting the SEC % Main peak decrease of the indicated buffers after a two week 40° C. incubation, as described in Example 1.

### DEFINITIONS

For the avoidance of any doubt, it is emphasized that the expressions “in some embodiments”, “in a certain embodiments”, “in certain instances”, “in some instances”, “in a further embodiment”, “in one embodiment”, “in a further aspect”, “in a first aspect”, “in a second aspect”, etc., and the like are used and meant such that any of the embodiments described therein are to be read with a mind to combine each of the features of those embodiments and that the disclosure has to be treated in the same way as if the combination of the features of those embodiments and aspects would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices. In this context, the person skilled in the art will appreciate that the embodiments and features disclosed in the Examples are intended to be generalized to any anti-TTR antibody and equivalents having substantially the same properties.

As used herein, the term “about,” as used herein, refers to a value that is ±10% of a recited value, preferably within ±5%. For example, “about 8%” can mean any percentage between 7.2% and 8.8%, preferably any percentage between 7.6% and 8.4%. In another example, “about 2 mL” can mean any volume between 1.8 mL and 1.2 mL (e.g., 1.8 mL, 1.9 mL, 1.95 mL, 2 mL, 2.05 mL, 2.10 mL, 2.15 mL, and 2.2 mL). In the context of the antibody amount as referred to herein, e.g., 50 mg/mL or 100 mg/mL, the term “about” refers to concentrations ranging from 45 mg/mL to 50 mg/mL (see the “Acceptance criterion” as defined in the Examples), and preferably from 48 mg/mL to 52 mg/mL (see for example Tables 4, 54, 56, 57, 60, 62, 64, 65, 67, and 68), and to concentrations ranging from 90 mg/mL to 113 mg/mL, preferably from 96 mg/mL to 113 mg/mL (see for example Tables 15, 20, 28, 34, and 46), respectively. Accordingly, even if the term “about” is not explicitly used, the above indicated concentration ranges apply, e.g., when reference is made to 50 mg/mL, concentrations ranging from 45 mg/mL to 55 mg/mL are included and when reference is made to 100 mg/mL, concentrations ranging from 90 mg/mL to 113 mg/mL are included since these ranges are within the experimental deviation as indicated in the above-mentioned Tables.

In connection with the present invention, the term “and/or” is understood to mean that all members of a group which are connected by the term “and/or” are disclosed cumulatively in any combination, both alternatively to each other and in each case to each other. This means for the expression “A, B and/or C” that the following disclosure content is to be understood thereunder: a) A or B or C; or b) (A and B); or c) (A and C); or d) (B and C); or e) (A and B and C).

The phrases “essentially free of NaCl” and “substantially free of NaCl” in the context of the formulation/pharmaceutical composition of the present invention mean that the pharmaceutical composition/formulation contains either no NaCl or only trace amounts that are considered negligible for the intended use of the product and, i.e., that NaCl is present at such a low level that it does not affect the formulation's performance, stability, safety, or efficacy. Furthermore, the phrases may also refer to formulations/pharmaceutical compositions to which NaCl is not intentionally added, but which may comprise either Na²⁺ or Cl⁻ due to the presence of salts of other excipients, for example histidine-HCl.

The phrases “essentially free of” and “substantially free of” in the context of the formulation/pharmaceutical composition of the present invention mean that the pharmaceutical composition/formulation does not contain the substance which is referred to or only trace amounts that are considered negligible for the intended use of the product and, i.e., that the substance is present at such a low level that it does not affect the formulation's performance, stability, safety, or efficacy.

As used herein, the term “binding potency” refers to a characteristic corresponding to a quantitative measure of biological activity (e.g., TTR-binding). A binding potency assay (e.g., an ELISA assay) can be used to measure the ability of an anti-TTR antibody or antigen-binding fragment thereof of the disclosure to elicit a specific response in a disease-relevant system (e.g., a subject having ATTR-CM, such as WT-ATTR-CM). The activity measured in the assay is a surrogate for an intended biological effect and can be used to assess the maintenance of that effect over time (e.g., following storage).

As used herein, the expressions “is capable of binding” and “binds to” refers to the capability of the antibody or antigen-binding fragment thereof to bind to, for example aggregated TTR, under experimental conditions (for example in an ELISA assay) as illustrated in the Examples. As used herein, the term “pharmaceutical composition” refers to a mixture containing a therapeutic agent (e.g., an anti-TTR antibody described herein), optionally in combination with one or more pharmaceutically acceptable excipients, diluents, and/or carriers. The pharmaceutical composition is, for example, formulated for administration to a subject, such as a mammal, e.g., a human, in order to prevent, treat or control a particular disease or condition affecting, or that may affect, the subject (e.g., ATTR-CM, such as WT-ATTR-CM).

As used herein, the term “pharmaceutically acceptable” refers to those compounds, materials, compositions, and/or dosage forms which are, within the scope of sound medical judgment, suitable for use in contact with the tissues a subject, such as a mammal (e.g., a human) without excessive toxicity, irritation, allergic response, or other problem or complication, commensurate with a reasonable benefit/risk ratio.

The term “between” as used herein includes the endpoints.

As used herein, room temperature (RT) is defined as between 15 to 25° C. according to the European Pharmacopoeia. “Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

### DETAILED DESCRIPTION

The present disclosure relates to pharmaceutical compositions containing anti-transthyretin (TTR) antibodies or antigen-binding fragments thereof that may be used in various therapeutic and prophylactic methods described herein. The pharmaceutical compositions contain antibodies or antigen-binding fragments thereof that can bind mutated, misfolded, misassembled and/or aggregated TTR species and/or fragments thereof and does not substantially recognize physiological TTR species.

The pharmaceutical compositions contain combinations of specific carriers and excipients that impart surprisingly beneficial properties to the compositions, including the ability to formulate the drug (the anti-TTR antibodies and antigen-binding fragments thereof) at a high concentration (e.g., about 150 mg/mL), to enhance stability of the composition (e.g., extended shelf-life, e.g., about 4 weeks, particularly at high temperatures (e.g., at about 40° C.), and 18 months at low temperatures (e.g., at about 5° C.), respectively), to reduce aggregation of the drug (e.g., ≤5400 particles per mL that are ≥10 μM in size), and to improve viscosity parameters of the composition (e.g., about 16 cP). The components of the pharmaceutical composition are described in more detail below.

### Antibodies and Antigen Binding Fragments Thereof

The pharmaceutical compositions described herein include an antibody or antigen-binding fragment thereof, e.g., a human anti-TTR antibody, which is capable of binding to aggregated human wild-type transthyretin (wtATTR) and preferably does not bind to monomers and dimers of the human native TTR. Preferably, the antibody is also capable of binding to mutant TTR aggregates.

Exemplary heavy chain variable (VH) region, light chain variable (VL) region, and complimentary determining regions (CDRs) of the anti-TTR antibodies described herein are shown in Table 1 below. CDR sequences were defined by the Kabat system (bioinf.org.uk/abs/). SEQ ID NO: 1 (VH-CDR1) represents residues 31-35 (Kabat numbering) of SEQ ID NO: 7 (VH). SEQ ID NO: 2 (VH-CDR2) represents residues 52-67 (Kabat numbering) of SEQ ID NO: 7 (VH). SEQ ID NO: 3 (VH-CDR3) represents residues 100-109 (Kabat numbering) of SEQ ID NO: 7 (VH).

SEQ ID NO: 4 (VL-CDR1) represents residues 31-35 (Kabat numbering) of SEQ ID NO 8 (VL). SEQ ID NO: 5 (VL-CDR2) represents residues 52-67 (Kabat numbering) of SEQ ID NO 8 (VL). SEQ ID NO: 6 (VL-CDR3) represents residues 100-109 (Kabat numbering) of SEQ ID NO: 8 (VL).

TABLE 1
Anti-TTR Antibody Sequences

| Domain/ | SEQ | |
|---|---|---|
| Region | ID NO: | Amino Acid Sequence |
| VH-CDR1 | 1 | SRSSY |
| VH-CDR2 | 2 | GIYHSGNTYDNPSLKS |
| VH-CDR3 | 3 | IVPGGDAFDI |
| VL-CDR1 | 4 | RASQSVGTYLN |
| VL-CDR2 | 5 | AASSLOS |
| VL-CDR3 | 6 | QQSYSSPPT |
| VH | 7 | QLQLQESGPGLVKPSETLSLTCSVSGGSIISRSSYWGWIRQPPGKGLEWIG |
| | | GIYHSGNTYDNPSLKSRLTMSVDTSKNQFSLNLRSVTAADTAVYYCARIVP |
| | | GGDAFDIWGQGTMVTVSS |
| VL | 8 | DIQMTQSPSSLSASVGDRVTIACRASQSVGTYLNWYQQKRGKAPKLLIFAA |
| | | SSLQSGVPSRFSGSGSGTDFTLTISSLQPEDFATYYCQQSYSSPPTFGQGT |
| | | KVEIK |
| HC | 9 | Q*LQLQESGPGLVKPSETLSLTCSVSGGSIISRSSYWGWIRQPPGKGLEWI |
| | | GGIYHSGNTYDNPSLKSRLTMSVDTSKNQFSLNLRSVTAADTAVYYCARIV |
| | | PGGDAFDIWGQGTMVTVSSASTKGPSVFPLAPSSKSTSGGTAALGCLVKD |
| | | YFPEPVTVSWNSGALTSGVHTFPAVLQSSGLYSLSSVVTVPSSSLGTQTYI |
| | | CNVNHKPSNTKVDKRVEPKSCDKTHTCPPCPAPELLGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTL |
| | | PPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK* |
| | | * the C-terminal lysine (K) is optional |
| | | and/or the N-terminal glutamine (Q) |
| | | undergoes intramolecular cyclization, |
| | | resulting in the formation of |
| | | pyroglutamate |
| LC | 10 | DIQMTQSPSSLSASVGDRVTIACRASQSVGTYLNWYQQKRGKAPKLLIFAA |
| | | SSLQSGVPSRFSGSGSGTDFTLTISSLQPEDFATYYCQQSYSSPPTFGQGT |
| | | KVEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| VH | 11 | LQLQESGPGLVKPSETLSLTCSVSGGSIISRSSYWGWIRQPPGKGLEWIGGI |
| | | YHSGNTYDNPSLKSRLTMSVDTSKNQFSLNLRSVTAADTAVYYCARIVPGG |
| | | DAFDIWGQGTMVTVSS |
| VL | 12 | DIQMTQSPSSLSASVGDRVTIACRASQSVGTYLNWYQQKRGKAPKLLIFAA |
| | | SSLQSGVPSRFSGSGSGTDFTLTISSLQPEDFATYYCQQSYSSPPTFGQGT |
| | | KVEI |
| HC - without | 13 | QLQLQESGPGLVKPSETLSLTCSVSGGSIISRSSYWGWIRQPPGKGLEWIG |
| C-terminal | | GIYHSGNTYDNPSLKSRLTMSVDTSKNQFSLNLRSVTAADTAVYYCARIVP |
| lysine | | GGDAFDIWGQGTMVTVSSASTKGPSVFPLAPSSKSTSGGTAALGCLVKDY |
| | | FPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTL |
| | | MISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTLPP |
| | | SREEMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| HC - without | 14 | LQLQESGPGLVKPSETLSLTCSVSGGSIISRSSYWGWIRQPPGKGLEWIGGI |
| N-terminal | | YHSGNTYDNPSLKSRLTMSVDTSKNQFSLNLRSVTAADTAVYYCARIVPGG |
| glutamine** | | DAFDIWGQGTMVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVN |
| | | HKPSNTKVDKRVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTLPPSR |
| | | EEMTKNQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| HC - without | 15 | LQLQESGPGLVKPSETLSLTCSVSGGSIISRSSYWGWIRQPPGKGLEWIGGI |
| C-terminal | | YHSGNTYDNPSLKSRLTMSVDTSKNQFSLNLRSVTAADTAVYYCARIVPGG |
| lysine and N- | | DAFDIWGQGTMVTVSSASTKGPSVFPLAPSSKSTSGGTAALGCLVKDYFPE |
| terminal | | PVTVSWNSGALTSGVHTFPAVLQSSGLYSLSSVVTVPSSSLGTQTYICNVN |
| glutamine** | | HKPSNTKVDKRVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTLPPSR |
| | | EEMTKNQVSLTCLVKGFYPSDIAVEWESNGQPENNYKTTPPVLDSDGSFFL |
| | | YSKLTVDKSRWQQGNVFSCSVMHEALHNHYTQKSLSLSPG |

CDR = complimentary determining region; VH = heavy chain variable region; VL = light chain variable region; HC = heavy chain; LC = light chain
**the N-terminal glutamine is absent and/or has been modified to pyroglutamate, which is also referred to as N-terminal cyclization, preferably, the N-terminal glutamine has been modified to pyroglutamate

The anti-TTR antibody or antigen binding fragment thereof may include one or more CDR sequences including an amino acid sequence having about 80%, about 85%, about 86%, about 87%, about 88%, about 89%, about 90%, about 91%, about 92%, about 93%, about 94%, about 95%, about 96%, about 97%, about 98%, about 99% or about 100% sequence identity to the amino acid sequence of SEQ ID NO: 1, SEQ ID NO: 2, SEQ ID NO: 3, SEQ ID NO: 4, SEQ ID NO: 5, and/or SEQ ID NO: 6. Further, the anti-TTR antibody or antigen binding fragment thereof may include one or more CDR sequences having an amino acid sequence with 1, 2, or 3 mismatches relative to the amino acid sequence of SEQ ID NO: 1, SEQ ID NO: 2, SEQ ID NO: 3, SEQ ID NO: 4, SEQ ID NO: 5, and/or SEQ ID NO: 6. In a particular example, the anti-TTR antibody or antigen binding fragment thereof includes six CDR amino acid sequences with 100% sequence identity to the amino acid sequence of SEQ ID NO: 1, SEQ ID NO: 2, SEQ ID NO: 3, SEQ ID NO: 4, SEQ ID NO: 5, and SEQ ID NO: 6. The anti-TTR antibody or antigen-binding fragment thereof may have a VH region including an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the amino acid sequence of SEQ ID NO: 7. In a particular example, the anti-TTR antibody or antigen-binding fragment thereof has a VH region including an amino acid sequence with 100% sequence identity to the amino acid sequence of SEQ ID NO: 7. The anti-TTR antibody or antigen-binding fragment thereof may have a VL region including an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the amino acid sequence of SEQ ID NO: 8. In a particular example, the anti-TTR antibody or antigen-binding fragment thereof has a VL region including an amino acid sequence with 100% sequence identity to the amino acid sequence of SEQ ID NO: 8.

The anti-TTR antibody or antigen-binding fragment thereof may have a VH region including an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the amino acid sequence of SEQ ID NO: 11. In a particular example, the anti-TTR antibody or antigen-binding fragment thereof has a VH region including an amino acid sequence with 100% sequence identity to the amino acid sequence of SEQ ID NO: 11.

The anti-TTR antibody or antigen-binding fragment thereof may have a VL region including an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the amino acid sequence of SEQ ID NO: 12. In a particular example, the anti-TTR antibody or antigen-binding fragment thereof has a VL region including an amino acid sequence with 100% sequence identity to the amino acid sequence of SEQ ID NO: 12.

Alternatively, the anti-TTR antibody is an anti-TTR antibody described in U.S. Pat. No. 10,344,080 or 11,180,545 (each of which is incorporated herein by reference in its entirety). In some embodiments, the anti-TTR antibody is an anti-TTR antibody described in U.S. Publication Number US 2022-0144928 (which is incorporated herein by reference in its entirety).

Also, within the scope of the disclosure are anti-TTR antibodies and antigen-binding fragments thereof, in which specific amino acids have been substituted, deleted, or added. These modifications do not have a substantial effect on the biological properties of the anti-TTR antibody, such as binding activity. For example, antibodies may have amino acid substitutions in the framework region (FR), so as to improve binding to the antigen. In another example, a number of acceptor framework residues can be replaced by the corresponding donor amino acids. The donor framework can be a mature or germline human antibody framework sequence or a consensus sequence. Guidance concerning how to make phenotypically silent amino acid substitutions is provided in, e.g., Bowie et al. (Science, 247: 1306-1310, 1990), Cunningham et al. (Science, 244: 1081-1085, 1989), Ausubel (ed.) (Current Protocols in Molecular Biology, John Wiley and Sons, Inc., 1994), T. Maniatis, E. F. Fritsch and J. Sambrook (Molecular Cloning: A Laboratory Manual, Cold Spring Harbor laboratory, Cold Spring Harbor, N.Y., 1989), Pearson (Methods Mol. Biol. 243:307-31, 1994), and Gonnet et al. (Science 256:1443-45, 1992); each of which is incorporated herein by reference.

The variant antibodies or antigen-binding fragments thereof are functionally active and may have, e.g., fewer than about 30%, about 25%, about 20%, about 15%, about 10%, about 5% or about 1% amino acid of the number of residues substituted or deleted while retaining essentially the same immunological properties including, but not limited to, binding to TTR, as described herein i.e., equivalent antibodies having substantially the same binding properties to TTR as the exemplarity antibody, which has been described in U.S. Pat. Nos. 10,344,080 B2 and 11,180,545 B2 (corresponding to international application WO 2015/092077 A1, where the antibody is named NI-301.37F1; see also supra) to comprise a heavy chain variable region with the amino acid sequence of SEQ ID NO: 7 and the light chain variable region with the amino acid of SEQ ID NO: 8. As mentioned above, this antibody is characterized in U.S. Pat. Nos. 10,344,080 B2 and 11,180,545 B2 as well as in Michalon et al., Nat. Commun. 12 (2021), 3142 (designated as antibody NI301A) by (i) binding selectively with high affinity to the disease associated ATTR aggregates, (ii) binding to misfolded/aggregated wild type TTR and variant TTR related to sporadic and hereditary disease, respectively, as well as to ATTR deposits in cardiac tissues obtained at autopsy from ATTR-CM patients, but not substantially binding native TTR monomers, and, importantly, being capable of removing ATTR fibrils by macrophage-mediated phagocytosis. The latter property can be easily tested as described in international application WO 2020/094883 A1; see also Michalon et al. (2021), supra.

The antibodies or antigen-binding fragments thereof may also include variants, including, e.g., humanized or chimeric antibodies or antigen-binding fragments thereof, analogs, orthologs, homologs and derivatives of antibodies, that exhibit a biological activity, e.g., binding of an antigen such as TTR.

The antibodies may contain one or more analogs of an amino acid (including, for example, non-naturally occurring amino acids, amino acids which only occur naturally in an unrelated biological system, modified amino acids from mammalian systems etc.), antibodies with substituted linkages, as well as other modifications known in the art.

In some embodiments, the anti-TTR antibody fragment is selected from the group consisting of bis-Fab, Fab, Fab′-SH, Fv, scFv, and (Fab′)2 fragments.

In certain embodiments, the anti-TTR antibody or antigen-binding fragment thereof is a monoclonal antibody (mAb). In some instances, the anti-TTR antibody or antigen-binding fragment thereof is an IgG antibody. In some instances, the anti-TTR antibody or antigen-binding fragment thereof is a human IgG1 antibody. In an embodiment, the human anti-TTR antibody or antigen-binding fragment thereof does not elicit an anti-drug antibody (ADA) response in a human subject. In some embodiments, the anti-TTR antibody or antigen-binding fragment thereof has a VH region including an amino acid sequence with 100% sequence identity to the amino acid sequence of SEQ ID NO: 7 and a VL region including an amino acid sequence with 100% sequence identity to the amino acid sequence of SEQ ID NO: 8.

In this context, the person skilled in the art is aware that effector function and intensity can, inter alia, depend on the IgG class or isotype and that IgG2 and IgG4 have only attenuated effector functions compared to IgG1 or IgG3. Therefore, in an embodiment, the anti-TTR antibody described herein can be of the IgG1 or IgG3 class or isotype, for example, IgG1. Of course, besides using native IgG immunoglobulins corresponding effector functions can be genetically engineered; see, e.g., Saunders KO (2019) Conceptual Approaches to Modulating Antibody Effector Functions and Circulation Half-Life. Front. Immunol. 10: 1296. Doi: 10.3389/fimmu.2019.01296.

The five primary classes of immunoglobulins are IgG, IgM, IgA, IgD and IgE. These are distinguished by the type of heavy chain found in the molecule. IgG molecules have heavy chains known as gamma-chains; IgMs have mu-chains; IgAs have alpha-chains; IgEs have epsilon-chains; and IgDs have delta-chains; see for review, e.g., Schroeder et al., J. Allergy Clin. Immunol. 125 (2010), S41-S52. Furthermore, different subclasses exist, wherein the IgAs are further divided into subclasses IgA1 and IgA2, and wherein IgGs are further divided into subclasses IgG1, IgG2, IgG3, and IgG4. Furthermore, two types of light chain, kappa (κ) and lambda (λ) exist.

In principle, the antibody as used in accordance with the present invention may be of any kind of class and subclass, respectively, and may comprise any kind of light chain, as long as the antibody binds to misfolded and preferably aggregated forms of TTR, and preferably as long as binding specificity towards TTR as indicated in the Examples of WO 2015/092077 A1 for antibody NI-301.37F1 remains unaffected in kind and as long as no adverse effects occur when administering said antibody to a subject, wherein the adverse effects can be determined as described in Example 1. However, preferably complete IgG antibodies are used, wherein the antibody comprises a constant domain. Accordingly, in one embodiment, the immunoglobulin heavy and/or light chain constant domain present in the antibody as used in accordance with the present invention is of the IgG type, the IgM type, the IgA type, the IgD type or the IgE type, preferably of the IgG type. In one embodiment, the immunoglobulin heavy and/or light chain constant domain present in the antibody as used in accordance with the present invention is of the IgA1, IgA1, IgG1, IgG2, IgG3, or IgG4 subclass, preferably of the IgG1, IgG2, IgG3, or IgG4 subclass and most preferably of the IgG1 subclass.

Recombinant expression of complete human IgG1 antibodies with a human or mouse constant domain can be performed substantially as described in the Examples of WO 2015/092077 A1. Preferably, the antibody is a monoclonal antibody or derived from a monoclonal antibody.

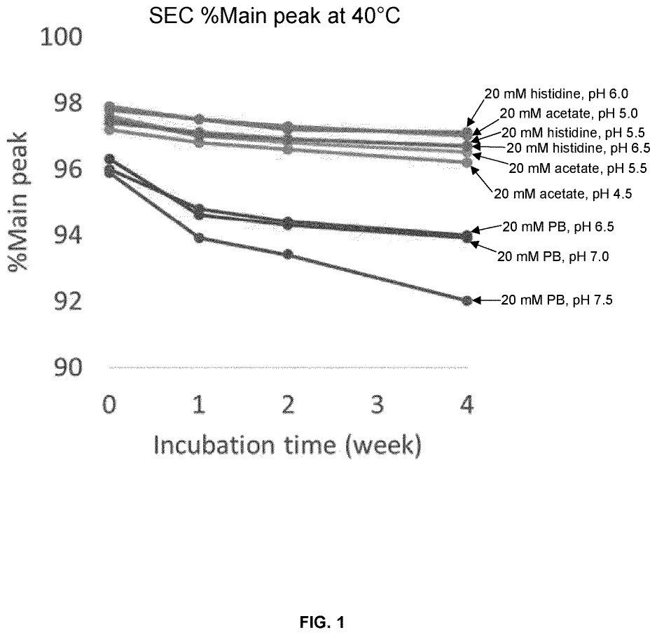

There are not only the above-mentioned four subclasses of IgGs but human heavy and light chain genes also exhibit extensive structural polymorphism(s) and, being closely linked, are inherited as a haplotype. Allotypic variants can be immunogenic and provoke antibody responses as a result of allo-immunization. Thus, switching the allotype can be of particular interest to provide non-immunogenic antibody therapeutics. So far, extensive allotypes (polymorphisms) are known, but focus is put on the serologically defined allotypes. Allotypes of IgG proteins are defined by the expression of unique epitope(s) recognized by unique serologic reagent(s). Allotypes expressed on the constant region of IgG heavy chain are designated as Gm (Genetic marker) together with the subclass, e.g., G1m, and the allotype number (or letter), e.g., G1m1 [or G1m(a)], G3m5 [or G3m(b1)]. Human immunoglobulin allotypes are listed in Table 1 of Jefferis and Lefrance, mAbs 1 (2009), 1-7 and in FIG. 1A of Irani et al., Molecular Immunology 67 (2015), 171-182, which content is herein incorporated by reference. Accordingly, in one embodiment, the antibody as used in accordance with the present invention is of any one of the following allotypes, but not limited thereto: G1m1, G1m2, G1m3, G1m17, G2m23, G3m21, G3m28, G3m11, G3m5, G3m13, G3m14, G3m10, G3m15, G3m16, G3m6, G3m24, G3m26, G3m27, A2 ml, A2m2, A2m3, Em1, Km1, Km2, and Km3, but preferably of G1m2, G1m3, or G1m17, and most preferably of G1m3.

As explained above, antibody NI006/ALXN2220 is a fully human IgG1m3 allotype antibody and composed of two identical heavy chains of the IgG1 subclass and the IgG1m3 allotype. In addition, as mentioned above, original human antibody NI-301.37F1 is of the kappa type and thus, NI006/ALXN2220 is composed of two identical light chains of the kappa subclass. The sequences of the variable heavy (VH) and variable light (VL) chains of NI006/ALXN2220 are set forth in SEQ ID NOs: 2 and 6, and the sequences of the corresponding human constant regions are known in the art. For example, each isotype, such as the IgG1m3 isotype, has a unique amino acid sequence of the constant regions of their heavy chains; see Jefferis and Lefrance (2009), supra. Thus, in one embodiment, the antibody present in the pharmaceutical formulation of the present invention is characterized by two heavy chains, wherein each heavy chain (HC) comprises an amino acid sequence set forth in SEQ ID NO: 9, and by two light chains, wherein each light chain (LC) comprises an amino acid sequence set forth in SEQ ID NO: 10. Each heavy chain is comprised of 450 amino-acid residues, and each light chain consists of 214 amino acid residues. The four chains are stabilized by intra-chain and inter-chain disulfide bonds, wherein the positions of the disulfide bridges, which have been identified per Lys-C and trypsin digestion and subsequent LC-MS (see Example 2) are the following:
LC: C23-LC:C88
LC: C134-LC:C194
LC:C214-HC:C223
HC:C22-HC:C97
HC:C147-HC:C203
HC1:229-HC2:229 and HC1:232-HC2:232
HC:C264-HC:C324
HC:C370-HC:C428.
(Amino acid numbering corresponds to the heavy chain sequence set forth in SEQ ID NO: 9)

Thus, the antibody present in the pharmaceutical composition of the present invention may be characterized to comprise at least 8 disulfide bridges, preferably at the above-identified positions.

Furthermore, each heavy chain of antibody NI006/ALXN2220 contains a single N-linked glycosylation site at Asn300. The N-linked glycosylation structure is predominantly a fucosylated, complex biantennary glycan with 0 galactose residues (GOF) (about 49%) or with 1 galactose residue (G1F) (about 25%). The detailed glycosylation profile is shown in Example 2. Glycosylation plays a vital role in the stability, in vivo activity, solubility, serum half-life and immunogenicity of many therapeutic proteins. N-glycan analysis determines the relative distribution of N-glycans released from the glycoprotein and provides insightful information on the safety and efficacy of bio-therapeutics.

Thus, in a preferred embodiment, the antibody present in the pharmaceutical composition of the present invention is an IgG antibody and has a heavy chain which is N-glycosylated, preferably wherein the N-linked glycosylation site is Asn300, preferably, if for example expressed in CHO cells, wherein the antibody comprises a N-linked glycosylation structure which is predominantly a glycan with 0 galactose residues (GOF) (about 49%) or with 1 galactose residue (G1F) (about 25%). Most preferably, the antibody has the glycosylation profile as shown in Example 3.

Furthermore, one or several amino acids at the amino or carboxy terminus of the light and/or heavy chain, such as the C-terminal lysine of the heavy chain, if present, may be missing or derivatized in a proportion or all of the molecules.

Thus, in one embodiment, the antibody present in the pharmaceutical composition of the present invention has a heavy chain that does not comprise a C-terminal lysine. For example, in such embodiment, the C-terminal lysine included in SEQ ID NO: 9 is missing. The sequence of such a heavy chain is set forth in SEQ ID NO: 13.

In addition, or alternatively, the antibody has a heavy chain, in which the glutamine at the N-terminal is derivatized, preferably substituted with pyroglutamate. This pyroglutamate formation is also referred to as N-terminal cyclization. The sequence of such a heavy chain is set forth in SEQ ID NO: 14 or SEQ ID NO: 15.

Most preferably, the antibody has a heavy chain that does not comprise a C-terminal lysine, i.e., which C-terminal lysine has undergone C-terminal lysine clipping, in which the glutamine at the N-terminal is substituted with pyroglutamate, i.e., which has undergone N-terminal glutaminyl cyclization (see SEQ ID NO: 15), and which is N-glycosylated.

The amino acid sequences of the heavy and light chains are shown below:

QLQLQESGPGLVKPSETLSLTCSVSGGSIISRSSYWGWIRQPPGK
GLEWIGGIYHSGNTYDNPSLKSRLTMSVDTSKNQFSLNLRSVTAA
DTAVYYCARIVPGGDAFDIWGQGTMVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKRVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSREE
MTKNQVSLTCLVKGFYPSDIAVEWESNGQPENNYKTTPPVLDSDG
SFFLYSKLTVDKSRWQQGNVFSCSVMHEALHNHYTQKSLSLSPGK
(SEQ ID NO: 9, NI006/ALXN2220, heavy chain
amino acid sequence, wherein the amino
acids of the constant region are underlined,
and wherein the C-terminal lysine (K) is
optional and/or the N-terminal glutamine
(Q) undergoes intramolecular cyclization,
resulting in the formation of pyroglutamic
acid)
DIQMTQSPSSLSASVGDRVTIACRASQSVGTYLNWYQQKRGKAPK
LLIFAASSLQSGVPSRFSGSGSGTDFTLTISSLQPEDFATYYCQQ
SYSSPPTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
(SEQ ID NO: 10, NI006/ALXN2220, light chain
amino acid sequence, wherein the amino acids
of the constant region are underlined).

Furthermore, the theoretical molecular weight of antibody NI006/ALXN2220 is 144.2 kDa, and the weight determined by mass spectrometry (MS) is 144.2 kDa (deglycosylated) and between 147.0 and 147.6 kDa (intact IgG1), respectively. Thus, in one embodiment, the antibody comprised in the pharmaceutical composition of the present invention has a molecular weight of about 150 kDa, preferably of about 147 kDa.

### Formulations

The present disclosure features pharmaceutical compositions containing the anti-TTR antibody or antigen-binding fragment thereof described above. The pharmaceutical compositions of the present invention may be formulated as described below. For example, a pharmaceutical composition containing the anti-TTR antibody may be formulated to include sucrose, a polysorbate, preferably polysorbate 80, and/or a polar excipient (e.g., histidine). Furthermore, the pharmaceutical composition containing the anti-TTR antibody may be formulated at a desired pH described herein (e.g., pH 5.8). The pharmaceutical composition containing the anti-TTR antibody may further include a pharmaceutically acceptable excipient or diluent, as described herein.

For example, the pharmaceutical composition may include a human anti-TTR antibody or antigen-binding fragment thereof (e.g., an anti-TTR antibody or antigen-binding fragment thereof with a VH region including an amino acid sequence with 100% sequence identity to the amino acid sequence of SEQ ID NO: 7 and a VL region including an amino acid sequence with 100% sequence identity to the amino acid sequence of SEQ ID NO: 8 and an anti-TTR antibody with a heavy chain including an amino acid sequence with 100% sequence identity to the amino acid sequence of SEQ ID NO: 9 and a light chain including an amino acid sequence with 100% sequence identity to the amino acid sequence of SEQ ID NO: 10, respectively) at a concentration of about 50 mg/mL, a histidine buffer with a pH of about 5.8, 6.5% w/v of sucrose, and 0.03% w/v of polysorbate 80 in a volume of 2.0 mL.

In another example, the pharmaceutical composition may include a human anti-TTR antibody or antigen-binding fragment thereof (e.g., an anti-TTR antibody or antigen-binding fragment thereof with a VH region including an amino acid sequence with 100% sequence identity to the amino acid sequence of SEQ ID NO: 7 and a VL region including an amino acid sequence with 100% sequence identity to the amino acid sequence of SEQ ID NO: 8 and an anti-TTR antibody with a heavy chain including an amino acid sequence with 100% sequence identity to the amino acid sequence of SEQ ID NO: 9 and a light chain including an amino acid sequence with 100% sequence identity to the amino acid sequence of SEQ ID NO: 10, respectively) at a concentration of about 50 mg/mL, a histidine buffer with a pH of about 5.8, 8.0% w/v of sucrose, and 0.03% w/v of polysorbate 80 all in a volume of 2.0 mL.

As shown in Example 3, about 99% to 100% of the antibodies present in the pharmaceutical formulation have a N-terminal pyro-glutamic acid in the heavy chain and about 96% of the antibodies have a loss of the C-terminal lysine. Thus, in one embodiment, about 99% of the antibodies in the formulation of the present invention have a heavy chain wherein the N-terminal pyro-glutamic acid is modified from N-terminal glutamine and/or about 96% of the antibodies have a loss of the C-terminal lysine.

Thus, the pharmaceutical composition may include a human anti-TTR antibody or antigen-binding fragment thereof (e.g., an anti-TTR antibody or antigen-binding fragment thereof with a VH region including an amino acid sequence with 100% sequence identity to the amino acid sequence of SEQ ID NO: 11 and a VL region including an amino acid sequence with 100% sequence identity to the amino acid sequence of SEQ ID NO: 8 and an anti-TTR antibody with a heavy chain including an amino acid sequence with 100% sequence identity to the amino acid sequence of SEQ ID NO: 13, 14, or 15, preferably of SEQ ID NO: 15 and a light chain including an amino acid sequence with 100% sequence identity to the amino acid sequence of SEQ ID NO: 10, respectively at a concentration of about 50 mg/mL, a histidine buffer with a pH of about 5.8, 6.5% w/v of sucrose, and 0.03% w/v of polysorbate 80 in a volume of 2.0 mL.

In another example, the pharmaceutical composition may include a human anti-TTR antibody or antigen-binding fragment thereof (e.g., an anti-TTR antibody or antigen-binding fragment thereof with a VH region including an amino acid sequence with 100% sequence identity to the amino acid sequence of SEQ ID NO: 11 and a VL region including an amino acid sequence with 100% sequence identity to the amino acid sequence of SEQ ID NO: 8 and an anti-TTR antibody with a heavy chain including an amino acid sequence with 100% sequence identity to the amino acid sequence of SEQ ID NO: 13, 14, or 15, preferably of SEQ ID NO: 15 and a light chain including an amino acid sequence with 100% sequence identity to the amino acid sequence of SEQ ID NO: 10, respectively at a concentration of about 50 mg/mL, a histidine buffer with a pH of about 5.8, 8.0% w/v of sucrose, and 0.03% w/v of polysorbate 80 all in a volume of 2.0 mL.

In addition, but to a minor extent and preferably in negligible amounts, some antibody species may be found in the analyzed antibody composition that may have undergone other post-translational modifications (PTMs) such as partial cleavage, oxidation, deamidation, succinimide or pyroglutamate formation and isomerization. The PTMs identified to be present in NI006/ALXN2220 are referred to in Example 3. In particular, next to the above-mentioned C-terminal lysine clipping and cyclization of the N-terminus, the antibody may show methionine (M) oxidation, e.g., at HC position 255; asparagine (N) deamidation, e.g., at HC position 318 and/or at HC position 387; asparagine (N) succinimide formation, e.g., at HC position 318; and/or amidation of the C-terminal proline (P) after the loss of the C-terminal lysine and glycine.

### Antibody Concentration

Any of the anti-TTR antibodies or antigen-binding fragments thereof described herein (e.g., the anti-TTR antibody with a VH region with the amino acid sequence of SEQ ID NO: 7 or 11 and a VL region with the amino acid sequence of SEQ ID NO: 8 or 12 and the anti-TTR antibody with a heavy chain with the amino acid sequence of SEQ ID NO: 9 or any one of SEQ ID Nos: 13 to 15 and a light chain with the amino acid sequence of SEQ ID NO: 10, respectively) may be formulated into a pharmaceutic composition described herein at a concentration of about 1 mg/mL to about 500 mg/mL.

The pharmaceutical composition may include about 1 mg/mL to about 400 mg/mL (e.g., from about 1 mg/mL to about 100 mg/mL, e.g., about 1 mg/mL, about 5 mg/mL, about 10 mg/mL, about 25 mg/mL, about 50 mg/mL, about 75 mg/mL, or about 100 mg/mL, e.g., from about 100 mg/mL to about 200 mg/mL, e.g., about 100 mg/mL, about 110 mg/mL, about 125 mg/mL, about 150 mg/mL, about 175 mg/mL, or about 200 mg/mL, e.g., from about 200 mg/mL to about 300 mg/mL, e.g., about 200 mg/mL, about 210 mg/mL, about 225 mg/mL, about 250 mg/mL, about 275 mg/mL, or about 300 mg/mL, e.g., from about 300 mg/mL to about 400 mg/mL, e.g., about 300 mg/mL, about 310 mg/mL, about 325 mg/mL, about 350 mg/mL, about 375 mg/mL, or about 400 mg/mL) of the anti-TTR antibody or antigen binding fragment thereof described herein. The pharmaceutical composition may include about 1 mg/mL to about 300 mg/mL of the anti-TTR antibody or antigen binding fragment thereof described herein. The pharmaceutical composition may include about 1 mg/mL to about 200 mg/mL of the anti-TTR antibody or antigen binding fragment thereof described herein. The pharmaceutical composition may include about 1 mg/mL to about 100 mg/mL (e.g., about 1 mg/mL to about 20 mg/mL, e.g., about 1 mg/mL, about 5 mg/mL, about 10 mg/mL, about 15 mg/mL, or about 20 mg/mL, e.g., about 20 mg/mL to about 40 mg/mL, e.g., about 20 mg/mL, about 25 mg/mL, about 30 mg/mL, about 35 mg/mL, or about 40 mg/mL, about 40 mg/mL to about 60 mg/mL, e.g., about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, or about 60 mg/mL, e.g., about 60 mg/mL to about 80 mg/mL, e.g., about 60 mg/mL, about 65 mg/mL, about 70 mg/mL, about 75 mg/mL, or about 80 mg/mL, e.g., about 80 mg/mL to about 100 mg/mL, e.g., about 80 mg/mL, about 85 mg/mL, about 90 mg/mL, about 95 mg/mL, or about 100 mg/mL) of the anti-TTR antibody or antigen binding fragment thereof described herein. The pharmaceutical composition may include about 30 mg/mL to about 70 mg/mL of the anti-TTR antibody or antigen binding fragment thereof described herein. The pharmaceutical composition may include about 40 mg/mL to about 60 mg/mL (e.g., about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, or about 60 mg/mL) of the anti-TTR antibody or antigen binding fragment thereof described herein.

The pharmaceutical composition may include about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL of the anti-TTR antibody or antigen binding fragment thereof described herein. The pharmaceutical composition may include about 50 mg/mL of the anti-TTR antibody or antigen binding fragment thereof described herein.

### Sucrose

The pharmaceutical composition may also include sucrose, for example, in an amount of about 6% to about 9%, about 6% to about 7%, or about 7.5% to about 8.5% weight per unit volume (w/v) (e.g., about 6%, 6.5%, 7%, 7.5%, 8%, 8.5%, or 9% w/v sucrose). For example, the pharmaceutical composition may include about 6%, 6.1%, 6.2%, 6.3%, 6.4%, 6.45%, 6.5%, 6.55%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.05%, 8.1%, 8.2%, 8.3%, 8.4%, or 8.5% w/v sucrose. In particular, the pharmaceutical composition includes about 6.5% w/v sucrose or about 8% w/v sucrose.

### Polysorbate

The pharmaceutical composition may also include a polysorbate for example polysorbate 20 or polysorbate 80, preferably polysorbate 80 (PS80), for example, in an amount of about 0.001% to about 0.1% w/v (e.g., about 0.001%, 0.005%, 0.01%, 0.05% or 0.1% w/v PS(80)). For example, the pharmaceutical composition may include about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1% w/v PS80. In particular, the pharmaceutical composition includes about 0.03% w/v PS(80).

### Polar Excipients/Buffer System

The pharmaceutical composition may also include a polar excipient. The polar excipient may be or include, for example, a sugar, a polyol, or an amino acid. The sugar may be, for example, sucrose, trehalose, fructose, lactose, dextrose, or mannitol. The polyol may be, for example, polyethylene glycol or sorbitol. The amino acid may be, for example, one or more of alanine, arginine, aspartic acid, asparagine, carnitine, citrulline, ornithine, glycine, glutamic acid, glutamine, glycine, histidine, isoleucine, leucine, lysine, methionine, phenylalanine, proline, serine, threonine, tyrosine, and valine. In a preferred embodiment, the polar excipient, which is used as buffering agent in the formulation of the present invention, is histidine (e.g., L-histidine and/or L-histidine monohydrochloride, or a pharmaceutically acceptable salt thereof). In some embodiments, the polar excipient is L-histidine and/or L-histidine monohydrochloride, or a pharmaceutically acceptable salt thereof. As known by the skilled person, polar excipients comprise buffer agents and are known in the art, and include for example citrate buffers, phosphate buffers, acetate buffers, histidine buffers and combinations thereof. Suitable buffers can be chosen especially using the guidance provided above. The preferred buffer for use in accordance with the present invention is a histidine buffer. The pharmaceutical composition may include a polar excipient (buffer) (e.g., histidine) in an amount of about, for example, about 1 mM to about 100 mM (e.g., about 10 mM, 20 mM, 30 mM, 40 mM, 50 mM, 60 mM, 70 mM, 80 mM, mM, 90 mM, or 100 mM). For example, the pharmaceutical may include about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 19.5 mM, about 20 mM, about 20.5 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, or about 100 mM, of a polar excipient (e.g., histidine). In particular, the pharmaceutical composition includes about 20 mM of a polar excipient (e.g., histidine).

pH

The pharmaceutical composition may have a pH of from about 5.0 to about 8.0 (e.g., about 5.5, 6.0, 6.5, 7.0, 7.5, or 8.0). For example, the pharmaceutical composition may have a pH of about 5.0, 5.1, 5.2, 5.3, 5.4, 5.5, 5.6, 5.7, 5.75, 5.8, 5.85, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In particular, the pharmaceutical composition has a pH of about 5.8.

### Excipients and Diluents

The pharmaceutical composition may contain a pharmaceutically acceptable excipient (e.g., a buffer, carrier, stabilizer, or preservative) or diluent (e.g., saline and aqueous buffer solutions). Pharmaceutically acceptable buffers include, e.g., bacteriostatic water for injection (BWFI), phosphate-buffered saline (PBS), Ringer's solution, and dextrose solution.

### Volumes

The pharmaceutical composition may be provided (e.g., in a vial or other container, as described herein) in a volume of about 1 mL to about 100 mL, about 1 mL to about 50 mL, about 5 mL to about 25 mL, about 20 mL to about 25 mL, or about 1 mL to about 10 mL (e.g., about 1 mL to about 80 mL, about 1 mL to about 70 mL, about 1 mL to about 60 mL, about 1 mL to about 50 mL, about 1 mL to about 40 mL, about 1 mL to about 30 mL, about 1 mL to about 20 mL, about 1 mL to about 10 mL, about 5 mL to about 20 mL, about 5 mL to about 15 mL, about 5 mL to about 10 mL, about 10 mL to about 20 mL, about 15 mL to about 20 mL, about 20 mL to about 22.5 mL, about 20 mL to about 25 mL, about 1 mL to about 9 mL, about 1 mL to about 8 mL, about 1 mL to about 7 mL, about 1 mL to about 6 mL, about 1 mL to about 5 mL, about 1 mL to about 4 mL, about 1 mL to about 3 mL, about 1 mL to about 2.25 mL, about 1 mL to about 2 mL, or about 2 mL to about 2.25 mL). For example, the pharmaceutical composition may be present in a volume of about 1 mL to about 2.25 mL (e.g., about 1 mL to about 2.2 mL, about 1 mL to about 2 mL, about 1 mL to about 1.8 mL, about 1 mL to about 1.6 mL, about 1 mL to about 1.4 mL, about 1 mL to about 1.2 mL, about 1.5 mL to about 1.25 mL, about 1.5 mL to about 2 mL, about 1.9 mL to about 1.2 mL, about 2.1 mL to about 2.25 mL) or about 1 mL to about 100 mL (e.g., about 1 mL, about 1.8 mL, about 1.9 mL, about 2 mL, about 2.1 mL, about 2.2 mL about 2.25 mL, about 2.3 mL, about 2.4 mL, about 2.5 mL about 3 mL, about 4 mL, about 5 mL, about 6 mL, about 7 mL, about 8 mL, about 9 mL, about 10 mL, about 20 mL, about 30 mL, about 40 mL, about 50 mL, about 60 mL, about 70 mL, about 80 mL, about 90 mL, or about 100 mL).

In a specific example, the pharmaceutical composition may be present in a volume of about 2.25 mL or about 2 mL. In another example, the pharmaceutical composition may be present in a volume of about 20 mL or about 22.5 mL.

### Additional Formulations

The pharmaceutical composition may be prepared so that it has not been reconstituted from a lyophilized anti-TTR antibody and/or is not further lyophilized. Also, the pharmaceutical composition may be prepared so that it is essentially free of sodium chloride and/or essentially free of a poloxamer. The pharmaceutical composition can also be prepared as a sterile composition.

In the most preferred embodiment of the present invention, the pharmaceutical composition comprises or consist of 50 mg/mL of the antibody (antibody NI006/ALXN2220 as characterized hereinbefore) 20 mM histidine (L-Histidine 1.06 mg/mL and L-Histidine monohydrochloride 2.78 mg/mL), 65 mg/mL or 80 mg/mL sucrose, preferably 80 mg/mL sucrose, 0.3 mg/mL polysorbate 80, and water for infusion/injection at pH 5.8.

### Characteristics

The disclosure features the invention of a pharmaceutical composition with improved characteristics (e.g., stability, solubility, storage, etc.), as described herein (e.g., see Examples 1 and 2).

The pharmaceutical composition can be characterized by having a shelf-life of 24 months at 2 to 8° C., preferably when protected from light.

The pharmaceutical composition can be characterized by one, two, three or all four of the following stability criteria (e.g., (a) to (d)) in any combination: (a) the main peak decline under heat stress conditions for 4 weeks at 40° C. and/or 12 weeks at about 25° C. is less than 1% of antibody by weight as measured by Size Exclusion Chromatography (SEC)-HPLC analysis; (b) the pharmaceutical composition shows a content of acidic species of the anti-TTR antibody under heat stress conditions for 2 weeks at about 40° C. of less than 42.5% as measured by Capillary Isoelectric Focusing (cIEF); (c) the pharmaceutical composition shows no substantial change in the content of acidic species of the anti-TTR antibody after 3 cycles freeze thaw (about −70° C. to RT (e.g., 20-26° C., such as 25° C.) as measured by Capillary Isoelectric Focusing (cIEF); and/or (d) the anti-TTR antibody retains a binding potency to a TTR protein of at least 80% after storage for 4 weeks at 40° C. and/or a binding potency to a TTR protein of at least 70% after storage for 12 weeks at 25° C., e.g., as measured by ELISA and relative to a control (e.g., without prolonged storage).

For example, the pharmaceutical composition shows a main peak≥50.0%, an acidic peak≤40.0%, and a basic peak≤15.0% as measured, e.g., by cIEF, a main peak (monomer)≥95.0% and High Molecular Weight Species (HMWS)≤5.0% as measured by, e.g., Size Exclusion Chromatography (SEC)-HPLC analysis, a pH 5.8±0.5, an osmolality 240 mOsm/Kg, and an antibody concentration of 50±5.0 mg/mL.

In addition, the pharmaceutical composition of the present invention may be characterized by one, two, three or all four stability criteria (i) to (iv) in any combination: (i) the main peak decline (representing the monomer content) under heat stress conditions for 1 month at about 40° C., or for 6 months at about 25° C., or during long-term storage for 18 months at about 5° C. is less than 5%, preferably less than 4%, more preferably less than 3%, more preferably less than 2% as measured by SEC-HPLC; (ii) the pharmaceutical composition shows a content of acidic species of the anti-TTR antibody or antigen-binding fragment thereof under heat stress conditions for 2 weeks at about 40° C., or for 3 months at about 25° C. of less than or about equal to about 40% as measured by cIEF; (iii) the pharmaceutical composition shows a content of acidic species of the anti-TTR antibody or antigen-binding fragment thereof under long-term storage conditions for 12 months or 18 months at about 5° C. of less than 40%, preferably of less than 35% as measured by cIEF; and/or (iv) the anti-TTR antibody or antigen-binding fragment thereof retains binding potency to a TTR protein of at least 80%, preferably of at least 90% after storage for 6 months at about 25° C., or after storage for 12 months or 18 months at about 5° C. as measured by ELISA and relative to a control (e.g., without prolonged storage).

Preferably, the pharmaceutical composition shows any one or all of the characteristics as set forth in Tables 56 to 69 of the Examples.

In particular, the pharmaceutical composition has an osmolality of ≥240 mOsm/Kg and includes sucrose (e.g., at about 6.5% or about 8% (w/v), and optionally a surfactant (e.g., PS80 in an amount of about 0.03% w/v)).

The pharmaceutical composition is stable upon freezing and thawing. As used herein, the term “stable” or “stability”, as used in the context of a pharmaceutical composition described herein, refers to the maintenance of the physical and functional characteristics of the composition over time. For example, a stable composition may be described as one that retains its appearance (e.g., color, opalescence, number of visible particles, and/or number of subvisible particles), pH, antibody concentration, and/or osmolarity after long term storage (e.g., days or weeks), after storage at various temperatures (e.g., −70° C., 4° C., 25° C., or 40° C.), and/or after one or more (e.g., 1, 2, 3, 4, 5, or more) freeze-thaw cycles. As another example, a stable composition may be described as one that retains its function (e.g., anti-TTR binding ability, e.g., as described herein) after storage at various temperatures (e.g., −70° C., 4° C., 25° C., or 40° C.), and/or after one or more (e.g., 1, 2, 3, 4, 5, or more) freeze-thaw cycles. The pharmaceutical composition of the present invention has been shown to remain stable at 40±2° C. and 75±5% RH for at least 1 month (stress stability studies); at 25±2° C./60±5% RH for at least 6 months (accelerated stability studies); and/or at 5±3° C. for at least 12 months to 18 months (long-term stability studies). Furthermore, the extinction coefficient of the pharmaceutical composition has been determined to be 1.438 (mg/mL)¹ cm⁻¹ and the pharmaceutical formulation has been shown to be a sterile, colorless to slightly yellow, clear to slightly opalescent solution, essentially free of visible particles with a pH of 5.8.

### Methods of Treatment

The pharmaceutical composition containing the anti-TTR antibody or antigen-binding fragment thereof (e.g., an anti-TTR antibody or antigen-binding fragment thereof with a VH region including an amino acid sequence with at least 80% sequence identity to the amino acid sequence of SEQ ID NO: 7 (e.g., at least 85%, 90%, 95%, 97%, 99%, or 100% sequence identity to SEQ ID NO: 7, or 100% sequence identity to SEQ ID NO: 11, and, e.g., also having the CDR sequences set forth in SEQ ID NOs: 1-3) and a VL region including an amino acid sequence with at least 80% sequence identity sequence identity to the amino acid sequence of SEQ ID NO: 8 (e.g., at least 85%, 90%, 95%, 97%, 99%, or 100% sequence identity to the amino acid sequence of SEQ ID NO: 8, and, e.g., having the CDR sequences set forth in SEQ ID NOs: 4-6) and an anti-TTR antibody with a heavy chain including an amino acid sequence with 100% sequence identity to the amino acid sequence of SEQ ID NO: 9, preferably including the PTMs mentioned hereinbefore, i.e. an anti-TTR antibody with a heavy chain including an amino acid sequence with 100% sequence identity to the amino acid sequence of SEQ ID NO: 13, 14, or 15, preferably of SEQ ID NO: 15, and a light chain including an amino acid sequence with 100% sequence identity to the amino acid sequence of SEQ ID NO: 10, respectively) can be administered to a subject (e.g., a human) to treat, prevent, or control a disease or disorder as described herein. For example, the pharmaceutical compositions described herein can be used in a method of treating or preventing transthyretin-mediated amyloidosis (ATTR). Additionally, the pharmaceutical compositions described herein can be used in a method of treating or preventing ATTR amyloidosis with cardiomyopathy (ATTR-CM, such as WT-ATTR-CM). For example, a pharmaceutical composition containing a human anti-TTR antibody or antigen-binding fragment thereof (e.g., an anti-TTR antibody or antigen-binding fragment thereof with a VH region including an amino acid sequence with 100% sequence identity to the amino acid sequence of SEQ ID NO: 7 or 100% sequence identity to SEQ ID NO: 11, and a VL region including an amino acid sequence with 100% sequence identity to the amino acid sequence of SEQ ID NO: 8 and an anti-TTR antibody with a heavy chain including an amino acid sequence with 100% sequence identity to the amino acid sequence of SEQ ID NO: 9, preferably including the PTMs mentioned hereinbefore, i.e. an anti-TTR antibody with a heavy chain including an amino acid sequence with 100% sequence identity to the amino acid sequence of SEQ ID NO: 13, 14, or 15, preferably of SEQ ID NO: 15, and a light chain including an amino acid sequence with 100% sequence identity to the amino acid sequence of SEQ ID NO: 10, respectively) at a concentration of about 50 mg/mL, a histidine buffer with a pH of about 5.8, 6.5% or 8.0% w/v of sucrose, and 0.03% w/v of polysorbate 80 can be administered to treat ATTR or ATTR-CM, such as WT-ATTR-CM.

The pharmaceutical composition containing the anti-TTR antibody can be administered to a human subject to treat, prevent, or control transthyretin-mediated amyloidosis (ATTR), including ATTR amyloidosis with cardiomyopathy (ATTR-CM, such as WT-ATTR-CM). The pharmaceutical composition can be administered by intravenous injection or infusion at a dose providing 30-60 mg/kg (e.g., 30 mg/kg, 35 mg/kg, 40 mg/kg, 45 mg/kg, 50 mg/kg, 55 mg/kg, or 60 mg/kg) of the human anti-TTR antibody or up to 75000 mg (e.g., 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, or 7500 mg) of the human anti-TTR antibody to the subject. For example, a dose of 3000 mg or a dose of 7500 mg, as well as doses in between, can be used to treat a human subject.

The composition and methods provided herein may be used to treat a subject that has ATTR, ATTR-CM, ATTR polyneuropathy (ATTR-PN), Familial Amyloid Polyneuropathy (FAP), Familial Amyloid Cardiomyopathy (FAC), Senile Systemic Amyloidosis (SSA), systemic familial amyloidosis, leptomeningeal/Central Nervous System (CNS) amyloidosis, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, preeclampsia, or a known pathogenic TTR mutation (e.g., one that causes amyloidosis). The subject may have sporadic, WT-ATTR-CM and a negative genetic testing for a TTR mutation.

A pharmaceutical composition of the present disclosure can be formulated for administration by a variety of methods known in the art. Administration may be, for example, intravenous or subcutaneous. Intravenous delivery by continuous infusion is one method for administering the pharmaceutical compositions disclosed herein.

The pharmaceutical composition may be ready-to use for administration to a subject in need thereof, preferably via intravenous infusion. The pharmaceutical composition may be diluted prior to infusion with glucose or a polymer thereof, preferably wherein the polymer is dextran. The concentration of glucose or a polymer thereof may be 1%, 2%, 3%, 4%, 5%, 6%, 7%, 8%, 9%, or 10% w/v.

In some instances, the pharmaceutical composition is administered in a dosing regimen described in ClinicalTrials.gov ID NCT04360434 and Garcia-Pavia et al, (2023) and European patent application EP 22 207 651.5 and EP 23 020 175.8; U.S. Patent Application No. 63/383,807; and international application PCT/EP2023/081809 filed on Nov. 15, 2023, claiming priority from EP 22 207 651.5, EP 23 020 175.8 and U.S. 63/383,807 (herein incorporated by reference). Accordingly, in a preferred embodiment, the pharmaceutical composition of the present invention, especially the most preferred composition of NI006/ALXN2220 referenced above is administered about every 28 days.

### Articles of Manufacture

The present disclosure also features an article of manufacture (e.g., a kit) containing materials useful for the treatment or prevention of transthyretin-mediated amyloidosis (ATTR) in a human subject. The article of manufacture includes one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 7, 48, 49 50, or 50 or more) containers and a label or package insert on or associated with the one or more containers. Suitable containers include, for example, bottles (e.g., infusion bottles), vials (e.g., a Type I clear glass vial), syringes, IV solution bags, etc. Containers, such as Type I clear glass vials or infusion bottles, may be 1 mL, 2 mL, 5 mL, 10 mL, 15 mL, 20 mL, or 25 mL in size and capable of accommodating about a 10% to about 15% (e.g., 10% or 12.5%) volume overfill of the pharmaceutical composition. The container may include a volume of a pharmaceutical composition described herein, such as a volume of, e.g., 0.5 mL to 10 mL, 2 mL to 2.25 mL, 10 mL to 20 mL, 15 mL to 25 mL, 20 mL to 22.5 mL, e.g., 0.5 mL, 1 mL, 2 mL, 2.25 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 20.5 mL, 21 mL, 21.5 mL, 22 mL, 22.5 mL, 23 mL, 24 mL, or 25 mL. In a preferred embodiment, the pharmaceutical composition, i.e., the drug product described herein is provided as a concentrate for solution for infusion that is presented as sterile, colorless to slightly yellow, clear to slightly opalescent liquid, essentially free of visible particles and supplied in a 2 mL (2R) glass vial with an aluminum flip-off cap over a 13 mm rubber stopper. The product is preferably diluted in sterile glucose before administration, which is a commercial product and in one embodiment not accompanying the drug product.

The pharmaceutical composition may contain, e.g., a human anti-TTR antibody or antigen-binding fragment thereof (e.g., an anti-TTR antibody or antigen-binding fragment thereof with a VH region including an amino acid sequence with 100% sequence identity to the amino acid sequence of SEQ ID NO: 7 or SEQ ID NO: 11, and a VL region including an amino acid sequence with 100% sequence identity to the amino acid sequence of SEQ ID NO: 8, and an anti-TTR antibody with a heavy chain including an amino acid sequence with 100% sequence identity to the amino acid sequence of SEQ ID NO: 9, preferably including the PTMs mentioned hereinbefore, i.e. an anti-TTR antibody with a heavy chain including an amino acid sequence with 100% sequence identity to the amino acid sequence of SEQ ID NO: 13, 14, or 15, preferably of SEQ ID NO: 15, and a light chain including an amino acid sequence with 100% sequence identity to the amino acid sequence of SEQ ID NO: 10, respectively) at a concentration of about 1 mg/mL to about 150 mg/mL (e.g., about 50 mg/mL, about 60 mg/mL, 70 mg/mL, about 80 mg/mL, 90 mg/mL, about 100 mg/mL, about 110 mg/mL, about 120 mg/mL, about 130 mg/mL, about 140 mg/mL, or about 150 mg/mL), and with, e.g., a histidine buffer with a pH of about 5.8, 6.5% or 8.0% w/v of sucrose, and 0.03% w/v of polysorbate 80. The pharmaceutical composition of the article of manufacture may contain, e.g., an anti-TTR antibody or antigen-binding fragment thereof with a VH region including an amino acid sequence with at least 80% sequence identity to the amino acid sequence of SEQ ID NO: 7 (e.g., at least 85%, 90%, 95%, 97%, 99%, or 100% sequence identity to the amino acid sequence of SEQ ID NO: 7 or SEQ ID NO: 11, and, e.g., having the CDR sequences set forth in SEQ ID NOs: 1-3) and a VL region including an amino acid sequence with at least 80% sequence identity to the amino acid sequence of SEQ ID NO: 8 (e.g., at least 85%, 90%, 95%, 97%, 99%, or 100% sequence identity to the amino acid sequence of SEQ ID NO: 8, and, e.g., having the CDR sequences set forth in SEQ ID NOs: 4-6, and the anti-TTR antibody with a heavy chain with the amino acid sequence of SEQ ID NO: 9, preferably including the PTMs mentioned hereinbefore, i.e. an anti-TTR antibody with a heavy chain including an amino acid sequence with 100% sequence identity to the amino acid sequence of SEQ ID NO: 13, 14, or 15, preferably of SEQ ID NO: 15, and a light chain with the amino acid sequence of SEQ ID NO: 10, respectively) at a total amount of about 2500 mg to about 7500 mg (e.g., about 2500 mg to about 3000 mg, about 2750 mg to about 3500 mg, about 3000 mg to about 4000 mg, about 3500 mg to about 4500 mg, about 4000 mg to about 5000 mg, about 4500 mg to about 5500 mg, about 5000 mg to about 6000 mg, about 5500 mg to about 6500 mg, about 6000 mg to about 7000 mg, or about 6500 mg to about 7500 mg, e.g., 2500 mg, 2750 mg, 3000 mg, 3250 mg, 3500 mg, 3750 mg, 4000 mg, 4250 mg, 4500 mg, 4750 mg, 5000 mg, 5250 mg, 5500 mg, 5750 mg, 6000 mg, 6250 mg, 6500 mg, 6750 mg, 7000 mg, 7250 mg, or 7500 mg) and with, e.g., a histidine buffer with a pH of about 5.8, 6.5% or 8.0% w/v of sucrose, and 0.03% w/v of polysorbate 80. The pharmaceutical composition of the article of manufacture may contain a total amount of about 2500 mg to about 5000 mg (e.g., 2500 mg to 3000 mg, 2750 mg to about 3500 mg, 3000 mg to about 4000 mg, 3500 mg to 4500 mg, or 4000 mg to 5000 mg, e.g., 2500 mg, 2750 mg, 3000 mg, 3250 mg, 3500 mg, 3750 mg, 4000 mg, 4250 mg, 4500 mg, 4750 mg, or 5000 mg) of the anti-TTR antibody or antigen-binding fragment thereof (e.g., an anti-TTR antibody or antigen-binding fragment thereof with a VH region including an amino acid sequence with at least 80% sequence identity to the amino acid sequence of SEQ ID NO: 7 (e.g., at least 85%, 90%, 95%, 97%, 99%, or 100% sequence identity to the amino acid sequence of SEQ ID NO: 7 or SEQ ID NO: 11, and, e.g., having the CDR sequences set forth in SEQ ID NOs: 1-3) and a VL region including an amino acid sequence with at least 80% sequence identity to the amino acid sequence of SEQ ID NO: 8 (e.g., at least 85%, 90%, 95%, 97%, 99%, or 100% sequence identity to the amino acid sequence of SEQ ID NO: 8, and, e.g., having the CDR sequences set forth in SEQ ID NOs: 4-6, and the anti-TTR antibody with a heavy chain with the amino acid sequence of SEQ ID NO: 9 and a light chain with the amino acid sequence of SEQ ID NO: 10) in a total volume of about 2 mL to about 25 mL (e.g. 2 mL, 2.25 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 20.5 mL, 21 mL, 21.5 mL, 22 mL, 22.5 mL, 23 mL, 24 mL, or 25 mL).

The containers may be formed from a variety of materials such as glass or plastic. The container can be one that holds a pharmaceutical composition described herein, which is by itself or combined with another composition effective for treating or preventing ATTR. The container may have a sterile access port (for example, the container may be an intravenous solution bag or a vial having a stopper pierceable by a hypodermic injection needle). At least one active agent in the composition is an anti-TTR antibody described herein. The label or package insert indicates that the composition is used for treating ATTR.

The article of manufacture may include at least a first container with a pharmaceutical composition contained therein, in which the pharmaceutical composition includes an anti-TTR antibody described herein. Optionally, the article of manufacture may further include a second container with a second therapeutic agent. The article of manufacture in this embodiment of the disclosure may further include a package insert indicating that the compositions can be used to treat ATTR. Alternatively, or additionally, the article of manufacture may further include a second (or third) container including a pharmaceutically acceptable buffer, such as BWFI, PBS, Ringer's solution, and dextrose solution.

The article of manufacture may further include other materials desirable from a commercial and user standpoint, including other buffers, diluents, filters, needles, and syringes. For example, the article of manufacture (e.g., a kit) may include a second container with a pharmaceutically acceptable buffer, such as phosphate-buffered saline, Ringer's solution, and/or a glucose or a polymer thereof, such as dextran (e.g., at a concentration of about 5% w/v). The article of manufacture may include other materials, including buffers, diluents, filters, needles, syringes, and package inserts with instructions for use.

In a preferred embodiment, the article of manufacture includes vials, preferably clear glass vials sealed with a (grey) rubber stopper and a (blue) aluminium-plastic cover flip-off cap. Preferably, the antibody is presented in vials at a concentration of 50 mg/mL and is provided as a concentrate for solution for infusion that is presented as sterile, colorless to slightly yellow, clear to slightly opalescent liquid, essentially free of visible particles.

In a further preferred embodiment, the article of manufacture includes a dosing syringe in a dosing pump or an infusion bag comprising the diluted antibody formulation.

### EXAMPLES

The following examples of specific aspects for carrying out the present disclosure are offered for illustrative purposes only and are not intended to limit the scope of the present disclosure in any way.

### Example 1. Formulation Development of Anti-TTR Monoclonal Antibody

This example summarizes the results of the formulation development of an anti-transthyretin (TTR) monoclonal antibody (mAb) having the heavy chain having an amino acid sequence of SEQ ID NO: 9 and a light chain having an amino acid sequence of SEQ ID NO: 10 and obtained by recombinant expression in CHO-K1 cells, respectively. The physical and chemical stability of the anti-TTR antibody was evaluated to design a stable formulation for clinical development. Experiments were aimed at evaluating the effect of excipients on the antibody properties under accelerated and stressed conditions. Biophysical and analytical methods were used to determine the potential chemical and physical changes that occurred to the formulated antibody under these various conditions.

A goal of the formulation development study was to identify a stable formulation (e.g., pharmaceutical composition) for the antibody, as determined according to a pH buffer study, solubility study, excipients screening study and surfactant screening study.

### Analytical Methods

### Appearance

The appearance of all samples, including clarity, color and visible particles, was examined against black and white background using YB-2 light box.

### Caliper-SDS R/NR

Caliper-sodium dodecyl sulfate (SDS) reduced (R)/non-reduced (NR) was performed on a PerkinElmer Caliper automated electrophoresis. Sample was treated with sample buffer, SDS and N-ethylmaleimide (for non-reduced or NR) or dithiothreitol (for reduced or R) at 70° C. for 10 min. Then the sample with a minimum volume of 42 μL (final antibody concentration of 0.045 mg/ml) was tested by LABCHIP® GXII TOUCH™ at excitation/emission wavelength of 635 and 700 nm. The final results were analyzed by the commercial software: LABCHIP® GX Reviewer.

cIEF

The capillary isoelectric focusing (cIEF) was performed on a PROTEINSIMPLE® cIEF analyzer equipped with FC-coated cIEF cartridge. Fifty μg of each sample was mixed with 90 μl of master mix which consisted of isoelectric point (pI) marker 7.65/9.46, PHARMALYTE™ 3-10, PHARMALYTE™ 8-10.5, 1% methylcellulose, and 8M urea. After mixing, the sample was focused 1 minute at 1500 V and 8 minutes at 3000 V, respectively. Detection wavelength was set at 280 nm to evaluate the charge variants distribution in different pI ranges.

### DLS

Zeta potential was measured by Malvern dynamic light scattering (DLS). Samples were diluted to 5 mg/mL, and 1 mL of each sample was placed into the DLS sample cell.

### ELISA Binding

This assay was an enzyme-linked immunosorbent assay (ELISA) method for the binding potency of the antibody. Samples and reference standard at appropriate dilutions were loaded onto misfolded-TTR coated half-area 96 well plate(s). After washing, the antibody product is added to the wells. After washing the plate, HRP-conjugated goat anti-human IgG are added to the wells allowing interaction with the antibody captured during the previous step. After a final wash step, TMB substrate solution (Cat. N301, THERMO SCIENTIFIC™) is loaded into the wells. TMB specifically reacts with the peroxide in the presence of peroxidase and produces a colorimetric signal that is proportional to the amount of the antibody bound to the wells. The color development is stopped, and optical density is measured at 450/560 nm. Dose response curves are plotted according to a 4 parameter logistic (Auto-Estimate) regression model using the SOFTMAX® Pro Software for samples, control, and reference standard. Individual EC50 was calculated for the sample and reference standard, respectively. Relative binding activity of the sample was calculated using the EC50 of reference to samples on the same plate.

### Osmolality

Osmolality was measured using an Osmometer. Before and after testing, method accuracy of the osmometer was confirmed with a clinitrol 290 milliosmoles (mOsm) reference solution. The sample volume for testing was 20 μL and only one test was performed for each sample.

### MFI

Subvisible particles were monitored by a microfluidic imaging (MFI) system. About 1.5 ml volume of each sample was transferred to the MFI 96-well plate in bio-safety hood for analysis. The results were analyzed by the vendor's software. The subvisible particle amount in the equivalent circular diameter between over 2 μm, over 10 μm, and over 25 μm was reported.

### Particulate Matter (High Accuracy (HIAC))

A HACH Particulate Analyzer was utilized to measure the subvisible particle size and counts under a laminar flow cabinet. To avoid introducing air bubbles and interference during examination, all samples were held in the cabinet for at least 0.5 hour before testing. Each sample was tested for four consecutive runs, 0.45 mL each. The results were presented as average number of particles of ≥10 μm and ≥25 μm per mL (method conforms to USP <788> Particulate matter in injections).

pH

Sample pH was measured using a pH meter with glass electrode. The pH meter was calibrated prior to use each time.

### Antibody Concentration

Antibody concentration was determined by a THERMO™ UV spectrophotometer. The extinction coefficient used in all evaluation studies was 1.390 AU*mL*mg⁻¹*cm⁻¹. All measurements were repeated twice with 2.5 μL sample each time and an average result was reported. For concentration over 100 mg/mL, samples were diluted to 20 mg/mL before testing.

### SEC-HPLC

Size exclusion chromatography (SEC) was performed on an AGILENT® high performance liquid chromatography (HPLC) system with a SEC column (300×7.8 mm, 5 μm). The sampler temperature was set to 5±3° C. and the column oven temperature was set as 25±3° C. The mobile phase was 50 mM PB, 300 mM NaCl, pH 6.8±0.1 and the flow rate was set as 1.0 mL/min. Samples were diluted to 10 mg/mL with mobile phase and 100 μg samples were injected. Detection wavelength was set at 280 nm and the run time was 20 minutes.

### Turbidity (UV350)

Turbidity was performed by a spectrophotometer (SPECTRA MAX™). One-hundred and fifty μL samples were added into the wells of a 96-well plate, and 150 μL of respective buffers were also added into the corresponding wells as the reference. Then the absorption of the buffers and samples were tested at 350 nm. The UV350 value of the antibody was obtained by subtracting the corresponding buffer.

### Viscosity

Viscosity was measured by a viscometer (Brookfield), and a CP-51 cone and plate geometry was used. About 0.5 mL sample was used for each measurement, and before sample measurement, a Brookfield viscosity standard was used (29.14 cP).

### Pre-Formulation Study

A pH/buffer screening study and a solubility study was performed, as detailed below, in order to identify an optimal formulation (e.g., pharmaceutical composition) for the anti-TTR antibody.

pH/Buffer Screening Study

This study aimed to screen out the most suitable pH buffer condition for the anti-TTR antibody.

### Sample Preparation

Nine candidate buffers with different pH were used in this study and the details of these buffers are shown in Table 2. Three buffer systems were used: 20 mM acetate, 20 mM histidine and 20 mM phosphate buffer (PB), with pH value ranging from 4.5 to 7.5. The drug substance (DS) (formulated in 20 mM histidine, pH 6.0) generated from a 50 L pool was used for this study. The DS was buffer exchanged into the 3 prepared buffers (20 mM acetate pH 5.0, 20 mM histidine pH 6.0, and 20 mM PB pH 7.0) by dialysis. Six other pH buffer conditions were achieved by adjusting the solution using a corresponding acid or base. The anti-TTR antibody concentration used in this study was 50 mg/mL. Each of the prepared samples was filtered and filled into 2 ml glass vial (1 ml/vial), then stoppered, capped, and labeled immediately.

TABLE 2
Buffer candidates list for pH screening

| Formulation | | | Antibody |
|---|---|---|---|
| No. | Buffer System | pH | Concentration |
| F1 | 20 mM acetate | 4.5 | 50 mg/ml |
| F2 | buffer | 5.0 | 50 mg/ml |
| F3 | | 5.5 | 50 mg/ml |
| F4 | 20 mM histidine | 5.5 | 50 mg/ml |
| F5 | buffer | 6.0 | 50 mg/ml |
| F6 | | 6.5 | 50 mg/ml |
| F7 | 20 mM phosphate | 6.5 | 50 mg/ml |
| F8 | buffer | 7.0 | 50 mg/ml |
| F9 | | 7.5 | 50 mg/ml |

### Study Parameters

Table 2 shows the evaluated condition for the pH/buffer screening study. Samples were stored at 40° C. for 4 weeks. Samples were retrieved timely at each time point and kept at 2-8° C. before analysis. Testing items including appearance, pH, antibody concentration, SEC-HPLC, cIEF, Caliper-SDS (non-reduced (NR) and reduced (R)), dissociation constant (kD) value by DLS, and zeta potential were performed for this study, as detailed further below. For a selected sample F6 at a 4 week (w) time point, ELISA binding antigen was tested.

TABLE 3
Study parameters of pH screening study

| Formulation | | Time Point | | |
|---|---|---|---|---|
| No. | Time 0 | 1 w | 2 w | 4 w |
| F1-F9 | X, Y | X | X | X, Z |

X = appearance, pH, antibody concentration (A280), SEC-HPLC, cIEF, Caliper-SDS (NR & R);
Y = kD value (DLS), zeta potential;
Z = Elisa binding potency (only for F6);
w = week(s)

Results of the pH/Buffer Screening Study

### Appearance, pH, and Protein Concentration

Data summary of appearance, pH and antibody concentration in pH screening study is listed in Table 4.

For samples in acetate or histidine buffer (F1-F6), samples showed light yellow, slightly opalescent with visible particles at T0, and remained similar appearance after four weeks incubation at 40° C. Among these six samples, F2 (20 mM acetate pH 5.0) showed a few particles at T0, but later showed no visible particles after incubation at 40° C. For F6 (20 mM histidine pH 6.5), a few particles were observed at T0, and large number of particles were observed at the 4w point.

In these 6 samples, the appearance of the samples was closely related with the pH value. As the pH increased, more particles were observed, and the sample became more turbid in appearance.

For phosphate buffer samples (F7-F9), opalescent liquid with a large amount of particles were observed from T0, and the appearance remained after four week incubation at 4000.

TABLE 4
Data summary of appearance, pH, and antibody concentration in pH screening study

| Formulation No. | | F1 | F2 | F3 | F4 | F5 | F6 | F7 | F8 | F9 |
|---|---|---|---|---|---|---|---|---|---|---|
| Appearance | T0 | LY, | LY, | LY, | LY, | LY, | LY, | | | |
| | | SO, P | SO, P | SO, | SO, P | SO, | SO, | O, | O, | O, LP |
| | | | | LP | | P | P | LP | LP | |
| | 1 w | LY, | LY, | LY, | LY | LY, | LY, | | | |
| | | SO, a | SO, a | SO, a | SO, a | SO, | SO, | O, LP | O, LP | O, LP |
| | | few P | few P | few P | few P | a | a | | | |
| | | | | | | few P | few P | | | |
| | 2 w | LY, | LY, | LY, | LY, | LY, | LY, | | | |
| | | SO, a | SO, a | SO, a | SO, a | SO, | SO, | O, LP | O, LP | O, LP |
| | | few P | few P | few P | few P | a | a | | | |
| | | | | | | few P | few P | | | |
| | 4 w | LY, | LY, | LY, | LY, | LY, | LY, | | | |
| | | SO, a | SO, | SO, a | SO, a | SO, | SO, | O, LP | O, LP | O, LP |
| | | few P | particle | few P | few P | a | LP | | | |
| | | | free | | | few P | | | | |
| pH | T0 | 4.6 | 5.1 | 5.5 | 5.6 | 6.2 | 6.6 | 6.6 | 7.1 | 7.6 |
| | 1 w | 4.6 | 5.1 | 5.6 | 5.6 | 6.1 | 6.6 | 6.6 | 7.1 | 7.6 |
| | 2 w | 4.7 | 5.2 | 5.6 | 5.7 | 6.2 | 6.6 | 6.6 | 7.1 | 7.6 |
| | 4 w | 4.7 | 5.2 | 5.6 | 5.7 | 6.2 | 6.6 | 6.6 | 7.1 | 7.6 |
| Concentration | T0 | 50.5 | 49.1 | 49.7 | 50.6 | 50.1 | 50.5 | 50.4 | 50.0 | 51.7 |
| (mg/ml) | 1 w | 50.7 | 49.5 | 49.9 | 49.8 | 50.1 | 49.1 | 50.0 | 50.0 | 51.0 |
| | 2 w | 50.2 | 48.9 | 49.6 | 50.0 | 50.0 | 48.8 | 50.0 | 49.7 | 50.6 |
| | 4 w | 50.5 | 49.2 | 50.3 | 50.2 | 50.4 | 49.7 | 50.1 | 50.3 | 51.5 |

LY = light yellow;
SO = slightly opalescent;
O = opalescent;
P = particles;
LP = large amount of particles;
w = week(s)

### SEC-HPLC

Table 4 shows the data summary of SEC-HPLC testing results in the pH screening study.

At Time 0, % Main peak of samples in acetate or histidine buffer ranges from 97.2% to 97.9%, while % Main peak of samples in PB buffer is lower than 96.0%. After four weeks at 40° C., histidine buffer (F4-F6) showed the least monomer purity decline among all the tested buffer systems (0.7-0.8%), while PB buffer showed the most substantial monomer percentage decline (maximum 3.9%). This is illustrated in FIG. 1 as well.

High molecular weight (HMW) peak increase and low molecular weight (LMW) peak increase was observed in all nine buffers after 4 weeks at 40° C., with the minimum increase in histidine buffer and maximum increase in PB buffer.

TABLE 5
Data summary of SEC-HPLC in pH screening

| Formulation No. | | F1 | F2 | F3 | F4 | F5 | F6 | F7 | F8 | F9 |
|---|---|---|---|---|---|---|---|---|---|---|
| Main | T0 | 97.2 | 97.9 | 97.6 | 97.4 | 97.8 | 97.5 | 96.0 | 96.3 | 95.9 |
| peak | 1 w | 96.8 | 97.5 | 97.0 | 97.1 | 97.5 | 97.0 | 94.8 | 94.6 | 93.9 |
| (%) | 2 w | 96.6 | 97.3 | 96.8 | 96.9 | 97.2 | 96.9 | 94.4 | 94.3 | 93.4 |
| | 4 w | 96.2 | 97.0 | 96.5 | 96.7 | 97.1 | 96.7 | 94.0 | 93.9 | 92.0 |
| | | (1.0↓) | (0.9↓) | (1.1↓) | (0.7↓) | (0.7↓) | (0.8↓) | (2.0↓) | (2.4↓) | (3.9↓) |
| HMW | T0 | 2.8 | 2.1 | 2.4 | 2.6 | 2.2 | 2.5 | 4.0 | 3.7 | 4.1 |
| (%) | 1 w | 2.9 | 2.3 | 2.9 | 2.7 | 2.4 | 2.8 | 5.1 | 5.2 | 5.9 |
| | 2 w | 3.0 | 2.5 | 3.0 | 2.8 | 2.5 | 2.9 | 5.4 | 5.4 | 6.2 |
| | 4 w | 3.1 | 2.6 | 3.2 | 2.9 | 2.5 | 2.9 | 5.7 | 5.7 | 7.3 |
| LMW | T0 | ND | ND | ND | ND | ND | ND | ND | ND | ND |
| (%) | 1 w | 0.2 | 0.1 | 0.1 | 0.1 | 0.1 | 0.2 | 0.1 | 0.2 | 0.2 |
| | 2 w | 0.4 | 0.3 | 0.2 | 0.2 | 0.2 | 0.3 | 0.2 | 0.3 | 0.4 |
| | 4 w | 0.6 | 0.4 | 0.3 | 0.4 | 0.4 | 0.4 | 0.4 | 0.5 | 0.7 |

ND = not determined;
HMW = high molecular weight;
LMW = low molecular weight;
w = week(s);
SEC = size exclusion chromatography;
HPLC = high performance liquid chromatography

Table 6 shows the data summary of cIEF testing results in pH/buffer screening study.

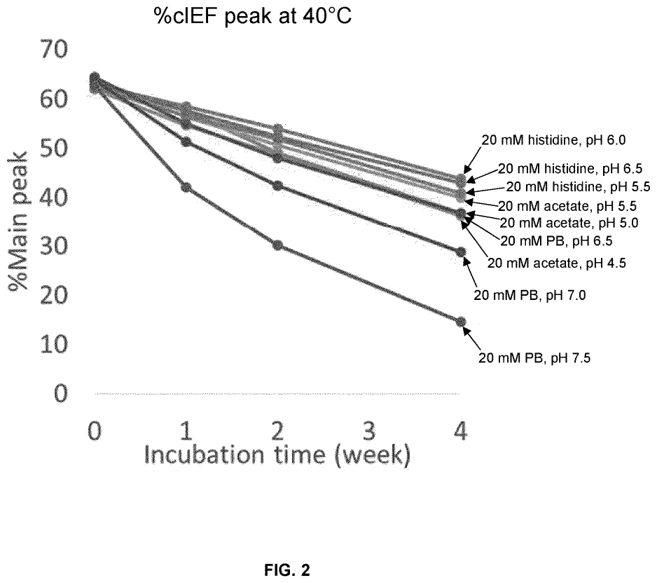

All the candidates showed main peak percentage decline, as well as acidic peak increase and basic peak decline after being stored at 40° C. for 4 weeks. Among all the candidates, there was least % Main peak decrease in histidine buffer, with a range of 19.1% to 21.6%, and maximum decrease in PB buffer, with up to 48.1% decrease in cIEF % Main peak. The trend of the cIEF % Main peak change is shown in FIG. 2.

TABLE 6
Data summary of cIEF in pH screening study

| | cIEF | | | | | | | | | | | |
|---|---|---|---|---|---|---|---|---|---|---|---|---|
| Formu- | Main peak % | | | | Acidic peak % | | | | Basic peak % | | | |
| lation | | 40° C. | | | | 40° C. | | | | 40° C. | | |
| No. | T0 | 1 w | 2 w | 4 w | T0 | 1 w | 2 w | 4 w | T0 | 1 w | 2 w | 4 w |
| F1 | 62.0 | 54.7 | 48.6 | 36.3 (25.7↓) | 29.8 | 36.9 | 43.9 | 56.3 | 8.2 | 8.4 | 7.5 | 7.4 |
| F2 | 62.7 | 57.6 | 49.1 | 36.8(25.9↓) | 28.7 | 35.1 | 44.0 | 56.8 | 8.5 | 7.3 | 6.9 | 6.4 |
| F3 | 63.4 | 56.4 | 50.6 | 40.0 (23.4↓) | 28.5 | 36.1 | 42.1 | 54.7 | 8.1 | 7.4 | 7.3 | 5.3 |
| F4 | 62.4 | 56.5 | 51.9 | 40.8 (21.6↓) | 29.6 | 35.6 | 40.6 | 53.2 | 8.0 | 7.8 | 7.5 | 6.0 |
| F5 | 62.8 | 58.5 | 54.0 | 43.7(19.1↓) | 28.8 | 34.1 | 39.2 | 50.4 | 8.4 | 7.3 | 6.8 | 5.9 |
| F6 | 63.6 | 57.6 | 52.3 | 42.9(20.7↓) | 28.6 | 35.3 | 41.6 | 51.3 | 7.8 | 7.1 | 6.1 | 5.8 |
| F7 | 64.5 | 54.9 | 47.9 | 36.8 (27.7↓) | 27.0 | 38.0 | 45.9 | 58.4 | 8.5 | 7.0 | 6.1 | 4.8 |
| F8 | 63.9 | 51.3 | 42.4 | 28.8 (35.1↓) | 27.9 | 42.0 | 52.1 | 67.7 | 8.2 | 6.7 | 5.5 | 3.5 |
| F9 | 62.6 | 42.0 | 30.1 | 14.5 (48.1↓) | 29.0 | 52.9 | 66.9 | 83.5 | 8.4 | 5.1 | 3.0 | 1.9 |

cIEF = capillary isoelectric focusing;
w = week(s)

### Caliper-SDS-(R & NR)

Table 7 summarizes the data of Caliper-SDS-(R & NR) in the pH buffer screening study. For the Caliper-SDS Reduced, all candidates showed a decrease in % heavy chain (HC)+light chain (LC), and histidine buffer candidates showed the least decrease (up to 1.2% decrease) in % HC+LC after 4 weeks of incubation at 40° C. While % HC+LC of acetate buffer candidates decreased slightly more (up to 1.7%), however, % HC+LC of PB buffer candidates decreased substantially after 4w, with a maximum of 4.7%.

A similar trend was shown in Caliper-SDS Non-Reduced results. Decrease in the % Main peak was shown in all samples after 4 weeks, and the histidine buffer showed the minimum decrease of 2.9-3.6%. The % Main peak of acetate buffer decreased 3.0% to 6.0%, while PB buffer, again, showed the most substantial change of up to 13.1% decrease in the % Main peak.

TABLE 7
Data summary of Caliper-SDS-(R & NR) in pH buffer screening study

| | Caliper-SDS Reduced | | | | Caliper-SDS Non reduced | | | |
|---|---|---|---|---|---|---|---|---|
| Formu- | % HC + LC | | | | % Main peak | | | |
| lation | | 40° C. | | | | 40° C. | | |
| No. | T0 | 1 w | 2 w | 4 w | T0 | 1 w | 2 w | 4 w |
| F1 | 99.4 | 98.8 | 98.4 | 97.7 (1.7↓) | 98.1 | 96.2 | 95.5 | 92.1 (6.0↓) |
| F2 | 99.3 | 99.0 | 98.7 | 98.1 (1.2↓) | 98.1 | 97.3 | 96.3 | 94.6 (3.5↓) |
| F3 | 99.3 | 99.1 | 98.8 | 98.3 (1.0↓) | 98.1 | 97.3 | 96.4 | 95.1 (3.0↓) |
| F4 | 99.4 | 99.0 | 98.7 | 98.2 (1.2↓) | 98.1 | 97.0 | 96.6 | 95.2 (2.9↓) |
| F5 | 99.3 | 99.0 | 98.8 | 98.3 (1.0↓) | 98.2 | 97.4 | 96.8 | 95.3 (2.9↓) |
| F6 | 99.3 | 99.0 | 98.8 | 98.2 (1.1↓) | 98.1 | 97.4 | 96.4 | 94.5 (3.6↓) |
| F7 | 99.3 | 98.8 | 98.5 | 97.6 (1.7↓) | 98.1 | 96.7 | 95.6 | 93.2 (4.9↓) |
| F8 | 99.3 | 98.6 | 98.0 | 96.7 (2.6↓) | 98.1 | 96.1 | 94.5 | 90.7 (7.4↓) |
| F9 | 99.3 | 98.0 | 96.9 | 94.6 (4.7↓) | 97.9 | 95.0 | 91.7 | 84.8 (13.1↓) |

SDS = sodium dodecyl sulfate;
R = reduced;
NR = non-reduced;
HC = heavy chain;
LC = light chain;
w = week(s)

### ELISA Binding Potency

After incubation at 40° C. for 4 weeks, a sample (20 mM histidine, pH 6.0) was sent for ELISA binding antigen assay. Results showed that the binding antigen of this sample is 105%, which indicated there was no substantial change in potency for the anti-TTR antibody after incubation at 40° C. for 4 weeks, when formulated in histidine buffer at pH 6.0.

### DLS (kD & Zeta Potential)

For all the pH buffer candidates, kD value was measured by high throughput DLS. Zeta potential of all nine samples were measured by DLS, as well as a sample with NaCl addition, to investigate whether adding NaCl could improve the stability of the antibody. The results are summarized in Table 8.

TABLE 8
Data summary of kD and zeta potential
value in pH buffer screening study

| | k_{D} | k_{D} | Zeta |
|---|---|---|---|
| | (mL/g) | (mL/g) | potential |
| | 25° C. | 40° C. | (mV) |
| 20 mM acetate pH 4.5 | 36.7 | 33.0 | 10.93 |
| 20 mM acetate pH 5.0 | 19.2 | 15.4 | 12.33 |
| 20 mM acetate pH 5.5 | 15.4 | 14.0 | 8.81 |
| 20 mM histidine pH 5.5 | 13.5 | 16.5 | 10.50 |
| 20 mM histidine pH 6.0 | 19.1 | 18.6 | 12.67 |
| 20 mM histidine 200 mM | Not tested | Not tested | 1.80 |
| NaCl pH 6.0 | | | |
| 20 mM histidine pH 6.5 | 12.7 | 15.0 | 6.66 |
| 20 mM PB pH 6.5 | −4.8 | −4.3 | 2.29 |
| 20 mM PB pH 7.0 | −7.4 | −23.1 | 0.08 |
| 20 mM PB pH 7.5 | −8.0 | −12.4 | −2.05 |

k_{D} = dissociation constant;
PB = phosphate buffer

Regarding the kD value, samples in acetate or histidine buffer showed positive value (12.7 mL/g to 36.7 mL/g at 25° C., and 14.0 mL/g to 33.0 mL/g at 40° C.), while PB buffer showed negative values. This indicated that the interaction between antibodies in acetate or histidine buffer was repulsive, thus aggregation was limited, and the antibody in phosphate buffer showed more attractive interaction, which could possibly lead to more aggregation.

Regarding the zeta potential, samples in acetate or histidine buffer showed larger values comparing with samples in phosphate buffer, indicating that the antibody was more stable in these two buffers than that in phosphate buffer.

Zeta potential value in 20 mM histidine pH 6.0 was 12.67 mV, and this number decreased to 1.80 mV when there was 200 mM NaCl added into the buffer. This suggested that the addition of NaCl damaged the colloidal stability of the anti-TTR antibody.

### Summary

Slightly opalescence particles were observed in all pH buffers. At higher pH (e.g., phosphate buffer, pH 6.5, 7.0, and 7.5) a large amount of particles and turbid solution were observed. No substantial change was observed in appearance, pH value, and antibody concentration after a 4w, 40° C. incubation.

In acetate or histidine buffer, kD value and zeta potential value was higher than that in the phosphate buffer, indicating the colloidal stability of the antibody was better in acetate or histidine buffer. The addition of NaCl into the buffer decreased the zeta potential of the antibody in 20 mM histidine buffer pH 6.0, which suggested that NaCl did not improve the colloidal stability of the protein.

Based on the SEC-HPLC results, the least % Main peak decrease among all the sample was observed in antibodies in histidine buffer (especially pH 5.5 and 6.0) after storage at 40° C. for 4 weeks.

Antibodies in acetate buffer showed slightly more % Main peak decrease, while antibodies in phosphate buffer showed maximum decrease in SEC-HPLC % Main peak.

The least decrease in cIEF % Main peak after 4 weeks incubation at 40° C. was detected for antibodies formulated in histidine buffer (pH 5.5 and 6.0), and the maximum decrease was observed for antibody in phosphate buffer.

For antibodies in acetate buffer (pH 5.0 and 5.5) after 40° C. incubation and histidine (pH 5.5, 6.0, and 6.5), a minimum decrease was observed for Caliper reduced % HC+LC, while for antibodies in acetate pH 4.5 or phosphate buffer, a substantial decrease was observed in % HC+LC. Regarding Caliper non-reduced: acetate buffer (pH 5.5) and histidine buffer (pH 5.5 and 6.0) show the least decrease in % IgG purity after 40° C. stress.

Overall, the range of histidine pH 5.5 to 6.0 was identified as providing a relatively more stable condition for the antibody, and histidine pH 5.8 buffer provided favorable stability.

### Solubility Study

The antibody was prepared to a relative high concentration (150 mg/mL) in the 20 mM histidine buffer, pH 5.8, with and without NaCl, as well as in the phosphate buffered saline (PBS), to study the solubility of the antibody. Antibody stability was studied at 2-8° C. and 25° C.

### Sample Preparation

Three formulations were used in the solubility study, the details of which are listed in Table 9. The three formulations were 20 mM histidine pH 5.8, 20 mM histidine pH 5.8 with NaCl, and PBS. The starting material (e.g., the 50 L pool DS) was buffer exchanged into these buffers and concentrated to above 150 mg/mL. All formulation samples were eventually filtered with 0.22 μm polyvinylidene difluoride (PVDF) filter, filled into 2 ml glass vials (1 ml/vial), stoppered and sealed in bio-safety hood.

TABLE 9
Formulation candidates list for solubility study

| Formulation | | | Antibody |
|---|---|---|---|
| No. | Buffer System | pH | Conc. |
| 1 | 20 mM histidine | 5.8 | 150 mg/mL |
| 2 | 20 mM histidine 155 mM NaCl | 5.8 | |
| 3 | PBS (1.1 mM NaH₂PO₄, 3.0 mM | 7.4 | |
| | Na₂HPO₄, 155 mM NaCl) | | |

PBS = phosphate buffered saline

### Study Parameters

Table 10 shows the evaluated condition for the solubility study. Samples were stored at 25° C. or 5° C. for 2 days (D). Samples were retrieved at each time point and kept at 2-8° C. before analysis. Testing items including appearance, antibody concentration, turbidity, and viscosity.

TABLE 10
Sampling and testing plan of solubility study

| | |
|---|---|
| Formulation | 48 Hours |

| No. | Time 0 | 2-8° C. | 25° C. |
|---|---|---|---|
| F1-F3 | X, Y | X | X |

X = Appearance, protein concentration, turbidity (UV350);
Y = viscosity

### Results of the Solubility Study

### Appearance and Antibody Concentration

Appearance, turbidity, and antibody concentration for solubility study are reported in Table 11. Sample in 20 mM histidine buffer pH 5.8 (F1) remained slightly yellow, slightly opalescent, and free of particles during the study. Sample in 20 mM histidine buffer pH 5.8 with 155 mM NaCl (F2) showed slightly yellow, slightly opalescent, and free of particles at time zero (T0), but became opalescent after 48 hours of incubation at either 5° C. or 25° C. Antibodies in PBS buffer (F3) showed slightly yellow, opalescent, and free of particles during the study. Large amount of air bubbles existed, and it was very difficult to remove these bubbles in these highly viscous samples.

No substantial change was observed in the turbidity or antibody concentration for all samples during the study. The variation of turbidity data may result from the existence of large amount of air bubbles and the fluctuation of antibody concentration was due to the dilution variation.

TABLE 11
Data summary of appearance, pH and antibody
concentration in buffer screening study

| Formu- | Appearance | | | Turbidity | | | Antibody conc. (mg/mL) | | |
|---|---|---|---|---|---|---|---|---|---|
| lation | | 2-8° C. | 25° C. | | 2-8° C. | 25° C. | | 2-8° C. | 25° C. |
| No. | T0 | 48 H | 48 H | T0 | 48 H | 48 H | T0 | 48 H | 48 H |
| F1 | SY, | SY, | SY, | 0.130 | 0.212 | 0.204 | 148.5 | 157.8 | 169.7 |
| | SO, | SO, | SO, | | | | | | |
| | FP | FP | FP | | | | | | |
| F2 | SY, | SY, O, | SY, O, | 0.216 | 0.142 | 0.180 | 192.5 | 189.5 | 187.8 |
| | SO, | FP | FP | | | | | | |
| | FP | | | | | | | | |
| F3 | SY, | SY, O, | SY, O, | 0.225 | 0.216 | 0.211 | 167.2 | 160.7 | 183.2 |
| | O, | FP | FP | | | | | | |
| | FP | | | | | | | | |

SY = Slightly yellow;
SO = Slightly opalescent;
O = Opalescent;
FP = free of particles;
H = hours

### Viscosity

Samples were diluted to 150 mg/mL for viscosity measurement. Viscosity data is displayed in Table 12. All samples showed shear thinning behavior and the viscosity of 20 mM histidine pH 5.8 with NaCl was the lowest (16 cP), while viscosity of 20 mM histidine pH 5.8 (F1) and PBS buffer (F3) was the highest (about 60 cP).

TABLE 12
Viscosity data for solubility study

| No | Formulation | Viscosity (cP) |
|---|---|---|
| F1 | 20 mM histidine pH 5.8 | 59.9 |
| F2 | 20 mM histidine 200 Mm NaCl PH 5.8 | 16.1 |
| F3 | PBS pH 7.4 | 59.2 |

cP = centipoise

### Summary

The solubility study investigated 3 different pH buffer types at antibody concentrations over 150 mg/mL. The results showed that no substantial change was observed in antibody concentration or turbidity at 2-8° C. and 25° C. for 48 hours, indicating that the anti-TTR antibody was stable at 150 mg/mL concentration for short term. The 20 mM histidine pH 5.8 buffer showed favorable appearance among the three investigated formulations: slightly yellow, slightly opalescent, and free of particles.

The viscosity of the samples was high at an antibody concentration of 150 mg/mL.

Based on above results, the anti-TTR antibody had favorable stability in 20 mM histidine, pH 5.8 buffer.

Excipients Screening Study: Stability at 40° C. And During Freeze-Thaw Sample Preparation Six formulations were evaluated in excipients screening, as listed in Table 13. The starting material (e.g., the 50 L pool DS) was formulated in either 20 mM histidine, pH 5.8 or 20 mM acetate acid-histidine buffer pH 5.8. Stock solution of 40% weight per unit volume (w/v) sucrose, 44.2% (w/v) trehalose 2H2O, 40% (w/v) sorbitol, 40% (w/v) I-arginine HCl, and 10% (w/v) polysorbate 80 (PS80) were prepared. The amount of DS and excipient stock solution was calculated, compounded based on the formulation recipes. All formulation samples were eventually filtered with 0.22 μm PVDF filter, filled into 2 mL glass vials (1 mL/vial), stoppered, and sealed in bio-safety hood. Antibody concentration of 100 mg/mL was used in this study.

TABLE 13
Formulation candidates list for excipients screening study

| Formu- | | | | |
|---|---|---|---|---|
| lation | | Antibody | | |
| No. | Buffer system | Conc. | Excipient | PS80 |
| F1 | 20 mM histidine pH 5.8 | 100 | 8% sucrose | 0 |
| F2 | 20 mM histidine pH 5.8 | mg/mL | 8.8% trehalose | 0 |
| | | | 2H₂O | |
| F3 | 20 mM histidine pH 5.8 | | 5% sorbitol | 0 |
| F4 | 20 mM histidine pH 5.8 | | 150 mM | 0 |
| | | | arginine-HCl | |
| F5 | 20 mM histidine-acetate | | 8% sucrose | 0 |
| | acid pH 5.8 | | | |
| F6 | 20 mM histidine pH 5.8 | | 8% sucrose, | 0.02% |
| | | | 0.02% PS80 | |

PS80 = polysorbate 80

### Study Parameters

Table 14 shows the detailed plan for excipients screening study. Samples were stored at 40° C. for 4 weeks, or freeze-thawed for 3 or 5 cycles. Samples were retrieved at each time point and kept at 2-8° C. before analysis. Testing parameters including appearance, pH, antibody concentration, osmolality, SEC-HPLC, cIEF, Caliper-SDS (reduced and non-reduced) and MFI were performed in this study.

TABLE 14
Sampling and testing plan of excipients screening study

| Formu- | | | | | | |
|---|---|---|---|---|---|---|
| lation | | Freeze thaw (−70° C. to RT) | | 40° C. | | |
| No. | T0 | 3 cycles | 5 cycles | 1 w | 2 w | 4 w |
| F1-F6 | X, Y, Z | X | X, Y | X | X | X |

Testing item: X = Appearance, pH, antibody concentration, SEC-HPLC, Caliper-SDS (non-reduced and reduced), cIEF,
Y = MFI;
Z = Osmolality,
kD (DLS);
RT = room temperature

### Results of the Excipients Screen 40° C. Stability Study

### Appearance, pH, Antibody Concentration and Osmolality

Appearance, pH, antibody concentration, and osmolality results from 40° C. stability study are presented in Table 15. For samples without PS 80 in the formulation (F1-F5), the sample was slightly yellow and slightly opalescent during the 4 week study. Samples at T0 were free of particles and 40° C. incubated samples were not free of particles.

For the sample containing PS80 in the formulation (F6), the sample remained slightly yellow, slightly opalescent and free of particles during the study, indicating that the addition of PS80 bolstered the stability of the anti-TTR antibody during storage at 40° C.

No substantial change was observed in pH value or antibody concentration in 4 weeks incubation at 40° C.

The osmolality of all samples with excipients ranged from 339 to 423 mOsm/kg.

TABLE 15
Data summary of appearance, pH, antibody concentration and
osmolality in excipients screening 40° C. stability study

| Formu- | Appearance | | | | pH | | | | Antibody conc. (mg/mL) | | | | Osmolality |
|---|---|---|---|---|---|---|---|---|---|---|---|---|---|
| lation | | 40° C. | | | | 40° C. | | | | 40° C. | | | (mOsm/kg) |
| No. | T0 | 1 w | 2 w | 4 w | T0 | 1 w | 2 w | 4 w | T0 | 1 w | 2 w | 4 w | T0 |
| F1 | SY, | SY, SO, | SY, SO, | SY, SO, | 5.9 | 6.0 | 5.9 | 5.9 | 98.9 | 104.0 | 98.7 | 97.2 | 344 |
| | SO, FP | P | P | P | | | | | | | | | |
| F2 | SY, | SY, SO, | SY, SO, | SY, SO, | 5.9 | 5.9 | 5.9 | 5.8 | 107.1 | 102.6 | 103.1 | 97.3 | 349 |
| | SO, FP | P | P | P | | | | | | | | | |
| F3 | SY, | SY, SO, | SY, SO, | SY, SO, | 5.9 | 5.9 | 5.9 | 5.8 | 106.4 | 104.3 | 103.6 | 99.7 | 423 |
| | SO, FP | P | P | P | | | | | | | | | |
| F4 | SY, O, | SY, SO, | SY, SO, | SY, O, P | 5.8 | 5.9 | 5.9 | 5.9 | 102.5 | 99.4 | 98.6 | 96.4 | 349 |
| | FP | P | P | | | | | | | | | | |
| F5 | SY, | SY, SO, | SY, SO, | SY, SO, | 5.9 | 5.9 | 5.9 | 5.8 | 104.5 | 104.4 | 104.8 | 99.5 | 388 |
| | SO, FP | P | P | P | | | | | | | | | |
| F6 | SY, | SY, SO, | SY, SO, | SY, SO, | 5.9 | 5.9 | 5.9 | 5.9 | 105.7 | 106.0 | 108.8 | 101.7 | 339 |
| | SO, FP | FP | FP | FP | | | | | | | | | |

### SEC-HPLC

Table 16 shows the data summary of SEC-HPLC testing results in excipients screening 40° C. study. When stored at 40° C. for 4 weeks, the main peak percentage of all samples, except F4, declined ˜3.3% and no substantial difference was observed among these five samples. For F4 (arginine HCl as excipient), a slightly more substantial decrease in the % Main peak can be observed (3.8%) after 4 week storage at 40° C.

TABLE 16
Data summary of SEC-HPLC in excipients screening study

| | SEC-HPLC | | | | | | | | | | | |
|---|---|---|---|---|---|---|---|---|---|---|---|---|
| | Main peak % | | | | HMW % | | | | LMW % | | | |
| Formulation | | 40° C. | | | | 40° C. | | | | 40° C. | | |
| No. | T0 | 1 w | 2 w | 4 w | T0 | 1 w | 2 w | 4 w | T0 | 1 w | 2 w | 4 w |
| F1 | 97.0 | 96.7 | 96.0 | 93.6 | 3.0 | 3.3 | 3.7 | 4.0 | ND | 0.1 | 0.3 | 2.4 |
| | | | | (3.4↓) | | | | | | | | |
| F2 | 97.3 | 97.0 | 96.4 | 94.0 | 2.8 | 2.9 | 3.3 | 3.6 | ND | 0.1 | 0.3 | 2.4 |
| | | | | (3.3↓) | | | | | | | | |
| F3 | 97.1 | 96.7 | 96.2 | 93.9 | 2.9 | 3.2 | 3.5 | 3.9 | ND | 0.1 | 0.3 | 2.3 |
| | | | | (3.2↓) | | | | | | | | |
| F4 | 97.7 | 97.5 | 96.6 | 93.9 | 2.3 | 2.4 | 3.1 | 3.4 | ND | 0.1 | 0.3 | 2.7 |
| | | | | (3.8↓) | | | | | | | | |
| F5 | 97.5 | 97.3 | 96.7 | 94.3 | 2.5 | 2.6 | 3.0 | 3.4 | ND | 0.1 | 0.3 | 2.3 |
| | | | | (3.2↓) | | | | | | | | |
| F6 | 97.4 | 97.2 | 96.4 | 94.1 | 2.6 | 2.7 | 3.2 | 3.5 | ND | 0.1 | 0.3 | 2.4 |
| | | | | (3.3↓) | | | | | | | | |

HMW = high molecular weight;
MLW = low molecular weight;
w = week(s);
SEC = size exclusion chromatography;
HPLC = high performance liquid chromatography

cIEF

Table 17 shows the summary of cIEF testing results in excipients screening 40° C. study.

When stored at 40° C. for 4 weeks, the main peak percentage of all samples declined ˜20% and the Main peak percentage of F5 showed slightly greater decline (25.8%), relative to the other formulations. Substantial increases in Acidic peak percentage and slight decreases in basic peak percentage were also be observed after 4 weeks incubation at 40° C.

TABLE 17
Data summary of cIEF in excipients screening study

| | cIEF | | | | | | | | | | | |
|---|---|---|---|---|---|---|---|---|---|---|---|---|
| Formu- | Main peak % | | | | Acidic peak % | | | | Basic peak % | | | |
| lation | | 40° C. | | | | 40° C. | | | | 40° C. | | |
| No. | T0 | 1 w | 2 w | 4 w | T0 | 1 w | 2 w | 4 w | T0 | 1 w | 2 w | 4 w |
| F1 | 62.6 | 55.7 | 53.9 | 42.3 | 29.6 | 36.5 | 39.5 | 51.2 | 7.8 | 7.8 | 6.6 | 6.5 |
| | | | | (20.3↓) | | | | | | | | |
| F2 | 62.0 | 56.8 | 53.0 | 42.9 | 29.6 | 35.7 | 39.9 | 50.4 | 8.3 | 7.5 | 7.1 | 6.7 |
| | | | | (19.1↓) | | | | | | | | |
| F3 | 63.0 | 55.3 | 51.9 | 42.1 | 28.9 | 36.6 | 41.1 | 52.0 | 8.1 | 8.1 | 6.9 | 5.9 |
| | | | | (20.9↓) | | | | | | | | |
| F4 | 62.0 | 57.1 | 52.8 | 44.5 | 30.5 | 34.7 | 39.5 | 48.6 | 7.5 | 8.2 | 7.7 | 6.9 |
| | | | | (17.5↓) | | | | | | | | |
| F5 | 63.8 | 55.0 | 49.2 | 38.0 | 28.2 | 37.5 | 44.2 | 56.2 | 8.0 | 7.5 | 6.6 | 5.9 |
| | | | | (25.8↓) | | | | | | | | |
| F6 | 61.6 | 55.4 | 52.1 | 41.4 | 30.7 | 36.9 | 40.8 | 52.4 | 7.8 | 7.6 | 7.0 | 6.2 |
| | | | | (20.2↓) | | | | | | | | |

### Non Reduced Caliper-SDS

Table 18 shows the data summary of Caliper-SDS non-reduced testing results in the excipients screening 4000 study. At time zero (T0), the purity of all formulations was 97.1% and there was ˜3.5% decrease after four weeks storage at 4000. No substantial difference was observed among six formulations.

TABLE 18
Data summary of Caliper-SDS non-reduced
in excipients screening study

Purity %

| Formulation | | 40° C. | | |
|---|---|---|---|---|
| No. | T0 | 1 w | 2 w | 4 w |
| F1 | 97.1 | 96.2 | 95.6 | 93.7 (3.4↓) |
| F2 | 97.1 | 96.2 | 95.3 | 93.6 (3.5↓) |
| F3 | 97.1 | 96.3 | 95.4 | 93.9 (3.2↓) |
| F4 | 97.1 | 96.2 | 95.3 | 93.3 (3.8↓) |
| F5 | 97.1 | 96.2 | 95.4 | 93.5 (3.6↓) |
| F6 | 97.1 | 96.2 | 95.4 | 93.6 (3.5↓) |

### Reduced Caliper-SDS

Table 19 shows the data summary of reduced Caliper-SDS results in excipients screening 40° C. study. At time zero (T0), the purity of all formulation was ˜99.0% and there was a ˜1.6% decrease after four weeks of storage at 40° C. No substantial difference was observed among six formulations.

TABLE 19
Data summary of Reduced Caliper-
SDS in excipients screening study

HC + LC %

| Formulation | | 40° C. | | |
|---|---|---|---|---|
| No. | T0 | 1 w | 2 w | 4 w |
| F1 | 99.1 | 98.5 | 98.2 | 97.4 (1.7↓) |
| F2 | 98.9 | 98.7 | 98.3 | 97.3 (1.6↓) |
| F3 | 98.9 | 98.6 | 98.2 | 97.4 (1.5↓) |
| F4 | 98.9 | 98.5 | 98.2 | 97.4 (1.5↓) |
| F5 | 98.9 | 98.6 | 98.2 | 97.4 (1.5↓) |
| F6 | 98.9 | 98.5 | 98.2 | 97.3 (1.6↓) |

HC = heavy chain;
LC = light chain;
w = week(s)

### Summary of Excipients Screen, 40° C. Stability Study

Large amount of air bubbles and particles were observed in samples without PS80. Samples containing 0.02% PS80 were free of visible particles, suggesting that PS80 imparts stability to the composition by reducing aggregation of anti-TTR antibodies. All samples were observed as slightly yellow and slightly opalescence, and no substantial change was observed in each formulation after 4 weeks incubation at 40° C.

No substantial change was observed in pH or concentration after 4 weeks incubation at 40° C. The osmolality for all six samples were higher than isotonic.

A decrease was observed in SEC purity, cIEF main peak, and Caliper R and NR purity for all six formulations, with F4 showing the biggest decrease in SEC purity, and F5 showing the highest decrease in cIEF Main peak percentage.

No substantial difference can be observed between F1 and F6, which indicated that the addition of PS80 improved the appearance of the sample without reducing purity of SEC, cIEF, and Caliper R and NR.

To sum up, formulations containing sucrose or sorbitol performed with favorable results among all the formulations based on 40° C. stability study. Furthermore, the addition of PS80 to the 8% sucrose formulation (F6) resulted in no visible particles after incubation at 40° C., indicating that the addition of PS80 bolstered the stability of the anti-TTR antibody during storage at 40° C.

### Results of the Excipient Study: Freeze Thaw Study

The stability of all six formulations under three and five cycles freeze-thaw was evaluated in this excipients study. The results are described below.

### Appearance, pH, Antibody Concentration and Osmolality

Appearance, pH, and antibody concentration in this freeze-thaw study is presented in Table 20. For samples without PS 80 in the formulation (F1-F5), samples showed slightly yellow and slightly opalescent after the 3/5 cycles of freeze-thaw study. Samples at T0 were free of particles and freeze-thawed samples had particles.

For the sample with PS80 in the formulation (F6), sample remained slightly yellow, slightly opalescent and free of particles during the study, indicating that the addition of PS80 bolstered the stability of the anti-TTR antibody during freeze-thaw cycles.

No substantial change was observed in the pH value or antibody concentration after up to 5 cycles of freeze-thawing.

TABLE 20
appearance, pH and antibody concentration in freeze-thaw study of excipients screening

| | | | | pH | | | Antibody conc. (mg/mL) | | |
|---|---|---|---|---|---|---|---|---|---|
| Formu- | Appearance | | | | Freeze thaw | | | Freeze thaw | |
| lation | | Freeze thaw | | | 3 | 5 | | 3 | 5 |
| No. | T0 | 3 cycles | 5 cycles | T0 | cycles | cycles | T0 | cycles | cycles |
| F1 | SY, SO, | SY, SO, P | SY, SO, P | 5.9 | 6.0 | 6.0 | 98.9 | 102.3 | 102.5 |
| | FP | | | | | | | | |
| F2 | SY, SO, | SY, SO, P | SY, SO, P | 5.9 | 5.9 | 6.0 | 107.1 | 101.9 | 101.2 |
| | FP | | | | | | | | |
| F3 | SY, | SY, SO, P | SY, SO, P | 5.9 | 5.9 | 5.9 | 106.4 | 104.0 | 103.6 |
| | SO, | | | | | | | | |
| | FP | | | | | | | | |
| F4 | SY, O, FP | SY, SO, P | SY, SO, P | 5.8 | 5.9 | 5.9 | 102.5 | 98.0 | 98.5 |
| F5 | SY, SO, | SY, SO, P | SY, SO, P | 5.9 | 5.9 | 5.9 | 104.5 | 100.9 | 100.5 |
| | FP | | | | | | | | |
| F6 | SY, SO, | SY, SO, | SY, SO, | 5.9 | 5.9 | 5.9 | 105.7 | 102.5 | 102.3 |
| | FP | FP | FP | | | | | | |

SY: slightly yellow,
SO: slightly opalescence,
P: particles,
FP: free of particles

### SEC-HPLC

Table 21 shows the summary of SEC-HPLC testing results in the excipients screening freeze-thaw study. After ≤5 cycles of freeze-thaw, no substantial change was observed in SEC-HPLC Main.—For F4 (arginine HCl as excipient), a slight decrease in % Main peak can be observed (0.9%) after 5 cycles of freeze-thaw.

TABLE 21
Data summary of SEC-HPLC in excipients screening study

| | SEC-HPLC | | | | | | | | |
|---|---|---|---|---|---|---|---|---|---|
| | Main peak % | | | HMW % | | | LMW % | | |
| | | Freeze thaw | | | Freeze thaw | | | Freeze thaw | |
| Formulation | | 3 | 5 | | 3 | 5 | | 3 | 5 |
| No. | T0 | cycles | cycles | T0 | cycles | cycles | T0 | cycles | cycles |
| F1 | 97.0 | 96.8 | 96.7 | 3.0 | 3.2 | 3.3 | ND | ND | ND |
| F2 | 97.3 | 97.1 | 97.1 | 2.8 | 2.9 | 2.9 | ND | ND | ND |
| F3 | 97.1 | 97.0 | 97.0 | 2.9 | 3.0 | 3.0 | ND | ND | ND |
| F4 | 97.7 | 97.0 | 96.8 (0.9↓) | 2.3 | 3.0 | 3.2 | ND | ND | ND |
| F5 | 97.5 | 97.4 | 97.3 | 2.5 | 2.6 | 2.7 | ND | ND | ND |
| F6 | 97.4 | 97.3 | 97.2 | 2.6 | 2.7 | 2.8 | ND | ND | ND |

cIEF

Table 22 shows the summary of cIEF testing results in excipients screening study.

After 5 cycles of freeze-thaw, a 2.5% or 1.5% decrease was observed in cIEF main peak percentage in Formulation 3 (sorbitol as excipients) and Formulation 5 (acetate acid-histidine buffer, sucrose as excipients) respectively. No substantial change was observed in other formulation after up to five cycles of freeze-thaw.

TABLE 22
Data summary of cIEF5 in excipients screening study

| | cIEF | | | | | | | | |
|---|---|---|---|---|---|---|---|---|---|
| | Main peak % | | | Acidic peak % | | | Basic peak % | | |
| Formulation | | Freeze thaw | | | Freeze thaw | | | Freeze thaw | |
| No. | T0 | 3 cycles | 5 cycles | T0 | 3 cycles | 5 cycles | T0 | 3 cycles | 5 cycles |
| F1 | 62.6 | 61.2 | 62.9 | 29.6 | 29.9 | 28.6 | 7.8 | 8.8 | 8.6 |
| F2 | 62.0 | 61.3 | 61.7 | 29.6 | 30.4 | 30.0 | 8.3 | 8.4 | 8.3 |
| F3 | 63.0 | 61.2 | 60.5 | 28.9 | 30.2 | 31.3 | 8.1 | 8.6 | 8.2 |
| | | | (2.5↓) | | | | | | |
| F4 | 62.0 | 63.4 | 61.9 | 30.5 | 28.3 | 29.6 | 7.5 | 8.3 | 8.5 |
| F5 | 63.8 | 61.4 | 62.3 | 28.2 | 30.4 | 29.6 | 8.0 | 8.2 | 8.1 |
| | | | (1.5↓) | | | | | | |
| F6 | 61.6 | 63.0 | 62.0 | 30.7 | 28.8 | 29.9 | 7.8 | 8.2 | 8.1 |

### Non Reduced Caliper-SDS

Table 23 shows the data summary of non-reduced Caliper-SDS testing results in the excipients screening freeze-thaw study. At time zero (T0), the purity of all formulation was 97.1% and there was no substantial change observed in purity after 5 cycles of freeze-thaw.

TABLE 23
Data summary of non-reduced Caliper-
SDS in excipients screening study

Purity %

| | |
|---|---|
| Formulation | Freeze thaw |

| No. | T0 | 3 cycles | 5 cycles |
|---|---|---|---|
| F1 | 97.1 | 97.1 | 97.1 |
| F2 | 97.1 | 97.3 | 97.2 |
| F3 | 97.1 | 97.2 | 97.2 |
| F4 | 97.1 | 97.0 | 97.1 |
| F5 | 97.1 | 97.1 | 97.2 |
| F6 | 97.1 | 97.0 | 97.1 |

### Reduced Caliper-SDS

Table 24 shows the data summary of reduced Caliper-SDS testing results in the excipients screening study. At time zero (T0), the purity of all formulation was ˜99.0% and there was no substantial decrease after 5 cycles of freeze-thaw.

TABLE 24
Data summary of Reduced Caliper-SDS in
excipients screening freeze-thaw study

HC + LC %

| | |
|---|---|
| Formulation | Freeze-thaw |

| No. | T0 | 3 cycles | 5 cycles |
|---|---|---|---|
| F1 | 99.1 | 98.9 | 98.8 |
| F2 | 98.9 | 98.9 | 98.8 |
| F3 | 98.9 | 98.9 | 98.9 |
| F4 | 98.9 | 99.0 | 99.0 |
| F5 | 98.9 | 98.8 | 98.9 |
| F6 | 98.9 | 98.8 | 98.8 |

HC = heavy chain;
LC = light chain

### Subvisible Particles (MFI)

Subvisible particles measured by MFI in excipients screen study is shown in Table 25. For formulation without PS80 (F1-F5), a large number of subvisible particles were detected starting from time zero (T0), while for the sample with PS80 (F6), the number was much lower. The addition of PS80 substantially decreased the number of subvisible particles in the samples. No substantial change was observed for all formulations before and after 5 cycles of freeze-thaw.

TABLE 25
Subvisible particles measured by MFI in
excipients screening freeze-thaw study

| | |
|---|---|
| Formu- | MFI (number/mL) AR < 0.85 |

| lation | | ECD ≥ 2 | ECD ≥ 10 | ECD ≥ 25 |
|---|---|---|---|---|
| No | | μm | μm | μm |
| 1 | T0 | 6436 | 409 | 22 |
| | 5C | 99029 | 2375 | 25 |
| 2 | T0 | 97867 | 2720 | 78 |
| | 5C | 49282 | 1552 | 28 |
| 3 | T0 | 114554 | 3353 | 64 |
| | 5C | 38065 | 1526 | 35 |
| 4 | T0 | 187782 | 6670 | 479 |
| | 5C | 69745 | 5210 | 215 |
| 5 | T0 | 13077 | 705 | 48 |
| | 5C | 43810 | 1151 | 27 |
| 6 | T0 | 7001 | 289 | 27 |
| | 5C | 3676 | 92 | 0 |

MFI = microfluidic imaging;
ECD = Equivalent Circular Diameter;
AR = aspect ratio

### Freeze-Thaw Study Summary

Large amount of air bubbles and particles were difficult to differentiate in samples without PS80, while samples containing 0.02% PS80 were free of visible particles, suggesting that PS80 imparts stability to the composition by reducing aggregation of anti-TTR antibodies. All samples were observed as slightly yellow and slightly opalescence, and no substantial change was observed for each formulation after 5 cycles of freeze-thaw. A large number of subvisible particles was detected in formulations without PS80, while a substantial decrease of subvisible particles was observed when PS80 was added into the formulation.

No substantial change was observed in pH or concentration after 5 cycles of freeze-thaw.

For F4 (arginine HCl as excipient), a 0.9% decrease was observed in SEC Main peak percentage, while a substantial change was observed in SEC Main peak percentage for other five formulations. Regarding the cIEF, F3 (sorbitol as excipients) or F5 (acetate acid-histidine as buffer) showed slightly decrease in Main peak percentage, while for other formulations, no substantial change was observed in cIEF % Main peak after 5 cycles of freeze-thaw. Comparing all the excipients, formulations with sucrose had the best antibody stability in 20 mM histidine buffer.

No substantial change was observed in Caliper NR and R for all samples after 5 cycles of freeze-thaw.

No substantial difference was observed between F1 and F6, which indicated the addition of PS80 improved the appearance and subvisible particles of the samples without reducing purity of SEC, cIEF, and Caliper R and NR.

To sum up, formulation containing sucrose and PS80 performed the best among all the formulations. For example, the addition of PS80 to the 8% sucrose formulation (F6) resulted in no visible particles after one or more freeze-thaw cycles, indicating that the addition of PS80 bolstered the stability of the anti-TTR antibody during storage at 40° C.

### Summary of Excipient Screening Study

To sum up the 40° C. stability and freeze-thaw study, it was observed that formulations with sucrose stabilized the antibody the most, indicated as the lowest decrease in SEC, cIEF and Caliper purity compared with other formulations. The addition of PS80 did improve the appearance and reduce the subvisible particles substantially without any negative effects to antibody stability.

20 mM histidine buffer, 8% or 6.5% (w/v) sucrose, pH 5.8 was chosen as the final buffer and excipient system. The surfactant (PS80) concentration would be evaluated in the next study.

### Surfactant Screening Study

The aim of this study was to evaluate the effect of PS80 concentration on antibody stability, and to evaluate the effect of sucrose concentration on antibody stability under different stress conditions (25° C., agitation, 40° C., and freeze-thaw).

### Sample Preparation

Six formulations were evaluated in surfactant screening as listed in Table 26. The starting material (e.g., the 50 L pool DS) was buffer exchanged into 20 mM histidine, pH 5.8. Stock solution of 60% (w/v) sucrose, 10% (w/w) PS80 were prepared. The amounts of DS, excipients stock solution and surfactant stock solution were calculated, weighed, and mixed based on the formulation recipes. All formulation samples were eventually filtered with 0.22 μm PVDF filter, filled into glass vials (2 mL/vial), stoppered and sealed in bio-safety hood. Note, 2R vials were used for all formulations except for freeze-thaw samples, which were filled into 6R samples, as 2 mL in 2R bottle resulted in vial breakage during freeze-thaw.

TABLE 26
Formulation candidates list for surfactant screening study

| Formulation | Buffer system, pH, | | |
|---|---|---|---|
| No. | Antibody Conc. | Excipient | Surfactant |
| F1 | 20 mM | 8% | 0.02% |
| | histidine, | sucrose | PS80 |
| F2 | pH 5.8, | | 0.04% |
| | 100 mg/mL | | PS80 |
| F3 | | | 0.06% |
| | | | PS80 |
| F4 | | 6.5% | 0.02% |
| | | sucrose | PS80 |
| F5 | | | 0.04% |
| | | | PS80 |
| F6 | | | 0.06% |
| | | | PS80 |

PS80 = polysorbate 80

### Study Parameters

Table 27 shows the sampling and testing plan for surfactant screening study. Samples were agitated at 25° C. with speed of 200 rpm for 3 or 7 days (D), or no agitation at 25° C. for 3 or 7 days was also evaluated. For the freeze-thaw study, samples were completely frozen at −70° C. and completely thawed at room temperature for each cycle, and 3 or 5 cycles were evaluated. Samples were also kept at 40° C. for up to 2 weeks. Samples were timely retrieved and kept at 2-8° C. before analysis. Testing items including appearance, pH, antibody concentration, osmolality, SEC-HPLC, Caliper-SDS (reduced and non-reduced), cIEF and subvisible particles (HIAC) were performed for this study.

TABLE 27
Sampling and testing plan of surfactant screening study

| | | Agitation | | No | | Freeze-Thaw | | | |
|---|---|---|---|---|---|---|---|---|---|
| Storage | | (200 rpm, | | agitation | | (−70° C./RT) | | 40° C. | |
| condi- | | 25° C.) | | (25° C.) | | 3 | 5 | 1 | 2 |
| tion | T0 | 3 D | 7 D | 3 D | 7 D | Cycles | Cycles | w | w |
| F1-F6 | X, Y | X | X | X | X | X | X | X | X |

X = Appearance, pH, antibody concentration, SEC-HPLC, non-reduced and reduced Caliper-SDS, cIEF, HIAC;
Y = Osmolality;
D = day(s)

### Results

### Appearance, pH, Osmolality and Antibody Concentration

Table 28 shows the data summary of appearance, pH, osmolality and antibody concentration for this study. After 7 days agitation at 200 rpm, all samples remained slightly yellow, slightly opalescent and free of particles.

The pH and antibody concentration of F1-F6 remained stable during the study, with pH value at 5.8-5.9, and antibody concentration at ˜100 mg/mL.

The osmolality of F1-F3 (8% sucrose) was around 340, while the osmolality of F4-F6 (6.5% sucrose) was around 280. Accordingly, F1-F3 as well as F4-F6 meet the Ph. Eur. limit of >240 mOsm/kg.

TABLE 28
Data summary of appearance, pH, osmolality and antibody
concentration in surfactant screen agitation study

| Formulation No. | | F1 | F2 | F3 | F4 | F5 | F6 |
|---|---|---|---|---|---|---|---|
| Appearance | T0 | SY, SO, | SY, SO, | SY, SO, | SY, SO, | SY, SO, | SY, SO, |
| | | free of | free of | free of | free of | free of | free of |
| | | particles | particles | particles | particles | particles | particles |
| | A-3 D | SY, SO, | SY, SO, | SY, SO, | SY, SO, | SY, SO, | SY, SO, |
| | | free of | free of | free of | free of | free of | free of |
| | | particles | particles | particles | particles | particles | particles |
| | A-7 D | SY, SO, | SY, SO, | SY, SO, | SY, SO, | SY, SO, | SY, SO, |
| | | free of | free of | free of | free of | free of | free of |
| | | particles | particles | particles | particles | particles | particles |
| pH | T0 | 5.9 | 5.8 | 5.8 | 5.8 | 5.8 | 5.8 |
| | A-3 D | 5.9 | 5.9 | 5.9 | 5.9 | 5.8 | 5.8 |
| | A-7 D | 5.8 | 5.9 | 5.9 | 5.8 | 5.8 | 5.8 |
| Antibody | T0 | 104.5 | 105.0 | 103.3 | 100.6 | 102.5 | 99.0 |
| conc. | A-3 D | 99.4 | 111.1 | 99.0 | 99.7 | 98.8 | 100.0 |
| (mg/mL) | A-7 D | 102.6 | 99.6 | 102 | 104.2 | 107.6 | 104.1 |
| Osmolarity | T0 | 343 | 339 | 336 | 279 | 285 | 281 |

SY: slightly yellow,
SO: slightly opalescent;
D = day(s)

### SEC-HPLC

SEC-HPLC results of agitation study in surfactant screening are reported in Table 29. After agitation for 7 days, the Main peak percentage of all testing samples remained stable, and no substantial difference was observed among these six samples.

TABLE 29
Data summary of SEC-HPLC in agitation study of surfactant screening

| | SEC-HPLC | | | | | | | | |
|---|---|---|---|---|---|---|---|---|---|
| Formulation | Main peak (%) | | | HMW (%) | | | LMW (%) | | |
| No. | T0 | A-3 D | A-7 D | T0 | A-3 D | A-7 D | T0 | A-3 D | A-7 D |
| F1 | 97.0 | 97.1 | 96.9 | 2.9 | 2.9 | 3.0 | 0.1 | ND | 0.1 |
| F2 | 97.1 | 97.1 | 97.0 | 2.8 | 2.9 | 2.9 | 0.1 | ND | 0.1 |
| F3 | 97.1 | 97.1 | 97.0 | 2.8 | 2.9 | 2.9 | 0.1 | ND | 0.1 |
| F4 | 97.0 | 97.1 | 96.9 | 2.9 | 2.9 | 3.0 | 0.1 | ND | 0.1 |
| F5 | 97.1 | 97.2 | 97.0 | 2.8 | 2.8 | 2.9 | 0.1 | ND | 0.1 |
| F6 | 97.1 | 97.1 | 97.0 | 2.8 | 2.9 | 2.9 | 0.1 | ND | 0.1 |

D = day(s);
HMW = high molecular weight;
LMW = low molecular weight

cIEF

The cIEF results of surfactant screening agitation study are reported in Table 30. No substantial change was observed in cIEF Main peak percentage in all six formulations after 7 days agitation at 200 rpm.

TABLE 30
Data summary of cIEF in surfactant screening agitation study

| | cIEF | | | | | | | | |
|---|---|---|---|---|---|---|---|---|---|
| Formulation | Main peak (%) | | | Acidic peak (%) | | | Basic peak (%) | | |
| No. | T0 | A-3 D | A-7 D | T0 | A-3 D | A-7 D | T0 | A-3 D | A-7 D |
| F1 | 61.2 | 59.5 | 58.7 | 30.9 | 32.5 | 33.6 | 7.8 | 8.0 | 7.7 |
| F2 | 59.9 | 59.0 | 59.6 | 31.6 | 33.0 | 32.8 | 8.5 | 7.9 | 7.6 |
| F3 | 60.9 | 59.4 | 60.2 | 30.9 | 32.5 | 31.6 | 8.2 | 8.1 | 8.2 |
| F4 | 60.9 | 59.7 | 60.0 | 30.8 | 32.6 | 32.4 | 8.2 | 7.7 | 7.7 |
| F5 | 60.7 | 59.1 | 59.4 | 31.0 | 33.3 | 32.3 | 8.3 | 7.5 | 8.3 |
| F6 | 60.3 | 59.0 | 58.4 | 31.0 | 33.4 | 33.1 | 8.7 | 7.6 | 8.5 |

### Non Reduced Caliper-SDS

Non-reduced Caliper-SDS results are reported in Table 31. The non-reduced Caliper-SDS purity percentage of all testing samples remained stable and no substantial difference was observed among these samples during the study.

TABLE 31
Data summary of non-reduced Caliper-SDS
in surfactant screening agitation study

| | |
|---|---|
| Formulation | Purity % Caliper-SDS (non-reduced) |

| No. | T0 | A-3 D | A-7 D |
|---|---|---|---|
| F1 | 97.7 | 97.7 | 97.1 |
| F2 | 97.8 | 97.8 | 97.6 |
| F3 | 97.7 | 97.6 | 97.7 |
| F4 | 97.8 | 97.6 | 96.6 |
| F5 | 97.7 | 97.7 | 97.7 |
| F6 | 97.9 | 97.7 | 97.2 |

D = day(s)

### Reduced Caliper-SDS

Reduced Caliper-SDS results of surfactant screen agitation study are reported in Table 32. The reduced Caliper SDS purity percentage of all testing samples remained stable and no substantial difference was observed among these samples during the study.

TABLE 32
Data summary of reduced Caliper-SDS in
surfactant screening agitation study

| | |
|---|---|
| Formulation | HC + LC % Caliper-SDS (Reduced) |

| No. | T0 | A-3 D | A-7 D |
|---|---|---|---|
| F1 | 99.2 | 99.2 | 99.1 |
| F2 | 99.2 | 99.2 | 99.1 |
| F3 | 99.2 | 99.2 | 99.0 |
| F4 | 99.3 | 99.2 | 99.2 |
| F5 | 99.3 | 99.1 | 99.1 |
| F6 | 99.3 | 99.1 | 99.2 |

HC = heavy chain;
LC = light chain;
D = days(s)

Sub visible particle (HIAC)

Table 33 shows the data summary of subvisible particle (HIAC) testing result for agitation study in surfactant screening. No substantial change was observed in subvisible particles in six formulation after 7 days of agitation.

TABLE 33
Data summary of subvisible particle (HIAC) in agitation study

| | HIAC | | | | | | | | |
|---|---|---|---|---|---|---|---|---|---|
| | (particles/ml) | | | | | | | | |
| Formulation | ≥2 μm | | | ≥10 μm | | | ≥25 μm | | |
| No. | T0 | A-3 D | A-7 D | T0 | A-3 D | A-7 D | T0 | A-3 D | A-7 D |
| F1 | 234 | 123 | 312 | 4 | 5 | 13 | 0 | 1 | 1 |
| F2 | 196 | 113 | 109 | 8 | 2 | 1 | 0 | 1 | 0 |
| F3 | 74 | 792 | 117 | 5 | 23 | 1 | 0 | 0 | 1 |
| F4 | 102 | 232 | 198 | 4 | 5 | 12 | 1 | 0 | 3 |
| F5 | 98 | 1743 | 3410 | 0 | 27 | 96 | 0 | 1 | 3 |
| F6 | 428 | 173 | 101 | 7 | 3 | 1 | 0 | 1 | 0 |

D = day(s)

### Summary of Agitation Study

Agitation up to 7 days was used in surfactant screening study to evaluate the stability of the formulations. No substantial difference was observed among these six different formulations after stress testing.

### 25° C. Stability

### Appearance, pH, and Antibody Concentration

Table 34 shows the data summary of appearance, pH, and antibody concentration for this study. All samples were slightly yellow, slightly opalescent, and free of particles, and no substantial change was observed after 7 days incubation at 25° C.

The pH and antibody concentration of F1-F6 remained stable during the study, with pH value at 5.8-5.9, and antibody concentration at ˜100 mg/mL.

TABLE 34
Data summary of appearance, pH and antibody in surfactant screen 25° C. study

| | | | | | | | Antibody conc. | | |
|---|---|---|---|---|---|---|---|---|---|
| Formulation | Appearance | | | pH | | | (mg/mL) | | |
| No. | T0 | 3 D | 7 D | T0 | 3 D | 7 D | T0 | 3 D | 7 D |
| F1 | SY, SO, | SY, SO, free | SY, SO, | 5.9 | 5.9 | 5.8 | 104.5 | 104.1 | 104.2 |
| | free of | of particles | free of | | | | | | |
| | particles | | particles | | | | | | |
| F2 | SY, SO, | SY, SO, free | SY, SO, | 5.8 | 5.9 | 5.9 | 105.0 | 98.7 | 102.0 |
| | free of | of particles | free of | | | | | | |
| | particles | | particles | | | | | | |
| F3 | SY, SO, | SY, SO, free | SY, SO, | 5.8 | 5.9 | 5.9 | 103.3 | 97.9 | 103.1 |
| | free of | of particles | free of | | | | | | |
| | particles | | particles | | | | | | |
| F4 | SY, SO, | SY, SO, free | SY, SO, | 5.8 | 5.8 | 5.9 | 100.6 | 100.4 | 99.6 |
| | free of | of particles | free of | | | | | | |
| | particles | | particles | | | | | | |
| F5 | SY, SO, | SY, SO, free | SY, SO, | 5.8 | 5.8 | 5.8 | 102.5 | 100.7 | 112.6 |
| | free of | of particles | free of | | | | | | |
| | particles | | particles | | | | | | |
| F6 | SY, SO, | SY, SO, free | SY, SO, | 5.8 | 5.8 | 5.8 | 99.0 | 100.5 | 110.0 |
| | free of | of particles | free of | | | | | | |
| | particles | | particles | | | | | | |

D = day(s)

### SEC-HPLC

SEC-HPLC results of surfactant screening 25° C. study are reported in Table 35. After incubation for 7 days at 25° C., the Main peak percentage of all testing samples remained stable, and no substantial difference was observed among these samples.

TABLE 35
Data summary of SEC-HPLC in 25°
C. study of surfactant screening

| | SEC-HPLC | | | | | | | | |
|---|---|---|---|---|---|---|---|---|---|
| Formulation | Main peak (%) | | | HMW (%) | | | LMW (%) | | |
| No. | T0 | 3 D | 7 D | T0 | 3 D | 7 D | T0 | 3 D | 7 D |
| F1 | 97.0 | 97.1 | 97.0 | 2.9 | 2.9 | 2.9 | 0.1 | ND | 0.1 |
| F2 | 97.1 | 97.1 | 97.0 | 2.8 | 2.9 | 2.9 | 0.1 | ND | 0.1 |
| F3 | 97.1 | 97.1 | 97.0 | 2.8 | 2.9 | 2.8 | 0.1 | ND | 0.1 |
| F4 | 97.0 | 97.0 | 97.0 | 2.9 | 3.0 | 2.9 | 0.1 | ND | 0.1 |
| F5 | 97.1 | 97.1 | 97.0 | 2.8 | 2.9 | 3.0 | 0.1 | ND | 0.1 |
| F6 | 97.1 | 97.1 | 96.9 | 2.8 | 2.9 | 3.0 | 0.1 | ND | 0.1 |

HMW = high molecular weight;
LMW = low molecular weight;
w = week(s);
SEC = size exclusion chromatography;
HPLC = high performance liquid chromatography;
D = day(s)

cIEF

The cIEF results of surfactant screening 2500 study are reported in the Table 36. No substantial change was observed in cIEF Main peak percentage for all six formulations after 7 days incubation at 2500.

TABLE 36
Data summary of cIEF in 25° C. study of surfactant screening

| Formu- | cIEF | | | | | | | | |
|---|---|---|---|---|---|---|---|---|---|
| lation | Main peak (%) | | | Acidic peak (%) | | | Basic peak (%) | | |
| No. | T0 | 3 D | 7 D | T0 | 3 D | 7 D | T0 | 3 D | 7 D |
| F1 | 61.2 | 58.0 | 58.9 | 30.9 | 34.0 | 33.4 | 7.8 | 7.9 | 7.8 |
| F2 | 59.9 | 61.1 | 59.2 | 31.6 | 31.5 | 32.5 | 8.5 | 7.5 | 8.4 |
| F3 | 60.9 | 60.4 | 58.9 | 30.9 | 31.5 | 32.4 | 8.2 | 8.2 | 8.7 |
| F4 | 60.9 | 61.2 | 59.7 | 30.8 | 31.0 | 31.7 | 8.2 | 7.8 | 8.6 |
| F5 | 60.7 | 59.8 | 59.4 | 31.0 | 32.2 | 32.6 | 8.3 | 8.0 | 8.0 |
| F6 | 60.3 | 60.2 | 58.7 | 31.0 | 31.5 | 33.3 | 8.7 | 8.3 | 8.0 |

cIEF = capillary isoelectric focusing;
D = days(s)

### Non-Reduced Caliper-SDS

Non-reduced Caliper-SDS results of surfactant screening 25° C. study are reported in Table 37. The non-reduced Caliper-SDS purity percentage of all testing samples remained stable, and no substantial difference was observed among these samples during the study.

TABLE 37
Data summary of non-reduced Caliper-
SDS in surfactant screening study

| | |
|---|---|
| Formulation | Purity % Caliper-SDS (non-reduced) |

| No. | T0 | 3 D | 7 D |
|---|---|---|---|
| F1 | 97.7 | 97.8 | 97.4 |
| F2 | 97.8 | 97.6 | 97.6 |
| F3 | 97.7 | 97.7 | 97.7 |
| F4 | 97.8 | 96.1 | 97.5 |
| F5 | 97.7 | 97.5 | 97.4 |
| F6 | 97.9 | 97.5 | 97.6 |

SDS = sodium dodecyl sulfate;
D = day(s)

### Reduced Caliper-SDS

Reduced Caliper-SDS results are reported in Table 38. The reduced Caliper SDS purity percentage of all testing samples remained stable, and no substantial difference was observed among these samples during the study.

TABLE 38
Data summary of reduced Caliper-
SDS in surfactant screening study

| | |
|---|---|
| Formulation | HC + LC % Caliper-SDS (Reduced) |

| No. | T0 | 3 D | 7 D |
|---|---|---|---|
| F1 | 99.2 | 99.0 | 99.1 |
| F2 | 99.2 | 99.2 | 99.2 |
| F3 | 99.2 | 99.2 | 99.1 |
| F4 | 99.3 | 99.1 | 99.2 |
| F5 | 99.3 | 99.1 | 99.2 |
| F6 | 99.3 | 99.0 | 99.0 |

SDS = sodium dodecyl sulfate;
D = day(s);
HC = heavy chain;
LC = light chain

### Subvisible Particles (HIAC)

Table 39 shows the data summary of subvisible particle (HIAC) testing result for 25° C. study in surfactant screening. No substantial change was observed in subvisible particles in the six formulations after 7 days incubation at 25° C.

TABLE 39
Data summary of subvisible particle (HIAC) in 25° C. study

| | HIAC | | | | | | | | |
|---|---|---|---|---|---|---|---|---|---|
| | (particles/ml) | | | | | | | | |
| Formulation | ≥2 μm | | | ≥10 μm | | | ≥25 μm | | |
| No. | T0 | A-3 D | A-7 D | T0 | A-3 D | A-7 D | T0 | A-3 D | A-7 D |
| F1 | 234 | 90 | 192 | 4 | 2 | 6 | 0 | 0 | 0 |
| F2 | 196 | 270 | 278 | 8 | 5 | 1 | 0 | 0 | 0 |
| F3 | 74 | 332 | 230 | 5 | 0 | 14 | 0 | 0 | 0 |
| F4 | 102 | 1642 | 3477 | 4 | 44 | 188 | 1 | 0 | 14 |
| F5 | 98 | 580 | 1706 | 0 | 10 | 35 | 0 | 1 | 1 |
| F6 | 428 | 378 | 106 | 7 | 4 | 6 | 0 | 0 | 1 |

HIAC = high accuracy;
D = day(s)

### Summary of 25° C. Study

Results showed that after 25° C. incubation for up to 7 days, no substantial change was observed in appearance, pH, antibody concentration, SEC-HPLC % Main peak, cIEF % Main peak, or subvisible particles (HIAC) for all six formulations.

### Freeze-Thaw Study

Freeze-thaw study with up to 5 cycles was used in surfactant screen study to evaluate the stability of the formulations.

### Appearance, pH, and Antibody Concentration

Table 40 shows the data summary of appearance, pH, and antibody concentration for the freeze-thaw study. After 5 cycles of freeze-thaw, all samples showed slightly yellow, slightly opalescent, and were free of particles.

The pH and antibody concentration of F1-F6 remained stable during the study, with pH value at 5.8-5.9, and antibody concentration at ˜100 mg/mL.

TABLE 40
Data summary of appearance, pH and antibody in surfactant screen freeze-thaw study

| | | | | | | | Antibody conc. | | |
|---|---|---|---|---|---|---|---|---|---|
| | | | | pH | | | (mg/mL) | | |
| Formulation | Appearance | | | | 3 | 5 | | 3 | 5 |
| No. | T0 | 3 D | 7 D | T0 | cycles | cycles | T0 | cycles | cycles |
| F1 | SY, SO, | SY, SO, | SY, SO, | 5.9 | 5.9 | 5.9 | 104.5 | 103.2 | 103.5 |
| | free of | free of | free of | | | | | | |
| | particles | particles | particles | | | | | | |
| F2 | SY, SO, | SY, SO, | SY, SO, | 5.8 | 5.9 | 5.9 | 105.0 | 102.3 | 100.6 |
| | free of | free of | free of | | | | | | |
| | particles | particles | particles | | | | | | |
| F3 | SY, SO, | SY, SO, | SY, SO, | 5.8 | 5.9 | 5.8 | 103.3 | 100.2 | 100.1 |
| | free of | free of | free of | | | | | | |
| | particles | particles | particles | | | | | | |
| F4 | SY, SO, | SY, SO, | SY, SO, | 5.8 | 5.8 | 5.8 | 100.6 | 104.3 | 104.8 |
| | free of | free of | free of | | | | | | |
| | particles | particles | particles | | | | | | |
| F5 | SY, SO, | SY, SO, | SY, SO, | 5.8 | 5.8 | 5.8 | 102.5 | 108.1 | 101.1 |
| | free of | free of | free of | | | | | | |
| | particles | particles | particles | | | | | | |
| F6 | SY, SO, | SY, SO, | SY, SO, | 5.8 | 5.9 | 5.8 | 99.0 | 103.6 | 102.2 |
| | free of | free of | free of | | | | | | |
| | particles | particles | particles | | | | | | |

SY = Slightly yellow;
SO = Slightly opalescent;
O = Opalescent;
FP = free of particles;
D = days

### SEC-HPLC

SEC-HPLC results are reported in Table 41. After 5 cycles of freeze-thaw, the Main peak percentage of all testing samples remained stable, and no substantial difference was observed among these samples.

TABLE 41
Data summary of SEC-HPLC in surfactant
screening freeze-thaw study

| | SEC-HPLC | | | | | | | | |
|---|---|---|---|---|---|---|---|---|---|
| Formulation | Main peak (%) | | | HMW (%) | | | LMW (%) | | |
| No. | T0 | 3 D | 7 D | T0 | 3 D | 7 D | T0 | 3 D | 7 D |
| F1 | 97.0 | 97.1 | 97.1 | 2.9 | 2.8 | 2.8 | 0.1 | 0.1 | 0.0 |
| F2 | 97.1 | 97.1 | 97.1 | 2.8 | 2.8 | 2.9 | 0.1 | 0.1 | 0.1 |
| F3 | 97.1 | 97.2 | 97.2 | 2.8 | 2.8 | 2.8 | 0.1 | 0.1 | 0.1 |
| F4 | 97.0 | 97.1 | 97.1 | 2.9 | 2.8 | 2.9 | 0.1 | 0.1 | 0.1 |
| F5 | 97.1 | 97.1 | 97.1 | 2.8 | 2.8 | 2.8 | 0.1 | 0.1 | 0.1 |
| F6 | 97.1 | 97.2 | 97.2 | 2.8 | 2.8 | 2.8 | 0.1 | 0.1 | 0.0 |

HMW = high molecular weight;
MLW = low molecular weight;
w = week(s);
SEC = size exclusion chromatography;
HPLC = high performance liquid chromatography;
D = day(s)

cIEF

The cIEF results are reported in Table 42. No substantial change was observed in cIEF Main peak percentage in all six formulations after 5 cycles of freeze-thaw.

TABLE 42
Data summary of cIEF in surfactant screening freeze-thaw study

| | cIEF | | | | | | | | |
|---|---|---|---|---|---|---|---|---|---|
| Formulation | Main peak (%) | | | Acidic peak (%) | | | Basic peak (%) | | |
| No. | T0 | 3C | 5C | T0 | 3C | 5C | T0 | 3C | 5C |
| F1 | 61.2 | 60.2 | 59.9 | 30.9 | 32.1 | 32.1 | 7.8 | 7.7 | 7.9 |
| F2 | 59.9 | 59.3 | 59.8 | 31.6 | 32.4 | 32.1 | 8.5 | 8.3 | 8.1 |
| F3 | 60.9 | 60.0 | 60.3 | 30.9 | 31.7 | 31.2 | 8.2 | 8.3 | 8.5 |
| F4 | 60.9 | 58.6 | 59.9 | 30.8 | 33.3 | 32.0 | 8.2 | 8.1 | 8.1 |
| F5 | 60.7 | 60.0 | 58.7 | 31.0 | 31.6 | 32.9 | 8.3 | 8.3 | 8.4 |
| F6 | 60.3 | 60.1 | 59.0 | 31.0 | 31.8 | 33.2 | 8.7 | 8.0 | 7.8 |

cIEF = capillary isoelectric focusing;
C = cycle(s)

### Non Reduced Caliper-SDS

Non-reduced Caliper-SDS results are reported in Table 43. The non-reduced Caliper-SDS purity percentage of all testing samples remained stable, and no substantial difference was observed among these samples during the study.

TABLE 43
Data summary of non-reduced Caliper-SDS
in surfactant screening freeze-thaw study

| | |
|---|---|
| Formulation | Purity % Caliper-SDS (non-reduced) |

| No. | T0 | 3 Cycles | 5 Cycles |
|---|---|---|---|
| F1 | 97.7 | 98.0 | 97.8 |
| F2 | 97.8 | 97.9 | 97.9 |
| F3 | 97.7 | 97.8 | 97.9 |
| F4 | 97.8 | 98.0 | 97.9 |
| F5 | 97.7 | 98.0 | 98.3 |
| F6 | 97.9 | 97.8 | 97.8 |

SDS = sodium dodecyl sulfate

### Reduced Caliper-SDS

Reduced Caliper-SDS results are reported in Table 44. The reduced Caliper SDS purity percentage of all testing samples remained stable, and no substantial difference was observed among these samples during the study.

TABLE 44
Data summary of reduced Caliper-SDS in
surfactant screening freeze-thaw study

| | |
|---|---|
| Formulation | HC + LC % Caliper-SDS (Reduced) |

| No. | T0 | 3 cycles | 5 cycles |
|---|---|---|---|
| F1 | 99.2 | 99.3 | 99.3 |
| F2 | 99.2 | 99.3 | 99.3 |
| F3 | 99.2 | 99.3 | 99.3 |
| F4 | 99.3 | 99.4 | 99.3 |
| F5 | 99.3 | 99.3 | 99.3 |
| F6 | 99.3 | 99.3 | 99.3 |

SDS = sodium dodecyl sulfate;
HC = heavy chain;
LC = light chain

### Subvisible Particle (HIAC)

Table 45 shows the data summary of subvisible particle (HIAC) testing result for freeze-thaw study in surfactant screening. No substantial change was observed in subvisible particles in six formulations after 5 cycles of freeze-thaw.

TABLE 45
Data summary of subvisible particle (HIAC)
in surfactant screening freeze-thaw study

| | HIAC | | | | | | | | |
|---|---|---|---|---|---|---|---|---|---|
| | (particles/ml) | | | | | | | | |
| | ≥2 μm | | | ≥10 μm | | | ≥25 μm | | |
| Formulation | | 3 | 5 | | 3 | 5 | | 3 | 5 |
| No. | T0 | cycles | cycles | T0 | cycles | cycles | T0 | cycles | cycles |
| F1 | 234 | 483 | 1165 | 4 | 18 | 25 | 0 | 0 | 0 |
| F2 | 196 | 634 | 1356 | 8 | 12 | 34 | 0 | 0 | 0 |
| F3 | 74 | 580 | 840 | 5 | 29 | 9 | 0 | 5 | 3 |
| F4 | 102 | * | 760 | 4 | * | 9 | 1 | * | 0 |
| F5 | 98 | 940 | 1096 | 0 | 17 | 14 | 0 | 0 | 3 |
| F6 | 428 | 958 | 825 | 7 | 58 | 18 | 0 | 18 | 0 |

*Sample amount not enough for HIAC testing due to vial breakage during freeze-thaw.

### Summary of Freeze-Thaw Study

Results showed that after up to 5 cycles of freeze-thaw, no substantial change was observed in appearance, pH, antibody concentration, SEC-HPLC % Main peak, cIEF % Main peak, or subvisible particles (HIAC) for all six formulations. 40° C. study Two weeks incubation at 40° C. was used in a surfactant screen study to evaluate the stability of these six formulations.

### Appearance, pH, and Antibody Concentration

Table 46 shows the data summary of appearance, pH, and antibody concentration for the 40° C. study. After 2 weeks of incubation at 40° C., all samples remained slightly yellow, slightly opalescent, and free of particles.

The pH and antibody concentration of F1-F6 remained stable during the study, with pH value at 5.8-5.9, and antibody concentration at ˜100 mg/mL.

TABLE 46
Data summary of appearance, pH and antibody
in surfactant screen 40° C. study

| | | | | | | | Antibody | | |
|---|---|---|---|---|---|---|---|---|---|
| Formu- | | | | pH | | | conc. (mg/mL) | | |
| lation | Appearance | | | | 1 | 2 | | 1 | 2 |
| No. | T0 | 1 w | 2 w | T0 | w | w | T0 | w | w |
| F1 | SY, SO, | SY, SO, | SY, SO, | 5.9 | 5.9 | 5.9 | 104.5 | 106.0 | 104.8 |
| | free of | free of | free of | | | | | | |
| | particles | particles | particles | | | | | | |
| F2 | SY, SO, | SY, SO, | SY, SO, | 5.8 | 5.9 | 5.9 | 105.0 | 112.1 | 105.1 |
| | free of | free of | free of | | | | | | |
| | particles | particles | particles | | | | | | |
| F3 | SY, SO, | SY, SO, | SY, SO, | 5.8 | 5.8 | 5.9 | 103.3 | 103.9 | 104.6 |
| | free of | free of | free of | | | | | | |
| | particles | particles | particles | | | | | | |
| F4 | SY, SO, | SY, SO, | SY, SO, | 5.8 | 5.8 | 5.8 | 100.6 | 104.1 | 107.7 |
| | free of | free of | free of | | | | | | |
| | particles | particles | particles | | | | | | |
| F5 | SY, SO, | SY, SO, | SY, SO, | 5.8 | 5.9 | 5.8 | 102.5 | 107.9 | 103.0 |
| | free of | free of | free of | | | | | | |
| | particles | particles | particles | | | | | | |
| F6 | SY, SO, | SY, SO, | SY, SO, | 5.8 | 5.8 | 5.8 | 99.0 | 101.8 | 108.6 |
| | free of | free of | free of | | | | | | |
| | particles | particles | particles | | | | | | |

SY = Slightly yellow;
SO = Slightly opalescent;
O = Opalescent;
FP = free of particles;
w = week(s)

### SEC-HPLC

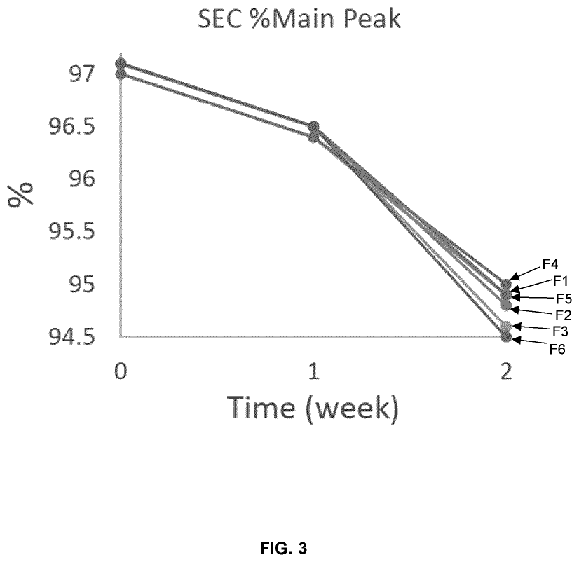

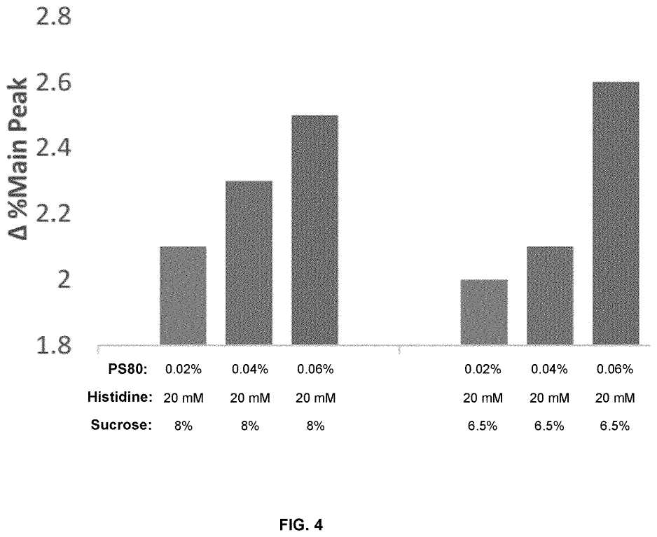

SEC-HPLC of surfactant screening 40° C. study results are reported in Table 47. After 2 weeks of incubation at 40° C., the Main peak percentage decrease was observed in all six samples, which is illustrated in FIG. 3. FIG. 4 shows the different decrease of % Main peak among these six formulations, and as PS80 concentration increased, more substantial decrease can be observed in the SEC % Main peak.

TABLE 47
Data summary of SEC-HPLC in 40°
C. study of surfactant screening

| | SEC-HPLC | | | | | | | | |
|---|---|---|---|---|---|---|---|---|---|
| Formu- | | | | HMW (%) | | | LMW (%) | | |
| lation | Main peak (%) | | | | 1 | 2 | | 1 | 2 |
| No. | T0 | 1 w | 2 w | T0 | w | w | T0 | w | w |
| F1 | 97.0 | 96.4 | 94.9 (2.1↓) | 2.9 | 3.4 | 3.4 | 0.1 | 0.2 | 1.7 |
| F2 | 97.1 | 96.5 | 94.8 (2.3↓) | 2.8 | 3.3 | 3.5 | 0.1 | 0.2 | 1.7 |
| F3 | 97.1 | 96.5 | 94.6 (2.5↓) | 2.8 | 3.3 | 3.7 | 0.1 | 0.2 | 1.8 |
| F4 | 97.0 | 96.4 | 95.0 (2.0↓5 | 2.9 | 3.3 | 3.4 | 0.1 | 0.2 | 1.6 |
| F5 | 97.1 | 96.5 | 94.9 (2.2↓4 | 2.8 | 3.3 | 3.5 | 0.1 | 0.2 | 1.6 |
| F6 | 97.1 | 96.5 | 94.5 (2.6↓4 | 2.8 | 3.3 | 3.7 | 0.1 | 0.2 | 1.8 |

HMW = high molecular weight;
MLW = low molecular weight;
w = week(s);
SEC = size exclusion chromatography;
HPLC = high performance liquid chromatography

cIEF

The cIEF results of surfactant screening 40° C. study are reported in Table 48. Substantial % Main peak decrease (˜12%) was observed in all formulations after incubation at 40° C. for 2 weeks, and no obvious difference was observed among six formulations.

TABLE 48
Data summary of cIEF in 40° C. study of surfactant screening

| Formu- | cIEF | | | | | | | | |
|---|---|---|---|---|---|---|---|---|---|
| lation | Main peak (%) | | | Acidic peak (%) | | | Basic peak (%) | | |
| No. | T0 | 1 w | 2 w | T0 | 1 w | 2 w | T0 | 1 w | 2 w |
| F1 | 61.2 | 54.6 | 49.3 | 30.9 | 37.9 | 43.5 | 7.8 | 7.5 | 7.2 |
| F2 | 59.9 | 53.2 | 48.0 | 31.6 | 38.7 | 44.9 | 8.5 | 8.1 | 7.1 |
| F3 | 60.9 | 54.5 | 48.3 | 30.9 | 37.9 | 44.7 | 8.2 | 7.6 | 7.0 |
| F4 | 60.9 | 53.4 | 49.3 | 30.8 | 39.3 | 43.4 | 8.2 | 7.3 | 7.3 |
| F5 | 60.7 | 54.3 | 49.1 | 31.0 | 37.8 | 43.7 | 8.3 | 7.8 | 7.3 |
| F6 | 60.3 | 54.7 | 47.8 | 31.0 | 37.9 | 45.5 | 8.7 | 7.4 | 6.7 |

cIEF = capillary isoelectric focusing;
w = week(s)

### Non Reduced Caliper-SDS

Non-reduced Caliper-SDS results of surfactant screening 40° C. study are reported in Table 49. The non-reduced CE-SDS purity decreased about 1.8% and no substantial difference was observed among these samples during the study.

TABLE 49
Data summary of non-reduced Caliper-SDS
in surfactant screening 40° C. study

| | |
|---|---|
| Formulation | Purity % Caliper-SDS (non-reduced) |

| No. | T0 | 1 week | 2 weeks |
|---|---|---|---|
| F1 | 97.7 | 96.9 | 96.0 |
| F2 | 97.8 | 96.9 | 96.2 |
| F3 | 97.7 | 97.0 | 95.9 |
| F4 | 97.8 | 96.9 | 96.0 |
| F5 | 97.7 | 96.8 | 95.9 |
| F6 | 97.9 | 96.9 | 96.1 |

SDS = sodium dodecyl sulfate;
w = week(s)

### Reduced Caliper-SDS

Reduced Caliper-SDS results are reported in Table 50. No substantial change was observed in reduced Caliper SDS among these samples during the study.

TABLE 50
Data summary of reduced Caliper-SDS
in surfactant screening 40° C. study

| | |
|---|---|
| Formulation | HC + LC % Caliper-SDS (Reduced) |

| No. | T0 | 1 week | 2 weeks |
|---|---|---|---|
| F1 | 99.2 | 98.9 | 98.7 |
| F2 | 99.2 | 99.3 | 98.7 |
| F3 | 99.2 | 99.0 | 99.4 |
| F4 | 99.3 | 99.0 | 98.7 |
| F5 | 99.3 | 98.9 | 98.8 |
| F6 | 99.3 | 98.9 | 98.7 |

SDS = sodium dodecyl sulfate;
HC = heavy chain;
LC = light chain

### Sub Visible Particle (HIAC)

Table 51 shows the data summary of subvisible particle (HIAC) testing result for 40° C. study in surfactant screening. No substantial change was observed in subvisible particles in six formulation after 2 weeks incubation at 40° C.

TABLE 51
Data summary of subvisible particle (HIAC) in 25° C. study

| | HIAC | | | | | |
|---|---|---|---|---|---|---|
| | (particles/ml) | | | | | |
| | ≥2 μm | | ≥10 μm | | ≥25 μm | |
| Formulation No. | T0 | 2 w | T0 | 2 w | T0 | 2 w |
| F1 | 234 | 3226 | 4 | 95 | 0 | 5 |
| F2 | 196 | 1835 | 8 | 67 | 0 | 3 |
| F3 | 74 | 763 | 5 | 12 | 0 | 0 |
| F4 | 102 | 1622 | 4 | 61 | 1 | 2 |
| F5 | 98 | 1707 | 0 | 45 | 0 | 3 |
| F6 | 428 | 138 | 7 | 6 | 0 | 0 |

### Summary of 40° C. Study

Results show that after up to 2 weeks incubation at 40° C., no substantial change was observed in appearance, pH, antibody concentration, Caliper reduced, or subvisible particles (HIAC) for all six formulations. Substantial decrease in SEC-HPLC % Main peak and cIEF % Main peak can be observed after 40° C. incubation. The increase in the PS80 concentration from 0.02% to 0.06% resulted in SEC % Main peak decrease after incubation at 40° C. for 2 weeks.

### Summary

In the agitation study, 25° C. study, and freeze-thaw study, no substantial difference was observed in appearance, pH, antibody concentration, subvisible particles, SEC purity, cIEF purity, or Caliper-SDS result.

In the 40° C. stress study, decrease in SEC % Main peak and cIEF % Main peak was observed. The SEC purity of sample with PS80 declined along with the PS80 increased, so a lower concentration of PS80 is recommended. As the range of 0.02% and 0.04% PS80 was studied, a concentration of 0.03% PS80 is recommended for the formulation.

Overall, target formulation for the anti-TTR antibody was 20 mM histidine buffer, 8% (w/v) sucrose, and 0.03% (w/v) PS80 at pH 5.8.

### Example 2. Stability Studies of the Anti-TTR Monoclonal Antibody Formulation

As outlined in detail in Example 1, above, formulation development of NI006/ALXN2220 included studies designed to select a buffer system and excipients to stabilize the protein. The formulation was developed to prevent product loss, as well as minimize the purity and bioactivity decline against stresses encountered during production, storage, shipping, and handling.

A pH buffer screening study was performed to determine the optimal buffer system for drug product formulation. 20 mM histidine buffer, at pH 5.8 was chosen as the final buffer system.

Different types of excipients, including disaccharides (such as sucrose and trehalose), amino acids (such as L-arginine HCl), polyhydric alcohol (such as sorbitol), and surfactants (such as polysorbate 80) were evaluated through excipient studies. Samples were incubated at 40° C. for up to 4 weeks. The thermal stability, formation of insoluble aggregates, and purity were monitored. Sucrose and polysorbate 80 were chosen as the optimal excipients for NI006/ALXN2220 formulation, as they were shown to minimize the decrease of SEC, cIEF and caliper purity, thus retaining product purity.

In an excipient concentration screening study, finally three different polysorbate 80 concentrations (0.02%, 0.04% and 0.06% (w/v)), and two sucrose concentrations (6.5% and 8% (w/v)) were tested. Three stress conditions were used in the screening: agitation, thermal and freeze-thaw. In agitation study, the samples were placed at 25° C., with or without agitation at 200 rpm for up to 7 days. In thermal study, the samples were placed at 40° C. for up to 2 weeks. And in freeze-thaw study, the samples were frozen and thawed for up to 5 cycles. Appearance, pH, protein concentration, number of sub-visible particles, and purity were evaluated.

A polysorbate 80 concentration of 0.03% (w/v) was chosen as the surfactant strength, since subvisible particle formation was effectively suppressed and high SEC purity was maintained. A comparison study was conducted to compare two formulations with 6.5% or 8% (w/v) sucrose concentration. No substantial differences, between formulations of 6.5% and 8% (w/v) sucrose, were observed in DSC (Differential Scanning Calorimeter), appearance, pH, protein concentration, SEC, cIEF, CE-SDS (non-reduced & reduced), sub-visible particles, and potency after incubation at 40° C. for 1 month or incubation at 25° C. for 3 months.

The final formulation developed is NI006/ALXN2220 at a target concentration of 50 mg/mL in 20 mM histidine buffer, 8% (w/v) sucrose, 0.03% (w/v) polysorbate 80, and a pH of 5.8. The excipients were selected according to their stabilizing effect on the drug product. L-Histidine and L-Histidine monohydrochloride, at a concentration of 20 mM, stabilize the pH in the liquid state. Sucrose, at a concentration of 8% (w/v), modifies the osmolality to be isotonic, stabilizes the NI006/ALXN2220 protein against aggregate formation in the liquid state, and serves as a cryo-protectant during freeze/thaw. Polysorbate 80, at a concentration of 0.03% (w/v), was chosen to stabilize NI006/ALXN2220 protein against surface-induced protein denaturation or aggregation in the liquid state.

### Manufacturing Process Development

The drug product manufacturing process consists of drug substance thawing, pooling and mixing, sterile filtration, aseptic filling, stoppering, capping, visual inspection, and bulk packaging. Sterile filtration was chosen as the method to obtain the sterile drug product and is performed by using two series connected sterile filters (0.22 μm, PVDF). Prior to and after sterile filtration, water bubble point testing for the filters is performed to assure filter integrity. The compatibility of the drug product with the contacting components on the filling line, the impact of shear stress caused by the peristaltic pump, and stability under light exposure have been evaluated to mitigate potential adverse impact on product quality attributes during manufacturing. A non-clinical lot (lot 201901004) and three clinical lots (lot 201903038, lot 201904050 and 20200801) have been filled. The non-clinical lot and clinical lots used the same fill volume, container closure system, sequence of unit operations, and storage conditions. No significant changes were made to the drug product manufacturing process between the non-clinical lot and clinical lots. Minor differences are described below:
A 2 L scale was used for the non-clinical lot and 14 L scale was used for the clinical lots.
The formulation used for the non-clinical lot 201901004 was 50 mg/mL in 20 mM histidine buffer, 8% sucrose (w/v), and 0.03% polysorbate 80 (w/v), pH 5.8. The formulation used for clinical lots 201903038 and 201904050 was 50 mg/mL in 20 mM histidine buffer, 6.5% sucrose (w/v), and 0.03% polysorbate 80 (w/v), pH 5.8. The formulation used for the clinical lots 20200801 was 50 mg/mL in 20 mM histidine buffer, 8% sucrose (w/v), and 0.03% polysorbate 80 (w/v), pH 5.8.
A 5 L glass bottle was used for pooling and mixing of the drug substance for the non-clinical lot, and a 50 L mixing bag was used for pooling and mixing of the drug substance for the clinical lots.

### Bulk Drug Substance Thawing, Pooling and Mixing

Frozen drug substance stored in 2 L PETG bottles is thawed at room temperature (18-24° C.) in a room protected from light. After complete thawing, the drug substance is pooled into a 50 L mixing bag and stirred at an appropriate speed so that movement is observed without generating foam. The mixing time is controlled at 15-20 minutes. Prior to sterile filtration, samples are taken for pH, protein concentration, osmolality, and bioburden.

### Sterile Filtration

The bulk drug substance is aseptically filtered in a Grade A environment via a peristaltic pump through two series connected 0.22 μm sterile filters into a sterile 20 L single use bag. Prior to and after sterile filtration, filter integrity testing is performed on both filters.

### Aseptic Filling

Aseptic filling is performed inside the RABS unit, which fully encloses the filler and provides a Grade A environment. The RABS unit separates the operator from the aseptic interior. All filling components are autoclaved and aseptically assembled. Sterile, depyrogenated 2 mL (2R) glass vials are filled to a target volume of 2.25 mL. Fill weight checks are performed periodically during the filling process to ensure the fill weight is 2.233-2.442 g/vial.

### Stoppering

Filled vials are automatically stoppered with 13 mm rubber stoppers inside the RABS unit. The stoppers are steam sterilized at 122° C. for 30 minutes.

### Capping

The stoppered vials are transferred to the capping machine via conveyor belt under Grade A laminar flow protection. The stoppered vials are capped with 13 mm plastic aluminum flip-off caps. The caps are steam sterilized at 122° C. for 30 minutes.

### Visual Inspection

A manual 100% visual inspection is performed on the capped vials by production personnel, followed by a statistically based AQL (acceptable quality limit) inspection by Quality Assurance. Release and stability samples are taken after visual inspection.

### Bulk Packaging and Storage

The filled drug product vials are then bulk packaged and labelled. Bulk packaged drug product vials are stored at 2-8° C.

### Container Closure System

The container closure system for the drug product is a 2 mL (2R) Type I glass vial sealed with a 13 mm rubber stopper and a 13 mm flip-off aluminum cap. The components were selected for their durability to sterilization and depyrogenation processes, as well as non-reactive contact surfaces that are optimally compatible with proteins. The compatibility of the container closure system with the drug product is evaluated by the accelerated and long-term stability studies presented below.

The integrity of the container closure system was demonstrated by the dye ingression test. Drug product vials were immersed under colored dye and held under vacuum before depressurized and the infiltration of the dye into the vials examined. The container and closure system achieved 100% air tightness. Container closure integrity testing (CCIT) is performed annually in the stability program using a non-destructive vacuum decay method. The product contact materials, the glass vial and rubber stopper, have been tested per USP and Ph. Eur. requirements and are suitable for parenteral use. Compliance is verified on the Certificate of Conformance provided by the vendor with each lot of vials and stoppers.

### Analytical Procedures

### Color

Color complies with Ph. Eur. 2.2.2. Chromatic aberration method is used for color measurement.

### Clarity

Clarity complies with Ph. Eur. 2.2.1. Light scattering method is used for clarity measurement.

pH

Complies with USP <791> and Ph. Eur. 2.2.3. Potentiometric determination method is used for pH measurement.

### Osmolality

Complies with USP <785> and Ph. Eur. 2.2.35. The osmolality is determined indirectly by the measurement of the depression of freezing point of the solution.

iCIEF

Whole column Imaging Capillary Isoelectric Focusing (iCIEF) is an identity and purity analysis method used to separate proteins according to isoelectric points (pI) and to monitor the percentage of charge variant species in a protein sample. pI is an intrinsic property of a specific protein molecule and is the pH at which the protein molecule does not carry any net electrical charge. Under an external electric field, charge variants move along a continuous pH gradient formed by ampholytes and stop where the pH equals its pI. At that pI, the protein carries no net electrical charge and is not drawn by either of the electrodes. Therefore, different monoclonal antibody species with different pI values are separated and focused at different positions. The pI values and relative abundance of the resolved peaks can be identified and quantified using chromatographic software.

To meet the acceptance criteria of “conforms to profile of the reference standard”, the electropherogram should show comparable peak profile to the electropherogram of the reference standard. In addition, the difference of pI values of the main peak between tested sample and the reference standard (average) should be no more than 0.2.

### SEC-HPLC

Size Exclusion Chromatography-High Performance Liquid Chromatography (SEC-HPLC) is a purity analysis method that separates proteins based on their sizes. The stationary phase is composed of inert particles packed into a dense three-dimensional matrix. The particles have small pores which only allow species below a certain size to enter. Larger molecules simply pass by the pores as they are too large to enter the pores. Larger molecules therefore flow through the column quicker than smaller molecules; the smaller the molecule, the longer the retention time. Following separation, the relative percentages of High Molecular Weight (HMW) species, monomer, and Low Molecular Weight (LMW) species are quantified via UV detection.

### CE-SDS (Reduced)

Reduced Capillary Electrophoresis-Sodium Dodecyl Sulfate (CE-SDS) is a purity analysis method that separates proteins based on their electrophoretic mobility, where smaller size proteins move faster than larger size proteins. In this method, the test samples are denatured by heating in the presence of SDS. The samples are reduced by the addition of reducing agent beta-mercaptoethanol (BME) to the sample solution. Separation is performed though an uncoated capillary and the protein samples are detected with a photo diode array (PDA) detector at 220 nm. Results are reported as percent purity.

### CE-SDS (Non-Reduced)

CE-SDS (Non-reduced) is a purity analysis method that separates proteins based on their electrophoretic mobility, where smaller size proteins move faster than larger size proteins. In this method, the test samples are denatured by heating in the presence of SDS. The alkylating reagent N-Ethylmaleimide (NEM) is added to the sample solution in order to prevent the sulfhydryls from binding to other sulfhydryls. Separation is performed through an uncoated capillary and the protein samples are detected with a PDA detector at 220 nm. Results are reported as percent purity.

### Bioburden

Bioburden testing is performed by membrane filtration based on USP <61> and Ph. Eur. 2.6.12. 10 mL of drug substance sample is filtered through the sterile surface of a 0.45 μm membrane. The filter membrane is then transferred onto a culture plate of Soybean-Casein Digest Agar for determination of total aerobic microbial count (TAMC). Another filter membrane used for 10 mL drug substance sample filtration is transferred onto a culture plate of Sabouraud Dextrose Agar for determination of total combined yeasts and molds count (TYMC).

### Endotoxin

Bacterial endotoxin testing is performed by kinetic turbidimetric assay and is established based on USP <85> and Ph. Eur. 2.6.14. The endotoxin generated by gram-negative bacteria is detected using amebocyte lysate from the horseshoe crab, which coagulates with endotoxin. By establishing the correlation between endotoxin concentration and the time needed to reach a predetermined absorbance of the reaction mixture, or the rate of turbidity development, the endotoxin concentration can be calculated.

### ELISA (Binding Assay)

An ELISA method is used to assess the binding potency of the NI006/ALXN2220 antibody. Samples, control, and reference standard at appropriate dilutions are loaded onto misfolded-TTR (antigen for NI006/ALXN2220) coated half-area 96 well plate(s). After washing plate, horseradish peroxidase (HRP)-conjugated goat anti-human IgG is added to the wells, allowing interaction with bound NI006/ALXN2220 antibody captured by misfolded-TTR. After a final wash step, TMB substrate solution is loaded into the wells. TMB specifically reacts with peroxide in the presence of peroxidase and produces a colorimetric signal that is proportional to the amount of NI006/ALXN2220 protein bound to the wells. The color development is stopped and optical density is measured at 450 nm (minus 560 nm for wavelength correction).

Sample and reference standard dose response curves are plotted according to a 4 parameter logistic (auto-estimate) regression model using SoftMax Pro GxP software. Individual EC50 values are calculated for samples and reference standards. Relative binding activity of the sample is calculated using the following formula:

$$Relative binding activity of the sample ( \% ) = ( EC 50 of the reference standard / EC 50 of the sample ) \times 100 \%$$

### Protein Concentration

Protein in solution absorbs ultraviolet light at a wavelength of 280 nm, due to the presence of aromatic amino acids in the protein molecule. According to Beer-Lambert law, the absorbance (A) of a protein solution at a fixed wavelength is related to the protein concentration (C), the cell path length (1), and the extinction coefficient (E) of the protein as follows: A=C I ε. Unlike traditional UV-Vis methods that rely on a single absolute absorbance value, the slope spectroscopy method uses section data (absorbance vs. path length) to determine a slope value for quantitation of sample concentration using the slope spectroscopy equation (Slope=ε C) which is derived from the Beer-Lambert law.

### Cell-Based Assay

THP-1 is a human monocytic cell line. NI006/ALXN2220 is an antibody against misfolded TTR. The bioactivity of NI006/ALXN2220 is to stimulate THP-1 cell to produce IL-8 by binding the mis-TTR in cell culture. Briefly, approximately 2×104 cells per well of THP-1 cells in assay medium are seeded into a 96-well cell culture plate at 100 μL/well, then a mixture of serial dilutions of NI006/ALXN2220 mAb standard (final concentration: 2000-0.039 g/mL) and misfolded-TTR (final concentration: 10 μg/mL) are loaded into duplicate wells at 100 μL/well. After 20-24 hours of incubation at 37° C. with 5% CO2, IL-8 production is measured using human IL-8 ELISA kit.

### Compatibility Studies

The compatibility of NI006/ALXN2220 with the clinical in-use materials was evaluated. The compatibilities with the following clinical administration settings were assessed:
Bag and infusion line under PVC setting—the IV bag, IV line, and filter are composed of PVC material
Bag and infusion line under non-PVC setting—the IV bag, IV line, and filter are composed of non-PVC material
Syringe under PVC setting—the perfusor line and filter are composed of PVC material
Syringe under non-PVC setting—the perfusor line and filter are composed of non-PVC material

Three concentrations (1.0 mg/mL, 20.0 mg/mL, and 50.0 mg/mL) at 2-8° C. for 24 hours followed by 25° C. for 6 hours (30 hours in total) were tested. Saline and glucose were used as the diluents for the concentration of 1.0 mg/mL and 20.0 mg/mL. Results are shown in Tables 52 and 53.

TABLE 52
Results of In-use Compatibility Studies.

| | | | | | Protein concentration | |
|---|---|---|---|---|---|---|
| | | | Appearance | | (mg/ml) | |
| Dose | Diluent | Contact Material | T0 | T-30 hr | T0 | T-30 hr |
| Low dose | Saline | Bag and infusion | Cl, SO, many | Cl, SO, many | 1.0 | 1.0 |
| (1.0 mg/ml) | | line under PVC | white dots | white dots | | |
| | | setting | | | | |
| | | Bag and infusion | Cl, SO, many | Cl, SO, many | 1.0 | 1.0 |
| | | line under non- | white dots | white dots | | |
| | | PVC setting | | | | |
| | | Syringe under | Cl, SO, many | Cl, SO, many | 0.9 | 1.0 |
| | | PVC setting | white dots | white dots | | |
| | | Syringe under | Cl, SO, many | Cl, SO, many | 0.9 | 1.0 |
| | | non-PVC setting | white dots | white dots | | |
| | Glucose | Bag and infusion | Cl, C, FoP | Cl, C, FoP | 1.0 | 1.0 |
| | | line under PVC | | | | |
| | | setting | | | | |
| | | Bag and infusion | Cl, C, FoP | Cl, C, FoP | 1.0 | 1.0 |
| | | line under non- | | | | |
| | | PVC setting | | | | |
| | | Syringe under | Cl, C, FoP | Cl, C, FoP | 1.0 | 0.9 |
| | | PVC setting | | | | |
| | | Syringe under | Cl, C, FoP | Cl, C, FoP | 1.0 | 0.9 |
| | | non-PVC setting | | | | |
| Mid dose | Saline | Syringe under | Cl, SO, FoP | SY, SO, | 19.9 | 19.7 |
| (20 mg/ml) | | PVC setting | | many white | | |
| | | | | dots | | |
| | | Syringe under | Cl, SO, FoP | SY, SO, | 19.8 | 19.5 |
| | | non-PVC setting | | many white | | |
| | | | | dots | | |
| | Glucose | Syringe under | Cl, C, FoP | Cl, C, FoP | 19.9 | 19.6 |
| | | PVC setting | | | | |
| | | Syringe under | Cl, C, FoP | Cl, C, 1 | 19.7 | 19.8 |
| | | non-PVC setting | | particle | | |
| High dose | NA | Bag and infusion | SY, SO, FoP | SY, SO, FoP | 49.0 | 48.7 |
| (50 mg/ml) | | line under PVC | | | | |
| | | setting | | | | |
| | NA | Bag and infusion | SY, SO, FoP | SY, SO, FoP | 49.5 | 48.9 |
| | | line under non- | | | | |
| | | PVC setting | | | | |

Cl: colorless;
SY: slightly yellow;
C: clear;
FoP: free of visible particles;
SO: slightly opalescent
¹ The concentration is the same as the NI006/ALXN2220 drug product concentration.

TABLE 53
Results of In-use Compatibility Studies (Continued)

| | | | | | Sub-visible Particles | | ELISA (Binding | |
|---|---|---|---|---|---|---|---|---|
| | | Contact | SEC-HPLC | | (10 μm/25 μm) | | Assay) | |
| Dose | Diluent | Material | T0 | T-30 hr | T0 | T-30 hr | T0 | T-30 hr |
| Low | Saline | Bag and | 97.8 | 98.4 | 164/6 | 14/0 | 94% | 96% |
| dose (1.0 | | infusion line | | | | | | |
| mg/ml) | | under PVC | | | | | | |
| | | setting | | | | | | |
| | | Bag and | 97.8 | 98.6 | 50/1 | 19/1 | 93% | 76% |
| | | infusion line | | | | | | |
| | | under non- | | | | | | |
| | | PVC setting | | | | | | |
| | | Syringe | 97.3 | 99.1 | 57/1 | 136/5 | 86% | 81% |
| | | under PVC | | | | | | |
| | | setting | | | | | | |
| | | Syringe | 97.1 | 98.9 | 29/0 | 7/0 | 124% | 89% |
| | | under non- | | | | | | |
| | | PVC setting | | | | | | |
| | Glucose | Bag and | 99.4 | 99.4 | 5/0 | 3/0 | 112% | 100% |
| | | infusion line | | | | | | |
| | | under PVC | | | | | | |
| | | setting | | | | | | |
| | | Bag and | 99.4 | 99.4 | 8/0 | 1/0 | 91% | 118% |
| | | infusion line | | | | | | |
| | | under non- | | | | | | |
| | | PVC setting | | | | | | |
| | | Syringe | 99.4 | 99.4 | 6/0 | 6/0 | 127% | 103% |
| | | under PVC | | | | | | |
| | | setting | | | | | | |
| | | Syringe | 99.3 | 99.4 | 5/0 | 1/0 | 103% | 98% |
| | | under non- | | | | | | |
| | | PVC setting | | | | | | |
| Mid dose | Saline | Syringe | 99.3 | 99.3 | 1/1 | 41/1 | 84% | 100% |
| (20 | | under PVC | | | | | | |
| mg/ml) | | setting | | | | | | |
| | | Syringe | 99.3 | 99.3 | 7/1 | 38/1 | 98% | 82% |
| | | under non- | | | | | | |
| | | PVC setting | | | | | | |
| | Glucose | Syringe | 99.3 | 99.4 | 9/0 | 5/1 | 72% | 94% |
| | | under PVC | | | | | | |
| | | setting | | | | | | |
| | | Syringe | 99.3 | 99.4 | 4/0 | 13/1 | 59%² | 72% |
| | | under non- | | | | | | |
| | | PVC setting | | | | | | |
| High | NA¹ | Bag and | 99.3 | 99.4 | 16/0 | 4/0 | 86% | 110% |
| dose (50 | | infusion line | | | | | | |
| mg/ml) | | under PVC | | | | | | |
| | | setting | | | | | | |
| | NA¹ | Bag and | 99.3 | 99.4 | 13/1 | 4/0 | 98% | 98% |
| | | infusion line | | | | | | |
| | | under non- | | | | | | |
| | | PVC setting | | | | | | |

¹The concentration is the same as the NI006/ALXN2220 drug product concentration.
²The variability of the result is due to assay variability. All results from the in-use compatibility study are within the acceptance criteria. The result is not considered safety relevant considering that, within the short in-use timeframe, the most probable incompatibility with plastics materials would be adsorption (addressed by protein concentration) and visible particle formation (addressed by the compendial method).

In the saline group, visible particles were observed, indicating that NI006/ALXN2220 is less stable in saline. The data show that when using glucose as the diluent, no substantial changes in the appearance, protein concentration, sub-visible particle, SEC-HPLC, and ELISA binding assay were observed. NI006/ALXN2220 at concentrations of 1.0 mg/mL, 20.0 mg/mL, and 50.0 mg/mL is stable at 2-8° C. for 24 hours followed by 25° C. for 6 hours (30 hours in total). NI006/ALXN2220 is compatible with the clinical in-use materials evaluated.

A concentration at 0.15 mg/mL, 10-fold lower than the lowest dose concentration in the clinical trial, was also investigated. Variations were observed in the protein concentration and ELISA testing. The variation in the ELISA testing may be caused by the protein concentration variation, which may be caused by protein adsorption to the contacting materials. In conclusion it was decided to use 5% glucose as diluent for clinical in-use.

In the following Table 54, batch analysis data are listed exemplarily for non-clinical lot 201901004 and clinical lot 201903038.

TABLE 54
Batch Analyses

| | Acceptance | | | | | |
|---|---|---|---|---|---|---|
| Test | Criteria | | 201901004 | | 201903038 | |
| Color | Not more colored | | B7 | | B7 | |
| | than B4 | | | | | |
| Clarity | ≤18 | NTU | 5.3 | NTU | 5.9 | NTU |
| Visible Particles | Liquid, essentially | | No visible particles | | Liquid, essentially | |
| | free of visible | | present | | free of visible | |
| | particles | | | | particles | |
| Extractable | ≥2.0 | mL | ≥2.0 | mL | ≥2.0 | mL |
| Volume | | | | | | |
| Sub-visible | ≥10 μm: ≤6000 | | ≥25 μm: 0 | | ≥10 μm: 9 | |
| Particles | particles/container | | particles/container; | | particles/container | |
| | ≥25 μm: ≤600 | | ≥10 μm: 2 | | ≥25 μm: 0 | |
| | particles/container | | particles/container; | | particles/container | |
| | | | ≥5 μm: 44 | | | |
| | | | particles/container; | | | |
| | | | ≥2 μm: 346 | | | |
| | | | particles/container | | | |
| pH | 5.8 ± 0.5 | | 5.8 | | 5.8 | |
| Osmolarity | ≥240 | mOsmol/Kg | 312 | mOsmol/kg | 264 | mOsmol/kg |
| iCIEF | Conforms to profile of | | pl: 9.3; | | Conforms to profile of | |
| | the reference standard | | Main peak: 63.9%; | | the reference standard | |
| | Main peak % ≥50.0% | | Acidic peaks: 28.6%; | | Main Peak %: 64.7% | |
| | Acidic peaks % ≤40.0% | | Basic peaks: 7.5% | | Acidic peaks %: 29.4% | |
| | Basic peaks % ≤15.0% | | | | Basic peaks %: 6.0% | |
| SEC-HPLC | Main peak | | Monomer: 99.1%; | | Monomer: 99.3%; | |
| | (monomer) ≥95.0%; | | HMWS: 0.8%; | | HMWS: 0.6% | |
| | HMWS % ≤5.0% | | LMWS: 0.0% | | LMWS: 0.0% | |
| | Report LMWS % | | | | | |
| CE-SDS | Light chain + heavy | | Purity (LC + HC): 97.8% | | Purity (LC + HC): 96.9%₂ | |
| (reduced) | chain: ≥90.0% | | Light chain: 29.2%; | | | |
| | | | Heavy chain: 68.6%; | | | |
| | | | Minor species: 2.2%; | | | |
| CE-SDS | Main peak ≥90.0% | | Total pre-peaks: 1.4%; | | Main Peak: 97.8% | |
| (non-reduced) | | | Total aggregates: 0.4%; | | | |
| | | | Purity: 98.2% | | | |
| Sterility | No growth | | No growth | | No growth | |
| CCIT | Pass | | Pass | | NA | |
| Endotoxin | <4.0 | EU/mL | <0.40 | EU/mL | <0.40 | EU/mL |
| ELISA (Binding | 100 ± 50% of | | 96% relative | | 98% of reference | |
| Assay) | reference | | potency | | standard | |
| | standard | | | | | |
| Protein | 50.0 ± 5.0 | mg/mL | 49.9 | mg/mL | 49.2 | mg/mL |

| | | | |
|---|---|---|---|
| Concentration | | | |
| Polysorbate 80 | 0.030 ± 0.015% | 0.031% | 0.029% |
| Cell-based Assay | Report Result | 129% | 117% |
| (Exploratory | | | |
| Specification) | | | |

¹ Acceptance criteria are not set for the non-clinical lot; data are reported for information only.
₂This result is the average of 6 injections from a retest. The original result is 92.2% for (LC + HC).
A root cause for the original result was not identified.

As reference standard, a composition comprising 50 mg/mL of the antibody formulated in 20 mM histidine buffer, 8% (w/v) sucrose, and 0.03% (w/v) PS80 at pH 5.8 and stored at −70±10 00 in vials that were filled with 100 μl was used. Additional testing was performed to qualify the reference standard and the results are shown in Table 55.

TABLE 55
Qualification results for reference standard

| Test | Results |
|---|---|
| Protein Concentration | 50.7 mg/mL |
| ELISA (Binding Assay) | 93% relative potency |
| iCIEF | pI: 9.3; |
| | Main peak: 64.2%; |
| | Acidic peaks: 28.3%; |
| | Basic peaks: 7.5% |
| SEC-HPLC | Monomer: 99.2%; |
| | HMWS: 0.8%; |
| | LMWS: ND |
| CE-SDS (Reduced) | Light chain: 30.5%; |
| | Heavy chain: 66.8%; |
| | Minor species: 2.7%; |
| | Purity (LC + HC): 97.3% |
| CE-SDS (Non-reduced) | Total pre-peaks: 1.5%; |
| | Total aggregates: 0.2%; |
| | Purity: 98.3% |

Calibration of protein concentration and potency was conducted for the reference standard. The potency of the reference standard is assigned a value of 100% relative potency.

### Stability Studies

Non-clinical lot 201901004 and clinical lot 201903038 were placed on stability testing. The non-clinical lot (201901004) has 1 month of stress stability data, 6 months of accelerated stability data, and 18 months of long-term stability data. The clinical lot (201903038) has 1 month of stress stability data and 6 months of accelerated stability data, and 12 months of long-term stability data. Under stress conditions, both clinical lot (201903038) and nonclinical lot (201901004) show trend of significant decrease in iCIEF main peak (%) and increase in acidic peaks (%), while no significant changes were observed in other purity assays as well as ELISA binding assay.

The shelf life for the drug product is currently set at 24 months stored at 5±3° C., protected from light. The available in-use stability and compatibility data, presented above, indicate that the ready-to-use solution for infusion is stable for up to 24 hours at 2-8° C. followed by 6 hours at 25° C. after dilution with 5% glucose solution. From a microbiological perspective, the solution for infusion should be used immediately. If not used immediately, the in-use shelf life is set to 4 hours at room temperature or 24 hours at 2-8° C.

Tables 56 to 59 summarize the available stress data for the non-clinical lot 201901004 and clinical lot 201903038.

TABLE 56
Stress Stability Data for Non-Clinical Lot 201901004 at 40 ± 2° C./75 ± 5% RH

| Test | T0 | 1 Week | 2 Weeks | 1 Month |
|---|---|---|---|---|
| Color | B7 | B7 | B7 | B6 |
| Clarity | 5.3 NTU | 5.4 NTU | 6.1 NTU | 6.2 NTU |
| Visible | No visible particles | No visible particles | No visible particles | No visible particles |
| Particle | present | present | present | present |
| Sub- | ≥25 μm: 0 | ≥25 μm: 0 | ≥25 μm: 0 | ≥25 μm: 1 |
| visible | particles/container | particles/container; | particles/container; | particles/container; |
| Particles | ≥10 μm: 2 | ≥10 μm: 4 | ≥10 μm: 1 | ≥10 μm: 4 |
| | particles/container; | particles/container; | particles/container; | particles/container; |
| | ≥5 μm: 44 | ≥5 μm: 37 | ≥5 μm: 26 | ≥5 μm: 48 |
| | particles/container | particles/container; | particles/container; | particles/container; |
| | ≥2 μm: 346 | ≥2 μm: 354 | ≥2 μm: 388 | ≥2 μm: 383 |
| | particles/container | particles/container | particles/container | particles/container |
| pH | 5.8 | 5.8 | 5.9 | 5.9 |
| iCIEF | pI: 9.3; | pI: 9.3; | pI: 9.3; | pI: 9.3; |
| | Main peak: 63.9%; | Main peak: 57.5%; | Main peak: 53.3%; | Main peak: 44.4%; |
| | Acidic peaks: 28.6%; | Acidic peaks: 34.7%; | Acidic peaks: 39.7%; | Acidic peaks: 49.8%; |
| | Basic peaks: 7.5% | Basic peaks: 7.8% | Basic peaks: 7.0% | Basic peaks: 5.8% |
| SEC-HPLC | Monomer: 99.1%; | Monomer: 99.0%; | Monomer: 97.5%; | Monomer: 96.0%; |
| | HMWS: 0.8%; | HMWS: 0.9%; | HMWS: 1.0%; | HMWS: 1.2%; |
| | LMWS: 0.0% | LMWS: 0.2% | LMWS: 1.4% | LMWS: 2.8% |
| CE-SDS | Light chain: | Light chain: 29.2%; | Light chain: 29.4%; | Light chain: 29.4%; |
| (Reduced) | Heavy chain: 68.6%; | Heavy chain: 68.3%; | Heavy chain: 67.5%; | Heavy chain: 65.9%; |
| | Purity: 97.8% | Minor species: 2.4%; | Minor species: 3.1%; | Minor species: 4.8%; |
| | | Purity: 97.6% | Purity: 96.9% | Purity: 95.2% |
| CE-SDS | Total pre-peaks: 1.4%; | Total pre-peaks: 2.0%; | Total pre-peaks: 2.7%; | Total pre-peaks: 4.1%; |
| (Non- | Total aggregates: 0.4%; | Total aggregates: 0.2%; | Total aggregates: 0.3%; | Total aggregates: 0.5%; |
| reduced) | Purity: 98.2% | Purity: 97.8% | Purity: 97.0% | Purity: 95.4% |
| ELISA | 96% relative | 108% relative | 74% relative | 112% relative |
| (Binding | potency | potency | potency | potency |
| Assay) | | | | |
| Protein | 49.9 mg/mL | 49.7 mg/mL | 50.2 mg/mL | 50.0 mg/mL |
| Conc. | | | | |

TABLE 57
Stress Stability Data for Exploratory Specification for Non-Clinical
Lot 201901004 at 40 ± 2° C./75 ± 5% RH

| Test | T0 | 1 Week | 2 Weeks | 1 Month |
|---|---|---|---|---|
| Cell-based | 129% | 121% | 105% | 148% |
| Assay | | | | |

TABLE 58
Stress Stability Data for Clinical Lot 201903038 at 40 ± 2° C./75 ± 5% RH

| | Acceptance | | | | |
|---|---|---|---|---|---|
| Test | Criteria | T0 | 1 Week | 2 Weeks | 1 Month |
| Color | Not more | B7 | B7 | B7 | B6 |
| | colored | | | | |
| | than B4 | | | | |
| Clarity | ≤18 NTU | 5.9 NTU | 6.1 NTU | 6.7 NTU | 6.5 NTU |
| Visible | Liquid, | Liquid, | Liquid, | Liquid, | Liquid, |
| Particle | essentially | essentially | essentially | essentially | essentially |
| | free of | free of | free of | free of | free of |
| | visible | visible | visible | visible | visible |
| | particles | particles | particles | particles | particles |
| Sub- | ≥10 μm: ≤6000 | ≥10 μm: 9 | ≥10 μm: 6 | ≥10 μm: 12 | ≥10 μm: 8 |
| visible | particles/ | particles/ | particles/ | particles/ | particles/ |
| particle | container | container | vial | vial | vial |
| | ≥25 μm: ≤600 | ≥25 μm: 0 | ≥25 μm: 2 | ≥25 μm: 1 | ≥25 μm: 0 |
| | particles/ | particles/ | particles/ | particles/ | particles/ |
| | container | container | vial | vial | vial |
| pH | 5.8 ± 0.5 | 5.8 | 5.8 | 5.8 | 5.8 |
| iCIEF | Main peak % | Main Peak %: | Main peak %: | Main peak %: | Main peak %: |
| | ≥50.0% | 64.7% | 59.6% | 54.2% | 42.5% |
| | Acidic peaks % | Acidic peaks %: | Acidic peaks %: | Acidic peaks %: | Acidic peaks %: |
| | ≤40.0% | 29.4% | 35.1% | 40.5% | 52.9% |
| | Basic peaks % | Basic peaks %: | Basic peaks %: | Basic peaks %: | Basic peaks %: |
| | ≤15.0% | 6.0% | 5.3% | 5.3% | 5.3% |
| SEC-HPLC | Main peak | Monomer: 99.3% | Monomer: 99.2% | Monomer: 99.0% | Monomer: 97.2% |
| | (monomer) | HMWS: 0.6% | HMWS: 0.7% | HMWS: 0.7% | HMWS: 0.8% |
| | ≥95.0% | LMWS: 0.0% | LMWS: 0.1% | LMWS: 0.3% | LMWS: 2.1% |
| | HMWS % ≤5.0% | | | | |
| | Report LMWS % | | | | |
| CE-SDS | Light chain + | Light chain + | Light chain + | Light chain + | Light chain + |
| (Reduced) | heavy chain: | heavy chain | heavy chain: | heavy chain: | heavy chain: |
| | ≥90.0% | 96.9% | 96.5% | 95.8% | 95.3% |
| CE-SDS | Main | Main | Main | Main | Main |
| (Non- | peak ≥90.0% | peak: 97.8% | peak: 98.1% | peak: 97.4% | peak: 95.8% |
| reduced) | | | | | |
| ELISA | 100 ± 50% | 98% of | 84% of | 76% of | 88% of |
| (Binding | of reference | reference | reference | reference | reference |
| Assay) | standard | standard | standard | standard | standard |
| Protein | 50.0 ± 5.0 | 49.2 | 49.5 | 49.3 | 48.9 |
| Concentration | mg/mL | mg/mL | mg/ml | mg/ml | mg/ml |

TABLE 59
Stress Stability Data for Exploratory Specification for Clinical
Lot 201903038 at 40 ± 2° C./75 ± 5% RH

| Test | T0 | 1 Week | 2 Weeks | 1 Month |
|---|---|---|---|---|
| Cell-based | 117% | 136% | 111% | 81% |
| Assay | | | | |

Tables 60 to 63 summarize the available accelerated data for the non-clinical lot 201901004 and clinical lot 201903038.

TABLE 60
Accelerated Stability Data for Non-Clinical Lot 201901004 at 25 ± 2° C./60 ± 5% RH

| Test | T0 | 1 Month | 3 Months | 6 Months |
|---|---|---|---|---|
| Color | B7 | B7 | B7 | B7 |
| Clarity | 5.3 NTU | 6.1 NTU | 5.5 NTU | 6.0 NTU |
| Visible | No visible | No visible | No visible | No visible |
| particle | particles present | particles present | particles present | particles present |
| | 10 μm: 2 | 10 μm: 0 | 10 μm: 5 | ≥10 μm: 4 |
| | particles/container; | particles/container; | particles/container; | particles/container; |
| | ≥25 μm: 0 | ≥25 μm: 0 | ≥25 μm: 0 | ≥25 μm: 0 |
| | particles/container; | particles/container; | particles/container; | particles/container; |
| | ≥5 μm: 44 | ≥5 μm: 39 | ≥5 μm: 25 | ≥5 μm: 95 |
| | particles/container; | particles/container; | particles/container; | particles/container; |
| | ≥2 μm: 346 | ≥2 μm: 390 | ≥2 μm: 312 | ≥2 μm: 646 |
| | particles/container | particles/container | particles/container | particles/container |
| pH | 5.8 | 5.9 | 5.8 | 5.8 |
| iCIEF | pI: 9.3; | pI: 9.3; | pI: 9.3; | pI: 9.3; |
| | Main peak: 63.9%; | Main peak: 62.2%; | Main peak: 54.2%; | Main peak: 49.1%; |
| | Acidic peaks: 28.6%; | Acidic peaks: 30.3%; | Acidic peaks: 38.3%; | Acidic peaks: 44.3%; |
| | Basic peaks: 7.5% | Basic peaks: 7.5% | Basic peaks: 7.5% | Basic peaks: 6.6% |
| SEC-HPLC | Monomer: 99.1%; | Monomer: 99.1%; | Monomer: 98.7%; | Monomer: 96.9%; |
| | HMWS: 0.8%; | HMWS: 0.8%; | HMWS: 1.0%; | HMWS: 1.3%; |
| | LMWS: 0.0% | LMWS: 0.1% | LMWS: 0.3% | LMWS: 1.8% |
| CE-SDS | Light chain: 29.2%; | Light chain: 29.2%; | Light chain: 30.6%; | Light chain: 30.1%; |
| (Reduced) | Heavy chain: 68.6%; | Heavy chain: 68.4%; | Heavy chain: 65.8%; | Heavy chain: 64.5%; |
| | Minor species: 2.2%; | Minor species: 2.4%; | Minor species: 3.6%; | Minor species: 5.3%; |
| | Purity: 97.8% | Purity: 97.6% | Purity: 96.4% | Purity: 94.7% |
| CE-SDS | Total pre-peaks: 1.4%; | Total pre-peaks: 1.6%; | Total pre-peaks: 2.6%; | Total pre-peaks: 3.6%; |
| (Non- | Total aggregates: 0.4%; | Total aggregates: 0.3%; | Total aggregates: 0.3%; | Total aggregates: 0.4%; |
| reduced) | Purity: 98.2% | Purity: 98.1% | Purity: 97.1% | Purity: 96.0% |
| ELISA | 96% relative | 94% relative | 134% relative | 96% relative |
| (Binding | potency | potency | potency | potency |
| Assay) | | | | |
| Protein | 49.9 mg/mL | 50.1 mg/mL | 50.3 mg/mL | 51.0 mg/mL |
| Concentrati [symbol] | | | | |

[symbol]

indicates data missing or illegible when filed

TABLE 61
Accelerated Stability Data for Exploratory Specification for Non-Clinical
Lot 201901004 at 25 ± 2° C./60 ± 5% RH

| Test | T0 | 1 Month | 3 Months | 6 Months |
|---|---|---|---|---|
| Cell-based | 129% | 117% | 140% | 94% |
| Assay | | | | |

TABLE 62
Accelerated Stability Data for Clinical Lot 201903038 at 25 ± 2° C./60 ± 5% RH

| | Acceptance | | | | |
|---|---|---|---|---|---|
| Test | Criteria | T0 | 1 Month | 3 Months | 6 Months |
| Color | Report result | B7 | B7 | B7 | B7 |
| | (refer to Ph. | | | | |
| | Eur.) | | | | |
| Clarity | Report result | 5.9 NTU | 6.7 NTU | 6.5 NTU | 6.3 NTU |
| Visible | Liquid, | Liquid, | Liquid, | Liquid, | Liquid, |
| Particle | essentially | essentially | essentially | essentially | essentially |
| | free of | free of | free of | free of | free of |
| | visible | visible | visible | visible | visible |
| | particles | particles | particles | particles | particles |
| Sub- | ≥10 μm: ≤6000 | ≥10 μm: 9 | ≥10 μm: 7 | ≥10 μm: 1 | ≥10 μm: 21 |
| visible | particles/ | particles/ | particles/ | particles/ | particles/ |
| Particles | container | contain | vial | vial | vial |
| | ≥25 μm: ≤600 | ≥25 μm: 0 | ≥25 μm: 0 | ≥25 μm: 0 | ≥25 μm: 3 |
| | particles/ | particles/ | particles/ | particles/ | particles/ |
| | container | container | vial | vial | vial |
| pH | 5.8 ± 0.5 | 5.8 | 5.8 | 5.8 | 5.8 |
| iCIEF | Main peak % | Main peak %: | Main peak %: | Main peak %: | Main peak %: |
| | ≥50.0% | 64.7% | 61.1% | 54.6% | 47.8% |
| | Acidic peaks % | Acidic peaks % | Acidic peaks %: | Acidic peaks %: | Acidic peaks %: |
| | ≤40.0% | Basic peaks %: | 33.4% | 40.1% | Basic peaks %: |
| | Basic peaks % | 6.0% | Basic peaks %: | Basic peaks %: | 5.4% |
| | ≤15.0% | | 5.5% | 5.2% | |
| SEC-HPLC | Main peak | Monomer: 99.3% | Monomer: 99.3% | Monomer: 98.9% | Monomer: 97.5% |
| | (monomer) ≥95.0% | HMWS: 0.6% | HMWS: 0.6% | HMWS: 0.8% | HMWS: 0.9% |
| | HMWS % ≤5.0% | Report LMWS % | Report LMWS % | LMWS: 0.3% | LMWS: 1.7% |
| | Report LMWS % | | | | |
| CE-SDS | Light chain + | Light chain + | Light chain + | Light chain + | Light chain + |
| (Reduced) | heavy chain: ≥90.0% | heavy chain: 96.9% | heavy chain: 97.2% | heavy chain: 95.5% | heavy chain: 95.7% |
| CE-SDS | Main peak ≥90.0% | Main peak: | Main peak: | Main peak: | Main peak: |
| (Non- | | 97.8% | 98.2% | 97.3% | 96.6% |
| reduced) | | | | | |
| ELISA | 100 ± 50% of | 98% of | 92% of | 98% of | 92% of |
| (Binding | reference | reference | reference | reference | reference |
| Assay) | standard | standard | standard | standard | standard |
| Protein | 50.0 ± 5.0 mg/mL | 49.2 mg/mL | 49.2 mg/mL | 49.6 mg/mL | 49.0 mg/mL |
| Concentration | | | | | |

TABLE 63
Accelerated Stability Data for Exploratory Specification for Clinical
Lot 201903038 at 25 ± 2° C./60 ± 5% RH

| Test | T0 | 1 Month | 3 Months | 6 Months |
|---|---|---|---|---|
| Cell-based | 117% | 129% | 108% | 122% |
| Assay | | | | |

Tables 64 to 69 summarize the available long-term data for the non-clinical lot 201901004 and clinical lot 201903038.

TABLE 64
Long-term Stability Data for Non-Clinical Lot 201901004 at 5 ± 3° C. (0 to 9 months)

| Test | T0 | 3 Months | 6 Months | 9 Months |
|---|---|---|---|---|
| Color | B7 | B7 | B7 | B7 |
| Clarity | 5.3 NTU | 5.5 NTU | 5.9 NTU | 6.0 NTU |
| Visible | No visible | No visible | No visible | No visible |
| Particle | particles | particles | particles | particles |
| | present | present | present | present |
| Sub- | ≥25 μm: | ≥25 μm: | ≥25 μm: | ≥25 μm: |
| visible | 0 particles/ | 4 particles/ | 3 particles/ | 1 particles/ |
| Particles | container; | container | container | container |
| | ≥10 μm: | ≥10 μm: | ≥10 μm: 6 | ≥10 μm |
| | 2 particles/ | 8 particles/ | 6 particles/ | 15 particles/ |
| | container; | container; | container; | container; |
| | ≥5 μm: 44 | ≥5 μm: | ≥5 μm: | ≥5 μm: |
| | particles/ | 19 particles/ | 54 particles/ | 44 particles/ |
| | container; | container | container | container |
| | ≥2 μm: | ≥2 μm: | ≥2 μm: 465 | ≥2 μm: |
| | 346 particles/ | 335 particles/ | 465 particles/ | 426 particles/ |
| | container | container | container | container |
| pH | 5.8 | 5.9 | 5.8 | 5.8 |
| iCIEF | pI: 9.3; | pI: 9.3; | pI: 9.3; | pI: 9.3; |
| | Main peak: 63.9%; | Main peak: 60.8%; | Main peak: 62.8%; | Main peak: 66.3%; |
| | Acidic peaks: 28.6%; | Acidic peaks: 31.4%; | Acidic peaks: 29.2%; | Acidic peaks: 26.1%; |
| | Basic peak: 7.5% | Basic peaks: 7.8% | Basic peak: 8.1% | Basic peaks: 7.5% |
| SEC- | Monomer: 99.1%; | Monomer: 99.0%; | Monomer: 99.0%; | Monomer: 98.9%; |
| HPLC | HMWS: 0.8%; | HMWS: 0.9%; | HMWS: 0.9%; | HMWS: 1.0%; |
| | LMWS: 0.0% | LMWS: 0.1% | LMWS: 0.1% | LMWS: 0.1% |
| CE-SDS | Light chain: 29.2%; | Light chain: 30.6%; | Light chain: 30.1%; | Light chain30.4%;: |
| (Reduced) | Heavy chain: 68.6%; | Heavy chain: 67.0%; | Heavy chain: 66.7%; | Heavy chain: 66.8%; |
| | Minor species: 2.2%; | Minor species: 2.4%; | Minor species: 3.2%; | Minor species: 2.8%; |
| | Purity: 97.8% | Purity: 97.6% | Purity: 96.8% | Purity: 97.2% |
| CE-SDS | Total pre-peaks1.4%;: | Total pre-peaks: 1.5%; | Total pre-peaks: 1.6%; | Total pre- peaks: 1.6%; |
| (Non- | Total aggregates: 0.4%; | Total aggregates: 0.4%; | Total aggregates: 0.2%; | Total aggregates: 0.3%; |
| reduced) | Purity: 98.2% | Purity: 98.2% | Purity: 98.2% | Purity: 98.1% |
| CCIT | Pass | NT | NT | NT |
| ELISA | 96% relative | 124% relative | 88% relative | 99% relative |
| (Binding | potency | potency | potency | potency |
| Assay) | | | | |
| Protein | 49.9 mg/mL | 50.3 mg/mL | 50.7 mg/mL | 49.8 mg/mL |
| Conc. | | | | |
| Polysorbate | 0.031% (w/v) | NT | 0.031% (w/v) | NT |
| 80 | | | | |

TABLE 65
Long-term Stability Data for Non-Clinical Lot 201901004 at 5 ± 3° C. (11 to 18 months)

| Test | 11 Months | 12 Months | 15 Months | 18 Months |
|---|---|---|---|---|
| Color | B7 | B7 | B7 | B7 |
| Clarity | 6.1 NTU | 5.5 NTU | 5.3 NTU | 6.0 NTU |
| Visible | No visible | No visible | No visible | No visible |
| Particle | particles | particles | particles | particles |
| | present | present | present | present |
| | ≥25 μm: 0 | ≥25 μm: 1 | ≥25 μm: | ≥25 μm: |
| | particles/ | 1 particles/ | 3 particles/ | 0 particles/ |
| | container | container | container | container |
| Sub- | ≥10 μm: 16 | ≥10 μm: 4 | ≥10 μm: 13 | ≥10 μm: |
| visible | 16 particles/ | 4 particles/ | 13 particles/ | 2 particles/ |
| Particles | container | container; | container | container; |
| | ≥5 μm: 114 | ≥5 μm: | ≥5 μm: | ≥ 5 μm: 49 |
| | particles/ | 46 particles/ | 84 particles/ | 49 particles/ |
| | container | container | container | container |
| | ≥2 μm | ≥2 μm: | ≥2 μm: | ≥2 μm: |
| | 716 particles/ | 525 particles/ | 685 particles/ | 484 particles/ |
| | container | container | container | container |
| pH | 5.9 | 5.9 | 5.8 | 5.9 |
| iCIEF | pI: 9.3; | pI: 9.3; | pI: 9.3; | pI: 9.2; |
| | Main peak63.8%; | Main peak: 61.5%; | Main peak: 60.8%; | Main peak: 60.5%; |
| | Acidic peaks: 29.0%; | Acidic peaks: 31.0%; | Acidic peaks: 31.8%; | Acidic peaks: 31.5%; |
| | Basic peaks: 7.1% | Basic peaks: 7.5% | Basic peaks: 7.3% | Basic peaks: 8.0% |
| SEC- | Monomer: 99.0%; | Monomer: 99.0%; | Monomer: 98.9%; | Monomer: 98.9%; |
| HPLC | HMWS: 0.9%; | HMWS: 0.9%; | HMWS: 1.0%; | HMWS: 1.0%; |
| | LMWS: 0.1% | LMWS: 0.1% | LMWS: 0.1% | LMWS: 0.1% |
| CE-SDS | Light chain: 32.0%; | Light chain: 30.9%; | Light chain: 31.7%; | Light chain: 31.6%; |
| (Reduced) | Heavy chain: 65.2%; | Heavy chain: 66.5%; | Heavy chain: 65.6%; | Heavy chain: 65.7%; |
| | Minor species: 2.8%; | Minor species: 2.6%; | Minor species: 2.7%; | Minor species: 2.7%; |
| | Purity: 97.2% | Purity: 97.4% | Purity: 97.3% | Purity: 97.3% |
| CE-SDS | Total pre-peaks: 1.7%; | Total pre-peaks: 1.7%; | Total pre-peaks: 2.1%; | Total pre-peaks: 1.7%; |
| (Non- | Total aggregates: 0.3%; | Total aggregates: 0.3%; | Total aggregates: 0.2%; | Total aggregates: 0.3%; |
| reduced) | Purity: 98.0% | Purity: 97.9% | Purity: 97.7% | Purity: 97.9% |
| CCIT | NT | Pass | NT | NT |
| ELISA | 103% relative | 111% relative | 84% relative | 91% relative |
| (Binding | potency | potency | potency | potency |
| Assay) | | | | |
| Protein | 49.8 mg/mL | 49.8 mg/mL | 50.5 mg/mL | 51.8 mg/mL |
| Conc. | | | | |
| Polysorbate | NT | 0.030% (w/v) | NT | NT |
| 80 | | | | |

TABLE 66
Long-term Stability Data for Exploratory Specification for
Non-Clinical Lot 201901004 at 5 ± 3° C.

| Test | T0 | 3 Months | 6 Months | 9 Months | 11 Months | 12 Months | 15 Months | 18 Months |
|---|---|---|---|---|---|---|---|---|
| Cell- | 129% | 110% | 101% | 113% | 126% | 128% | 144% | 136% |
| based | | | | | | | | |
| Assay | | | | | | | | |

TABLE 67
Long-term Stability Data for Clinical Lot 201903038 at 5 ± 3° C. (0 to 6 months)

| Test | Acceptance Criteria | T0 | 3 Months | 6 Months |
|---|---|---|---|---|
| Color | Not more colored | B7 | B6 | B7 |
| | than B4 | | | |
| Clarity | ≤18 NTU | 5.9 NTU | 6.7 NTU | 6.2 NTU |
| Visible | Liquid, | Liquid, | Liquid, | Liquid, |
| Particle | essentially | essentially | essentially | essentially |
| | free of | free of | free of | free of |
| | visible | visible | visible | visible |
| | particles | particles | particles | particles |
| Sub- | ≥10 μm: ≤6000 | ≥10 μm: 9 | ≥10 μm: 0 | ≥10 μm: 98 |
| visible | particles/ | particles/ | particles/ | particles/ |
| Particles | container | container | container | container |
| | ≥25 μm: ≤600 | ≥25 μm: 0 | ≥25 μm: 0 | ≥25 μm: 43 |
| | particles/ | particles/ | particles/ | particles/ |
| | container | container | container | container |
| pH | 5.8 ± 0.5 | 5.8 | 5.8 | 5.8 |
| iCIEF | Main peak % ≥50.0% | Main peak %: 64.7% | Main peak %: 62.2% | Main peak %: 62.9% |
| | Acidic peaks % ≤40.0% | Acidic peaks %: 29.4% | Acidic peaks %: 32.3% | Acidic peaks %: 31.8% |
| | Basic peaks % ≤15.0% | Basic peaks %: 6.0% | Basic peaks %: 5.5% | Basic peaks %: 5.3% |
| SEC-HPLC | Main peak | Monomer: 99.3% | Monomer: 99.2% | Monomer: 99.3% |
| | (monomer) ≥95.0% | HMWS: 0.6% | HMWS: 0.7% | HMWS: 0.7% |
| | HMWS % ≤5.0% | LMWS: 0.0% | LMWS: 0.1% | LMWS: 0.1% |
| | Report LMWS % | | | |
| CE-SDS | Light chain + | Light chain + | Light chain + | Light chain + |
| (Reduced) | heavy chain: ≥90.0% | heavy chain: 96.9% | heavy chain: 96.8% | heavy chain: 97.4% |
| CE-SDS | Main peak ≥90.0% | Main peak: 97.8% | Main peak: 98.4% | Main peak: 98.5% |
| (Non- | | | | |
| reduced) | | | | |
| ELISA | 100 ± 50% of | 98% of | 116% of | 98% of |
| (Binding | reference | reference | reference | reference |
| Assay) | standard | standard | standard | standard |
| Protein | 50.0 ± 5.0 mg/mL | 49.2 mg/mL | 49.1 mg/mL | 48.8 mg/mL |
| Conc. | | | | |
| CCIT | Pass | Passed | Not tested | Not tested |

TABLE 68
Long-term Stability Data for Clinical Lot 201903038 at 5 ± 3° C. (9 to 12 months)

| | Acceptance | | |
|---|---|---|---|
| Test | Criteria | 9 Months | 12 Months |
| Color | Not more colored | B7 | B7 |
| | than B4 | | |
| Clarity | ≤18 NTU | 6.3 NTU | 6.5 NTU |
| Visible | Liquid, | Liquid, | Liquid, |
| Particle | essentially | essentially | essentially |
| | free of | free of | free of |
| | visible | visible | visible |
| | particles | particles | particles |
| Sub- | ≥10 μm: ≤6000 | ≥10 μm: 6 | ≥10 μm: 10 |
| visible | particles/ | particles/ | particles/ |
| Particles | container | container | container |
| | ≥25 μm: ≤600 | ≥25 μm: 1 | ≥25 μm: 0 |
| | particles/ | particles/ | particles/ |
| | container | container | container |
| pH | 5.8 ± 0.5 | 5.8 | 5.8 |
| iCIEF | Main peak % ≥50.0% | Main peak %: 62.7% | Main peak %: 61.2% |
| | Acidic peaks % ≤40.0% | Acidic peaks %: 32.2% | Acidic peaks %: 33.9% |
| | Basic peaks % ≤15.0% | Basic peaks %: 5.2% | Basic peaks %: 4.9% |
| SEC-HPLC | Main peak | Monomer: 99.2% | Monomer: 99.2% |
| | (monomer) ≥95.0% | HMWS: 0.7% | HMWS: 0.6% |
| | HMWS % ≤5.0% | LMWS: 0.1% | LMWS: 0.2% |
| | Report LMWS % | | |
| CE-SDS | Light chain + | Light chain + | Light chain + |
| (Reduced) | heavy chain: ≥90.0% | heavy chain: 96.7% | heavy chain: 96.9% |
| CE-SDS | Main peak ≥90.0% | Main peak: 98.3% | Main peak: 98.5% |
| (Non- | | | |
| reduced) | | | |
| ELISA | 100 ± 50% of | 109% of | 98% of |
| (Binding | reference | reference | reference |
| Assay) | standard | standard | standard |
| Protein | 50.0 ± 5.0 mg/mL | 49.6 mg/mL | 49.0 mg/mL |
| Concentration | | | |
| CCIT | Pass | Not tested | Pass |

TABLE 69
Long-term Stability Data for Exploratory Specification
for Clinical Lot 201903038 at 5 ± 3° C.

| Test | T0 | 3 Months | 6 Months | 9 Months | 12 Months |
|---|---|---|---|---|---|
| Cell-based | 117% | 99% | 106% | 104% | 123% |
| Assay | | | | | |

As can be derived from the stress stability data, the accelerated stability data, and long-term stability data shown in Tables 55 to 69, the tested pharmaceutical formulation is long-term stable. For example, the formulation remains liquid with no visible particles, the pH remains constant, and the monomer content as measured by SEC-HPLC does not drop below 96%, meaning that the content of HMWS and LMWS remains under 4% under all tested conditions. Furthermore, the amount of acidic species does not exceed 40% during long-term stability testing as measured by iCIEF, and ELISA binding assays showed that the antibody remains its binding capacity (it does not drop below 70% of reference standard under all conditions tested and even does not drop below 95% during long-term stability studies).

### Example 3. Characterisation of Mature NI006/ALXN2220

Antibody NI006/ALXN2220 was produced in the CHO-K1 cell line ATCC No. CCL 61 and obtained from the cell culture after culturing in a large-scale production bioreactor. The amino acid sequence of NI006/ALXN2220's mature heavy chain (HC) and light chain (LC) is set forth in SEQ ID NOs: 9 and 10, with the below-mentioned modifications. The total number of amino acids of the IgG antibody, number of amino acids of the heavy chain, and number of amino acids of the light chain are 1328, 450, and 214, respectively.

Further characterization of antibody NI006/ALXN2022 was mainly performed by standard procedures, for example by mass spectroscopy analysis. For example, liquid chromatography with tandem mass spectrometry (LC-MS/MS) analysis of fragments of NI006/ALXN2220 obtained from Lys-C and trypsin sequential digestion as well as free sulfhydryl analysis was used to identify post-translational modifications of NI006/ALXN2220. The characterization of antibody-based therapeutics via LC-MS analysis is a standard procedure and can be performed by a skilled artisan; see for example Robotham and Kelly, Approaches to the Purification, Analysis and Characterization of Antibody-Based Therapeutics (2020), 1-33.

N-glycan profiling was performed by releasing the N-glycans by using PNGase F and subsequent labelling with 2-AB, followed by HILIC (Hydrophilic Interaction Chromatography) separation and fluorescence detection (FLD) with a UPLC system. Individual N-glycans and unknown peaks were quantified by their peak area percentages relative to the total peak area.

The results are mentioned in the following:

The molecular weight of antibody NI006/ALXN2220 as determined by standard mass spectroscopy is approximately 147.1 kDa for the intact IgG1 and 144.2 kDa for the deglycosylated variant.

The monoclonal antibody NI006/ALXN2220 is an IgG1 subclass antibody, which is composed of two heavy chains of the IgG1 subclass and two light chains of the kappa subclass. The four chains are stabilized by multiple disulfide bonds. In particular, as determined by standard procedures, i.e., per Lys-C and trypsin digestion and subsequent LC-MS, at least the following disulfide bridges are present in NI006/ALXN2 220:
LC: C23-LC:C88
LC: C134-LC:C194
LC:C214-HC:C223
HC:C22-HC:C97
HC:C147-HC:C203
HC1:229-HC2:229 and HC1:232-HC2:232
HC:C264-HC:C324
HC:C370-HC:C428
(the corresponding amino acid sequences of the HC and LC are set forth in SEQ ID NOs: 9 and 10)

NI006/ALXN2220 is a glycoprotein, and the constant region of each heavy chain contains one N-linked glycan site at residue N300. During determination of the glycosylation profile, it was shown that the major N-glycan types are GOF (˜49.0%) and G1F (˜25.4%). In more detail, the following glycosylation profile (the types of sugar, the location of glycosylation site(s), etc.) has been determined for NI006/ALXN2220:

TABLE 70
Glycan Types

| Glycan | Glycan Type |
|---|---|
| 23100 | Man3 + 1F |
| 33000 | G0 − GN |
| 33100 | G0F − GN |
| 43000 | G0 |
| 43100 | G0F |
| 25000 | Man5 |
| 34100/44000 | G1F − GN/G1a |
| 44000 | G1b |
| 44100 | G1Fa |
| 44100 | G1Fb |
| 45100 | G2F |
| 45110 | G2FS1 |
| 45120 | G2FS2 |

Note:
1. The nomenclature of the glycans follows the order of HexNac-Hexose-Fucose-NeuAc-NeuGc. For example, 23000 is HexNac(2)-Hexose(3)-Fucose(0)-NeuAc(0)-NeuGc(0).
2. G1Fa and G1Fb are isomers and are grouped into G1F. G1F is calculated as the sum of G1Fa and G1Fb using the original unrounded numbers.

Furthermore, glutamine at N-terminal modified as pyro-glutamic (abundance in sample: 99.9%) acid and C-terminal lysine clipping of the heavy chain (abundance in sample: 95.8%) have been identified as the major post-translational modifications. In addition, small ratio modifications, such as methionine oxidation, asparagine deamidation and asparagine succinimide formation, have been experimentally determined as shown in Table 71:

TABLE 71
Heavy chain portion sequences

| | Se- | | SEQ | |
|---|---|---|---|---|
| | quence | | ID | |
| No. | No. | Sequence | NO: | PTMs |
| *1 | HC: | QLQLQESGPGLVK | 16 | / |
| | 1-13 | QLQLQESGPGLVK | 16 | pE(Q) |
| 2 | HC: | DTLMISR | 17 | / |
| | 252-258 | DTLMISR | 17 | Oxidation (M) |
| 3 | HC: | VVSVLTVLHQDWLNGK | 18 | / |
| | 305-320 | VVSVLTVLHQDWLNGK | 18 | Deamidation (N) |
| | | VVSVLTVLHQDWLNGK) | 18 | Succinimide |
| | | | | Formation (N) |
| 4 | HC: | GFYPSDIAVEWESNGQ | 19 | / |
| | 374-395 | PENNYK | | |
| | | GFYPSDIAVEWESNGQ | 19 | Deamidation (N) |
| | | PENNYK | | |
| #5 | HC: | SLSLSPGK | 20 | / |
| | 443-450 | SLSLSPGK | 20 | -K |
| | | SLSLSPGK | 20 | -K -G |
| | | | | Amidation (P) |

Note:
1. HC refers to heavy chain and LC refers to light chain.
2. Peptide sequences in underlined font were identified as the site of the PTMs.
3. * refers to N-terminal related peptide of heavy chain and # refers to C-terminal related peptide of heavy chain.
4. / refers to no PTM reported.
5. pE(Q) refers to the N-terminal glutamine modified as pyro-glutamic acid.
6. -K refers to loss of the C-terminal lysine.
7. -K -G Amidation(P) refers to the amidation of the C-terminal proline after the loss of the C-terminal lysine and glycine.

In summary, N-linked glycosylation of the heavy chain, N-terminal pyro-glutamic acid modified from N-terminal glutamine, and C-terminal lysine clipping of the heavy chain are the major post-translational modifications of NI006/ALXN2220

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

1. A pharmaceutical composition comprising a human anti-transthyretin (TTR) antibody or antigen-binding fragment thereof capable of binding aggregated TTR species and does not substantially recognize physiological TTR species, wherein the pharmaceutical composition comprises one or more of sucrose, polysorbate 80, and a polar excipient.

2. The pharmaceutical composition of claim 1, which is an aqueous formulation.

3. The pharmaceutical composition of claim 1 or 2, wherein the antibody binds mutated and wild-type aggregated TTR species.

4. The pharmaceutical composition of any one of claims 1 to 3, wherein the antibody does not bind to monomers and dimers of human native TTR.

5. The pharmaceutical composition of any one of claims 1 to 4, wherein the antibody binds to a TTR epitope which comprises or consists of the amino acid sequence EEEFVEGIY (SEQ ID NO: 49), GELHGLTTEEE (SEQ ID NO: 50), or WEPFA (SEQ ID NO: 51).

6. The pharmaceutical composition of any one of claims 1 to 5, wherein the pharmaceutical composition comprises from about 6% to about 9% weight per unit volume (w/v) sucrose.

7. The pharmaceutical composition of any one of claims 1 to 6, wherein the pharmaceutical composition comprises from about 6% to about 7% w/v sucrose.

8. The pharmaceutical composition of any one of claims 1 to 7, wherein the pharmaceutical composition comprises about 6.5% w/v sucrose.

9. The pharmaceutical composition of any one of claims 1 to 6, wherein the pharmaceutical composition comprises from about 7.5% to about 8.5% w/v sucrose.

10. The pharmaceutical composition of any one of claims 1 to 6, and 9, wherein the pharmaceutical composition comprises about 8% w/v sucrose.

11. The pharmaceutical composition of any one of claims 1 to 10, wherein the pharmaceutical composition comprises from about 0.001% to about 0.1% w/v polysorbate 80.

12. The pharmaceutical composition of any one of claims 1 to 10, wherein the pharmaceutical composition comprises from about 0.01% to about 0.05% w/v polysorbate 80.

13. The pharmaceutical composition of any one of claims 1 to 12, wherein the pharmaceutical composition comprises about 0.03% w/v polysorbate 80.

14. The pharmaceutical composition of any one of claims 1 to 13, wherein the pharmaceutical composition has a pH of about 5.3 to about 6.3.

15. The pharmaceutical composition of any one of claims 1 to 14, wherein the pharmaceutical composition has a pH of about 5.8 to about 5.9.

16. The pharmaceutical composition of any one of claims 1 to 15, wherein the pharmaceutical composition has a pH of about 5.8.

17. The pharmaceutical composition of any one of claims 1 to 16, wherein the pharmaceutical composition has a pH of 5.8±0.5.

18. The pharmaceutical composition of any one of claims 1 to 16, wherein the pharmaceutical composition has a pH of 5.8±0.1.

19. The pharmaceutical composition of any one of claims 1 to 18, wherein the polar excipient comprises histidine.

20. The pharmaceutical composition of any one of claims 1 to 19, wherein the polar excipient comprises from about 1 mM to about 100 mM histidine.

21. The pharmaceutical composition of any one of claims 1 to 20, wherein the polar excipient comprises about 20 mM histidine.

22. The pharmaceutical composition of any one of claims 19 to 21, wherein the histidine comprises L-histidine and/or L-histidine monohydrochloride or a pharmaceutically acceptable salt thereof.

23. The pharmaceutical composition of any one of claims 1 to 22, wherein the anti-TTR antibody or antigen-binding fragment thereof comprises a heavy chain variable (VH) region having the three complementary determining regions (CDRs) set forth in SEQ ID NOs: 1-3 and a light chain variable (VL) region having the three CDRs set forth in SEQ ID NOs: 4-6.

24. The pharmaceutical composition of claim 23, wherein the VH region comprises an amino acid sequence having at least 80% sequence identity to SEQ ID NO: 7 and the VL region comprises an amino acid sequence having at least 80% sequence identity to SEQ ID NO: 8.

25. The pharmaceutical composition of claim 23 or 24, wherein the VH region comprises the amino acid sequence of SEQ ID NO: 7 and the VL region comprises the amino acid of SEQ ID NO: 8.

26. The pharmaceutical composition of claim 23 or 24, wherein the VH region comprises the amino acid sequence of SEQ ID NO: 11 and the VL region comprises the amino acid of SEQ ID NO: 12.

27. The pharmaceutical composition of any one of claims 1 to 26, wherein the antibody comprises a human Ig constant region.

28. The pharmaceutical composition of any one of claims 1 to 27, wherein the antibody is a human IgG.

29. The pharmaceutical composition of any one of claims 1 to 28, wherein the antibody is a human IgG1 type.

30. The pharmaceutical composition of any one of claims 1 to 28, wherein the antibody is a human IgG1m3 allotype.

31. The pharmaceutical composition of any one of claims 27 to 30, wherein the antibody comprises a kappa (κ) light chain.

32. The pharmaceutical composition of any one of claims 1 to 31, wherein the antibody is NI006/ALXN2220.

33. The pharmaceutical composition of any one of claims 1 to 32, wherein the antibody comprises two heavy chains and two light chains, wherein each heavy chain is comprised of 450 amino acid residues having SEQ ID NO: 9, and each light chain is comprised of 214 amino acid residues having SEQ ID: 10.

34. The pharmaceutical composition of any one of claims 1 to 32, wherein the antibody comprises two heavy chains and two light chains, wherein each heavy chain is comprised of 449 amino acid residues having SEQ ID NO: 13, and each light chain is comprised of 214 amino acid residues having SEQ ID: 10.

35. The pharmaceutical composition of any one of claims 1 to 32, wherein the antibody comprises two heavy chains and two light chains, wherein each heavy chain is comprised of 449 amino acid residues having SEQ ID NO: 14, and each light chain is comprised of 214 amino acid residues having SEQ ID: 10.

36. The pharmaceutical composition of any one of claims 1 to 32, wherein the antibody comprises two heavy chains and two light chains, wherein each heavy chain is comprised of 448 amino acid residues having SEQ ID NO: 15 and each light chain is comprised of 214 amino acid residues having SEQ ID: 10.

37. The pharmaceutical composition of any one of claims 1 to 36, wherein the antibody is produced in CHO host cells, preferably in CHO-K1 cells and purified from the cell culture.

38. The pharmaceutical composition of any one of claims 27 to 33 and 37, wherein the heavy chain of the antibody comprises N-terminal glutaminyl cyclization.

39. The pharmaceutical composition of any one of claims 27 to 33 and 37 and 38, wherein the heavy chain comprises C-terminal lysine clipping.

40. The antibody for use in a method according to any one of claims 27 to 39, which is N-glycosylated, preferably wherein the N-linked glycan is on N300 of the heavy chain.

41. The pharmaceutical composition of any one of claims 1 to 40, wherein the anti-TTR antibody or antigen-binding fragment thereof is present at a concentration of about 1 mg/mL to about 500 mg/mL.

42. The pharmaceutical composition of any one of claims 1 to 42, wherein the anti-TTR antibody or antigen-binding fragment thereof is present at a concentration of about 1 mg/mL to about 150 mg/mL.

43. The pharmaceutical composition of any one of claims 1 to 42, wherein the anti-TTR antibody or antigen-binding fragment thereof is present at a concentration of about 50 mg/mL or about 100 mg/mL.

44. The pharmaceutical composition of any one of claims 1 to 43, wherein the pharmaceutical composition is present in a volume of about 0.1 mL to about 100 mL.

45. The pharmaceutical composition of any one of claims 1 to 44, wherein the pharmaceutical composition is present in a volume of about 1 mL to about 25 mL.

46. The pharmaceutical composition of any one of claims 1 to 45, wherein the pharmaceutical composition is present in a volume of about 2 mL or about 20 mL.

47. The pharmaceutical composition of any one of claims 1 to 46, wherein the pharmaceutical composition is formulated for intravenous injection.

48. The pharmaceutical composition of any one of claims 1 to 47, wherein the pharmaceutical composition has not been reconstituted from a lyophilized anti-TTR antibody or antigen-binding fragment thereof and/or is not further lyophilized.

49. The pharmaceutical composition of any one of claims 1 to 48, wherein the pharmaceutical composition is essentially free of sodium chloride.

50. The pharmaceutical composition of any one of claims 1 to 49, wherein the pharmaceutical composition the formulation is essentially free of a poloxamer.

51. The pharmaceutical composition of any one of claims 1 to 50, wherein the pharmaceutical composition meets at least one of the following stability criteria, and optionally may satisfy any combination of two or all three the stability criteria: remains stable at 40±2° C. and 75±5% relative humidity (RH) for at least 1 week, preferably up to 1 month; remains stable at 25±2° C./60±5% RH for at least 1 month, preferably for up to 6 months; remains stable at 5±3° C. for at least 1 month, preferably at least 18 months.

52. The pharmaceutical composition of any one of claims 1 to 51, wherein the pharmaceutical composition is characterized by one, two, three or all four stability criteria (a) to (d) in any combination:
(a) the main peak decline under heat stress conditions for 4 weeks at 40° C. and/or 12 weeks at 25° C. is less than 1% of antibody by weight as measured by Size Exclusion Chromatography (SEC)-HPLC analysis;
(b) the pharmaceutical composition shows a content of acidic species of the anti-TTR antibody or antigen-binding fragment thereof under heat stress conditions for 2 weeks at 40° C. of less than 42.5% as measured by Capillary Isoelectric Focusing (cIEF);
(c) the pharmaceutical composition shows no substantial change in the content of acidic species of the anti-TTR antibody after 3 cycles freeze thaw (−70° C. to room temperature (RT)) as measured by Capillary Isoelectric Focusing (cIEF); and/or
(d) the anti-TTR antibody or antigen-binding fragment thereof retains a binding potency to a TTR protein of at least 80% after storage for 4 weeks at 40° C. and/or of at least 70% after storage for 12 weeks at 25° C. as measured by ELISA and relative to a control.

53. The pharmaceutical composition of any one of claims 1 to 52, wherein the pharmaceutical composition shows a main peak≥50.0%, an acidic peak≤40.0%, and a basic peak≤15.0% as measured, e.g., by cIEF, a main peak (monomer)≥95.0% and High Molecular Weight Species (HMWS)≤5.0% as measured by, e.g., Size Exclusion Chromatography (SEC)-HPLC analysis, a pH 5.8±0.5, an osmolality≥240 mOsm/Kg, and an antibody concentration of 50±5.0 mg/mL.

54. The pharmaceutical composition of any one of claims 1 to 53, wherein the pharmaceutical composition is characterized by one, two, three or all four stability criteria (i) to (iv) in any combination:
(i) the main peak decline under heat stress conditions for 1 month at about 40° C., or for 6 months at about 25° C., or during long-term storage for 18 months at about 5° C. is less than 5%, preferably less than 4%, more preferably less than 3%, more preferably less than 2% as measured by SEC-HPLC;
(ii) the pharmaceutical composition shows a content of acidic species of the anti-TTR antibody or antigen-binding fragment thereof under heat stress conditions for 2 weeks at about 40° C., or for 3 months at about 25° C. of less than or about equal to about 40% as measured by cIEF;
(iii) the pharmaceutical composition shows a content of acidic species of the anti-TTR antibody or antigen-binding fragment thereof under long-term storage conditions for 12 months or 18 months at about 5° C. of less than 40%, preferably of less than 35% as measured by cIEF; and/or
(iv) the anti-TTR antibody or antigen-binding fragment thereof retains binding potency to a TTR protein of at least 80%, preferably of at least 90% after storage for 6 months at about 25° C., or after storage for 12 months or 18 months at about 5° C. as measured by ELISA and relative to a control.

55. The pharmaceutical composition of any one of claims 1 to 54, wherein the pharmaceutical composition has an osmolality of ≥240 mOsm/Kg.

56. The pharmaceutical composition of any one of claims 1 to 55, wherein the pharmaceutical composition is sterile.

57. The pharmaceutical composition of any one of claims 1 to 56, wherein the pharmaceutical composition is stable upon freezing and thawing.

58. The pharmaceutical composition of any one of claims 1 to 57, wherein the pharmaceutical composition is present in one or more vials or infusion bottles that in total comprise about 2500 mg to about 7500 mg of the anti-TTR antibody or antigen-binding fragment thereof.

59. The pharmaceutical composition of any one of claims 1 to 58, wherein the pharmaceutical composition is present in a vial or infusion bottle, preferably a vial or infusion bottle comprising a volume of 2 mL to 20 mL, such as a 2 mL or a 20 mL Type I clear glass vial.

60. The pharmaceutical composition of claim 59, wherein the vial or infusion bottle contains an approximate 12.5% volume overfill, or a total volume of approximately 2.25 mL or 22.5 mL of the anti-TTR antibody or antigen-binding fragment thereof.

61. The pharmaceutical composition of any one of claims 1 to 60, wherein:
(a) the pharmaceutical composition comprises 6.5% w/v sucrose (65 mg/mL sucrose), 0.03% w/v polysorbate 80 (0.3 mg/mL polysorbate 80), 20 mM histidine (L-Histidine 1.06 mg/mL and L-Histidine monohydrochloride 2.78 mg/mL), a pH of 5.8, and a volume of 2-50 mL; and
(b) the anti-TTR antibody or an antigen-binding fragment thereof comprises a VH region having the amino acid sequence of SEQ ID NO: 7 and VL region having the amino acid sequence of SEQ ID NO: 8.

62. The pharmaceutical composition of any one of claims 1 to 60, wherein:
(a) the pharmaceutical composition comprises 8% w/v sucrose (80 mg/mL sucrose), 0.03% w/v polysorbate 80 (0.3 mg/mL polysorbate 80), 20 mM histidine (L-Histidine 1.06 mg/mL and L-Histidine monohydrochloride 2.78 mg/mL), a pH of 5.8, and a volume of 2-50 mL; and
(b) the anti-TTR antibody or an antigen-binding fragment thereof comprises a VH region having the amino acid sequence of SEQ ID NO: 7 and VL region having the amino acid sequence of SEQ ID NO: 8.

63. The pharmaceutical composition of any one of claims 1 to 60, wherein:
(a) the pharmaceutical composition comprises 6.5% w/v sucrose (65 mg/mL sucrose), 0.03% w/v polysorbate 80 (0.3 mg/mL polysorbate 80), 20 mM histidine (L-Histidine 1.06 mg/mL and L-Histidine monohydrochloride 2.78 mg/mL), a pH of 5.8, and a volume of 2-50 mL; and
(b) the anti-TTR antibody or an antigen-binding fragment thereof comprises a VH region having the amino acid sequence of SEQ ID NO: 11 and VL region having the amino acid sequence of SEQ ID NO: 12.

64. The pharmaceutical composition of any one of claims 1 to 60, wherein:
(a) the pharmaceutical composition comprises 8% w/v sucrose (80 mg/mL sucrose), 0.03% w/v polysorbate 80 (0.3 mg/mL polysorbate 80), 20 mM histidine (L-Histidine 1.06 mg/mL and L-Histidine monohydrochloride 2.78 mg/mL), a pH of 5.8, and a volume of 2-50 mL; and
(b) the anti-TTR antibody or an antigen-binding fragment thereof comprises a VH region having the amino acid sequence of SEQ ID NO: 11 and VL region having the amino acid sequence of SEQ ID NO: 12.

65. A pharmaceutical composition comprising a human anti-transthyretin (TTR) antibody or antigen-binding fragment thereof at a concentration of about 50 mg/mL to about 150 mg/mL, a histidine buffer with a pH of about 5.8, 6.5% or 8.0% w/v of sucrose, and 0.03% w/v of polysorbate 80;
wherein the anti-TTR antibody or antigen-binding fragment thereof is capable of binding mutated, misfolded, misassembled and/or aggregated TTR species and/or fragments thereof and does not substantially recognize physiological TTR species.

66. A pharmaceutical composition formulated for intravenous injection comprising 50 mg/ml of a human anti-transthyretin (TTR) antibody comprising a heavy chain variable (VH) region having the amino acid sequence of SEQ ID NO: 7 or 11 and light chain variable (VL) region comprising the amino acid sequence of SEQ ID NO: 8 or 12, wherein the pharmaceutical composition comprises 6.5% weight per unit volume (w/v) sucrose (65 mg/mL sucrose), 0.03% w/v polysorbate 80 (0.3 mg/mL polysorbate 80), 20 mM histidine (L-Histidine 1.06 mg/mL and L-Histidine monohydrochloride 2.78 mg/mL), and a pH of 5.8.

67. The pharmaceutical composition of claim 66, wherein the VH comprises the amino acid sequence of SEQ ID NO: 7 and the VL comprises the amino acid sequence of SEQ ID NO: 8.

68. The pharmaceutical composition of claim 66, wherein the VH comprises the amino acid sequence of SEQ ID NO: 11 and the VL comprises the amino acid sequence of SEQ ID NO: 8.

69. The pharmaceutical composition of claim 66, wherein the VH comprises the amino acid sequence of SEQ ID NO: 11 and the VL comprises the amino acid sequence of SEQ ID NO: 12.

70. The pharmaceutical composition of any one of claims 66 to 69, wherein the pharmaceutical composition has a volume of 2 ml.

71. The pharmaceutical composition of any one of claims 66 to 69, wherein the pharmaceutical composition has a volume of 20 ml.

72. A pharmaceutical composition formulated for intravenous injection comprising 50 mg/ml of a human anti-TTR antibody comprising a VH region having the amino acid sequence of SEQ ID NO: 7 or 11 and a VL region comprising the amino acid sequence of SEQ ID NO: 8 or 12, wherein the pharmaceutical composition comprises 8% w/v sucrose (80 mg/mL sucrose), 0.03% w/v polysorbate 80 (0.3 mg/mL polysorbate 80), 20 mM histidine (L-Histidine 1.06 mg/mL and L-Histidine monohydrochloride 2.78 mg/mL), and a pH of 5.8.

73. The pharmaceutical composition of claim 72, wherein the VH comprises the amino acid sequence of SEQ ID NO: 7 and the VL comprises the amino acid sequence of SEQ ID NO: 8.

74. The pharmaceutical composition of claim 72, wherein the VH comprises the amino acid sequence of SEQ ID NO: 11 and the VL comprises the amino acid sequence of SEQ ID NO: 8.

75. The pharmaceutical composition of claim 72, wherein the VH comprises the amino acid sequence of SEQ ID NO: 8 and the VL comprises the amino acid sequence of SEQ ID NO: 12.

76. The pharmaceutical composition of any one of claims 72 to 75, wherein the pharmaceutical composition has a volume of 2 ml.

77. The pharmaceutical composition of any one of claims 72 to 65, wherein the pharmaceutical composition has a volume of 20 ml.

78. A pharmaceutical composition formulated for intravenous injection comprising about 50 mg/ml of a human anti-transthyretin (TTR) antibody comprising two heavy chains and two light chains, wherein each heavy chain has the amino acid sequence of SEQ ID NO: 9, and each light chain has the amino acid sequence of SEQ ID NO: 10, wherein the pharmaceutical composition comprises 6.5% weight per unit volume (w/v) sucrose (65 mg/mL sucrose), 0.03% w/v polysorbate 80 (0.3 mg/mL polysorbate 80), 20 mM histidine (L-Histidine 1.06 mg/mL and L-Histidine monohydrochloride 2.78 mg/mL), and a pH of 5.8.

79. The pharmaceutical composition of claim 78, wherein the heavy chain of the antibody lacks the C-terminal lysine, wherein the glutamine at the N-terminus of the heavy chain is modified as pyro-glutamic acid, and wherein the heavy chain is N-glycosylated.

80. The pharmaceutical composition of claim 79, wherein the heavy chain has the amino acid sequence of SEQ ID NO: 15.

81. The pharmaceutical composition of any one of claims 78 to 80, wherein the pharmaceutical composition has a volume of 2 ml.

82. The pharmaceutical composition of any one of claims 78 to 80, wherein the pharmaceutical composition has a volume of 20 ml.

83. A pharmaceutical composition formulated for intravenous injection comprising about 50 mg/ml of a human anti-transthyretin (TTR) antibody comprising two heavy chains and two light chains, wherein each heavy chain has the amino acid sequence of SEQ ID NO: 9, and each light chain has the amino acid sequence of SEQ ID NO: 10, wherein the pharmaceutical composition comprises 8% weight per unit volume (w/v) sucrose (80 mg/mL sucrose), 0.03% w/v polysorbate 80 (0.3 mg/mL polysorbate 80), 20 mM histidine (L-Histidine 1.06 mg/mL and L-Histidine monohydrochloride 2.78 mg/mL), and a pH of 5.8.84.
The pharmaceutical composition of claim 83, wherein the heavy chain of the antibody lacks the C-terminal lysine, wherein the glutamine at the N-terminus of the heavy chain is modified as pyro-glutamic acid, and wherein the heavy chain is N-glycosylated.

85. The pharmaceutical composition of claim 84, wherein the heavy chain has the amino acid sequence of SEQ ID NO: 15.

86. The pharmaceutical composition of any one of claims 83 to 85, wherein the pharmaceutical composition has a volume of 2 ml.

87. The pharmaceutical composition of any one of claims 83 to 85, wherein the pharmaceutical composition has a volume of 20 ml.

88. A kit comprising the pharmaceutical composition of any one of claims 1 to 87 and instructions for use thereof.

89. The pharmaceutical composition of any one of claims 1 to 87 or the kit of claim 88 for use in treating or preventing transthyretin-mediated amyloidosis (ATTR) in a human subject.

90. A method of treating ATTR in a human subject comprising administering the pharmaceutical composition of any one of claims 1 to 87 to the human subject.

91. A method of treating ATTR with cardiomyopathy (ATTR-CM) in a human subject comprising administering the pharmaceutical composition of any one of claims 1 to 87 to the human subject.

92. A method of treating transthyretin-mediated amyloidosis (ATTR) in a human subject comprising administering a pharmaceutical composition comprising 50 mg/ml of a human anti-TTR antibody comprising a VH region having the amino acid sequence of SEQ ID NO: 7 or 11 and a VL region comprising the amino acid sequence of SEQ ID NO: 8 or 12, wherein the pharmaceutical composition comprises 6.5% w/v sucrose, 0.03% w/v polysorbate 80, 20 mM histidine, and a pH of 5.8.

93. The method of claim 92, wherein the VH comprises the amino acid sequence of SEQ ID NO: 7 and the VL comprises the amino acid sequence of SEQ ID NO: 8.

94. The method of claim 92, wherein the VH comprises the amino acid sequence of SEQ ID NO: 11 and the VL comprises the amino acid sequence of SEQ ID NO: 8.

95. The method of claim 92, wherein the VH comprises the amino acid sequence of SEQ ID NO: 11 and the VL comprises the amino acid sequence of SEQ ID NO: 12.

96. The method of any one of claims 92 to 95, wherein the method comprises administering a dose of the pharmaceutical composition providing 30-60 mg/kg of the human anti-TTR antibody or up to 7500 mg of the human anti-TTR antibody to the subject.

97. A method of treating ATTR in a human subject comprising administering a pharmaceutical composition comprising 50 mg/ml of a human anti-TTR antibody comprising a VH region having the amino acid sequence of SEQ ID NO: 7 or 11 and a VL region comprising the amino acid sequence of SEQ ID NO: 8 or 12, wherein the pharmaceutical composition comprises 8% w/v sucrose, 0.03% w/v polysorbate 80, 20 mM histidine, and a pH of 5.8.

98. The method of claim 97, wherein the VH comprises the amino acid sequence of SEQ ID NO: 7 and the VL comprises the amino acid sequence of SEQ ID NO: 8.

99. The method of claim 97, wherein the VH comprises the amino acid sequence of SEQ ID NO: 11 and the VL comprises the amino acid sequence of SEQ ID NO: 8.

100. The method of claim 97, wherein the VH comprises the amino acid sequence of SEQ ID NO: 11 and the VL comprises the amino acid sequence of SEQ ID NO: 12.

101. The method of any one of claims 97 to 100, wherein the method comprises administering a dose of the pharmaceutical composition providing 30-60 mg/kg of the human anti-TTR antibody or up to 7500 mg of the human anti-TTR antibody to the subject.

102. A method of treating ATTR in a human subject comprising administering a pharmaceutical composition comprising 50 mg/ml of a human anti-TTR antibody comprising two heavy chains and two light chains, wherein each heavy chain has the amino acid sequence of SEQ ID NO: 9, and each light chain has the amino acid sequence of SEQ ID NO: 10, wherein the pharmaceutical composition comprises 6.5% weight per unit volume (w/v) sucrose, 0.03% w/v polysorbate 80, 20 mM histidine, and a pH of 5.8.

103. The method of claim 102, wherein the heavy chain of the antibody lacks the C-terminal lysine, wherein the glutamine at the N-terminus of the heavy chain is modified as pyro-glutamic acid, and wherein the heavy chain is N-glycosylated.

104. The method of claim 103, wherein the heavy chain has the amino acid sequence of SEQ ID NO: 15.

105. The method of any one of claims 102 to 104, wherein the method comprises administering a dose of the pharmaceutical composition providing 30-60 mg/kg of the human anti-TTR antibody or up to 7500 mg of the human anti-TTR antibody to the subject.

106. A method of treating ATTR in a human subject comprising administering a pharmaceutical composition comprising 50 mg/ml of a human anti-TTR antibody comprising two heavy chains and two light chains, wherein each heavy chain has the amino acid sequence of SEQ ID NO: 9, and each light chain has the amino acid sequence of SEQ ID NO: 10, wherein the pharmaceutical composition comprises 8% weight per unit volume (w/v) sucrose, 0.03% w/v polysorbate 80, 20 mM histidine, and a pH of 5.8.

107. The method of claim 106, wherein the heavy chain of the antibody lacks the C-terminal lysine, wherein the glutamine at the N-terminus of the heavy chain is modified as pyro-glutamic acid, and wherein the heavy chain is N-glycosylated.

108. The method of claim 107, wherein the heavy chain has the amino acid sequence of SEQ ID NO: 15.

109. The method of any one of claims 106 to 108, wherein the method comprises administering a dose of the pharmaceutical composition providing 30-60 mg/kg of the human anti-TTR antibody or up to 7500 mg of the human anti-TTR antibody to the subject.

110. A medicament comprising the pharmaceutical composition of any one of claims 1 to 87 for use in a method of treating ATTR in a subject in need thereof, preferably for use in a treatment of a subject having ATTR amyloidosis with cardiomyopathy (ATTR-CM).

111. A sterile pharmaceutical container comprising the pharmaceutical composition of any one of claims 1 to 87 or the medicament of claim 110, preferably wherein the container is a single-use glass vial, preferably sealed with a rubber stopper and an aluminium-plastic cover flip-off cap, or infusion bottle, or infusion bag, or dosing syringe in a dosing pump comprising about 100 mg of the antibody at about a 50±5 mg/mL to about 150 mg/mL concentration, such as a concentration of about 100 mg/mL, optionally wherein the vial contains an approximate 10% volume overfill.

112. The medicament of claim 110 or the container of claim 111, wherein the pharmaceutical composition is ready-to use for administration to a subject in need thereof, preferably via intravenous infusion.

113. The medicament or container of claim 112, wherein the pharmaceutical composition is diluted prior to infusion in a solution of glucose or a polymer thereof, preferably wherein the polymer is dextran.

114. The medicament or container of claim 113, wherein the pharmaceutical is diluted to a concentration of about 1 mg/mL to about 50 mg/mL.

115. The medicament or container of claim 113 or 114, wherein the concentration of the glucose or the polymer thereof is 5% w/v.

116. Use of a pharmaceutical composition in the manufacture of a medicament for treating or preventing ATTR in a subject, wherein the pharmaceutical composition comprises:
(a) a human anti-TTR antibody or antigen-binding fragment thereof capable of binding aggregated TTR species thereof and does not substantially recognize physiological TTR species, and
(b) one or more of sucrose, polysorbate 80, and a polar excipient.

117. The use of claim 116, wherein the human anti-TTR antibody or antigen-binding fragment thereof is present in the medicament at a concentration of about 50 mg/mL to about 150 mg/mL and wherein the medicament comprises a histidine buffer with a pH of about 5.8, 6.5% or 8.0% w/v sucrose, and 0.03% w/v polysorbate 80.

118. The use of claim 116 or 117, wherein the pharmaceutical composition is the pharmaceutical composition of any one of claims 1 to 87.

119. Use of a pharmaceutical composition in the manufacture of a medicament for treating ATTR in a human subject, wherein the pharmaceutical composition comprises about 50 mg/ml of a human anti-TTR antibody comprising a VH region having the amino acid sequence of SEQ ID NO: 7 or 11 and a VL region comprising the amino acid sequence of SEQ ID NO: 8 or 12; wherein the pharmaceutical composition comprises 6.5% w/v sucrose; 0.03% w/v polysorbate 80; and 20 mM histidine, and a pH of 5.8.

120. The use according to claim 119, wherein the VH comprises the amino acid sequence of SEQ ID NO: 7 and the VL comprises the amino acid sequence of SEQ ID NO: 8.

121. The use according to claim 119, wherein the VH comprises the amino acid sequence of SEQ ID NO: 11 and the VL comprises the amino acid sequence of SEQ ID NO: 8.

122. The use according to claim 100, wherein the VH comprises the amino acid sequence of SEQ ID NO: 11 and the VL comprises the amino acid sequence of SEQ ID NO: 12.

123. Use of a pharmaceutical composition in the manufacture of a medicament for treating ATTR in a human subject, wherein the pharmaceutical composition comprises about 50 mg/ml of a human anti-TTR antibody comprising a VH region having the amino acid sequence of SEQ ID NO: 7 or 11 and a VL region comprising the amino acid sequence of SEQ ID NO: 8 or 12; wherein the pharmaceutical composition comprises 8% w/v sucrose; 0.03% w/v polysorbate 80; and 20 mM histidine, and a pH of 5.8.

124. The use according to claim 123, wherein the VH comprises the amino acid sequence of SEQ ID NO: 7 and the VL comprises the amino acid sequence of SEQ ID NO: 8.

125. The use according to claim 123, wherein the VH comprises the amino acid sequence of SEQ ID NO: 11 and the VL comprises the amino acid sequence of SEQ ID NO: 8.

126. The use according to claim 123, wherein the VH comprises the amino acid sequence of SEQ ID NO: 11 and the VL comprises the amino acid sequence of SEQ ID NO: 12.

127. Use of a pharmaceutical composition in the manufacture of a medicament for treating ATTR in a human subject, wherein the pharmaceutical composition comprises 50 mg/ml of a human anti-TTR antibody comprising two heavy chains and two light chains, wherein each heavy chain has the amino acid sequence of SEQ ID NO: 9, and each light chain has the amino acid sequence of SEQ ID NO: 10; wherein the pharmaceutical composition comprises 6.5% w/v sucrose; 0.03% w/v polysorbate 80; and 20 mM histidine, and a pH of 5.8.

128. The use according to claim 127, wherein the heavy chain of the antibody lacks the C-terminal lysine, wherein the glutamine at the N-terminus of the heavy chain is modified as pyro-glutamic acid, and wherein the heavy chain is N-glycosylated.

129. The use according to claim 128, wherein the heavy chain has the amino acid sequence of SEQ ID NO: 15.

130. Use of a pharmaceutical composition in the manufacture of a medicament for treating ATTR in a human subject, wherein the pharmaceutical composition comprises 50 mg/ml of a human anti-TTR antibody comprising two heavy chains and two light chains, wherein each heavy chain has the amino acid sequence of SEQ ID NO: 9, and each light chain has the amino acid sequence of SEQ ID NO: 10; wherein the pharmaceutical composition comprises 8% w/v sucrose; 0.03% w/v polysorbate 80; and 20 mM histidine, and a pH of 5.8.

131. The use according to claim 108, wherein the heavy chain of the antibody lacks the C-terminal lysine, wherein the glutamine at the N-terminus of the heavy chain is modified as pyro-glutamic acid, and wherein the heavy chain is N-glycosylated.

132. The use according to claim 131, wherein the heavy chain has the amino acid sequence of SEQ ID NO: 15.

133. An article of manufacture comprising one or more container(s) of any one of claims 111 to 115, and a label or package insert, wherein the label or package insert prescribes that the antibody is indicated for the treatment of ATTR, especially wild-type or hereditary transthyretin-mediated amyloid cardiomyopathy (ATTR-CM).

134. The article of manufacture of claim 133, wherein the formulation of the antibody is provided as a concentrate for solution for infusion and preferably presented as a preservative-free, clear to opalescent and colorless to pale yellow solution, provided as 100 mg/2 mL in a vial.

135. A method of making a pharmaceutical composition of a human anti-transthyretin (TTR) antibody or antigen-binding fragment thereof, which is capable of binding aggregated TTR species and does not substantially recognize physiological TTR species, the method comprising formulating the antibody in a buffer comprising sucrose and/or polysorbate 80.

136. The method of claim 135, wherein the pharmaceutical composition is the pharmaceutical composition of any one of claims 1 to 87.


SEQUENCE LISTING (20 sequences)

SEQ ID NO: 1 - protein, 5 aa, synthetic construct
SRSSY

SEQ ID NO: 2 - protein, 16 aa, synthetic construct
GIYHSGNTYDNPSLKS

SEQ ID NO: 3 - protein, 10 aa, synthetic construct
IVPGGDAFDI

SEQ ID NO: 4 - protein, 11 aa, synthetic construct
RASQSVGTYLN

SEQ ID NO: 5 - protein, 7 aa, synthetic construct
AASSLQS

SEQ ID NO: 6 - protein, 9 aa, synthetic construct
QQSYSSPPT

SEQ ID NO: 7 - protein, 120 aa, synthetic construct
QLQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSS

SEQ ID NO: 8 - protein, 107 aa, synthetic construct
DIQMTQSPSSLSASVGDRVTIACRASQSVGTYLNWYQQKRGKAPKLLIFAASSLQSGVPSRFSGSGSGTDFTLTISSLQPEDFATYYCQQSYSSPPTFGQGTKVEIK

SEQ ID NO: 9 - protein, 450 aa, synthetic construct
QLQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTIACRASQSVGTYLNWYQQKRGKAPKLLIFAASSLQSGVPSRFSGSGSGTDFTLTISSLQPEDFATYYCQQSYSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 119 aa, synthetic construct
LQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSS

SEQ ID NO: 12 - protein, 106 aa, synthetic construct
DIQMTQSPSSLSASVGDRVTIACRASQSVGTYLNWYQQKRGKAPKLLIFAASSLQSGVPSRFSGSGSGTDFTLTISSLQPEDFATYYCQQSYSSPPTFGQGTKVEI

SEQ ID NO: 13 - protein, 449 aa, synthetic construct
QLQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 449 aa, synthetic construct
LQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 448 aa, synthetic construct
LQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 13 aa, synthetic construct
QLQLQESGPGLVK

SEQ ID NO: 17 - protein, 7 aa, synthetic construct
DTLMISR

SEQ ID NO: 18 - protein, 16 aa, synthetic construct
VVSVLTVLHQDWLNGK

SEQ ID NO: 19 - protein, 22 aa, synthetic construct
GFYPSDIAVEWESNGQPENNYK

SEQ ID NO: 20 - protein, 8 aa, synthetic construct
SLSLSPGK